SEQUENCE LISTING (55 sequences)

SEQ ID NO: 1 - protein, 358 aa, Soybean
MACLLRARNPCYSLMRRIRIRCMSNVPENTVYSGPTSQSSTKRVTLSQLRQKHRASQPITMVTAYDYPAAVHLDTAGVDICLVGDSAAMVVHGHDTTLPITLEEMLVHCRAVARGAKTPLLVGDLAFGTYESSSNQAVDTAVRMLKEGGMDAIKLEGGSPSRIVAAKAIVEAGIAVMGHVGLTPQAISVLGGFRPQGKNVASAVKVVETALALQEAGCFSVVLECVPAPVAAATTATLQIPTIGIGAGPFCSGQVLVYHDLLGMLQHPHHAKVTPKFCKQYARVGDVINKALLEYKEDVINGSFPDAHHSPYKISKTDADVFSTELQRLGLDKAASAASEAVQKMDTTKSTSEGKQIK

SEQ ID NO: 2 - protein, 335 aa, Soybean
MLLSPRNPLEASYQRLKVKVDDTEPTKYFICHSCSKGSDLLLSSFDGARCSCRKLMQKKMELLEESKDEASVVDGIFVKGDAMFLIFDDLTVLRSSPSDSVQRPLQLGHKNFSSKLEEKYSDVGTKEIFSILKEALTSKSPLSNVFLENRESNSSYSFSSHTGSDACGPFHWKEDDAPKYWCGTVADKDNEGRTMISKINDMLQYPAKKLKLFEPRCSDGAKEAGVGFMKRPCLFVVMDDLKVIPMTTTSSIEYLQKLEEENVELDDLVEIRKRLEALNLLRASLTSDKAAFTRSLFYLLKKWKCQRCIPFYGVYVCKKKRSRKKKVMEEKESER

SEQ ID NO: 3 - protein, 117 aa, Soybean
MAKDPLGHQGMMSFQKEEMKCGYKKRKKGIKEMRQLQGVERGLRGLLEVIAKEPHVKSHEPSCYVGDLKQIYTLDEDMCSWILPLQRIGMASTDTAPPKVDFDSHKAQMCANLISTK

SEQ ID NO: 4 - protein, 56 aa, Soybean
METAVSSALLRKLVDYIRYRRNIDEVDEKSIMAALEYSTVNIIGVHMDQVGWIREL

SEQ ID NO: 5 - protein, 1561 aa, Soybean
MESVLSPIVERTFNFVVDPIIRQLAYILRCSQNVDELLTSFKSLELEKESIDRRCDQAENNLQNIEAKVKVWSRKVDEFKTELEKFWNDEGHRKTGLSNVLFLFPYFWNRHRLGRQAKKMAEIVKNLIDESAKFNEVSYSDNLTSNDFTLSNPGYMGFASRHSTVEKIIAKLEDSSVRMIGLHGSGGMGKTTLIKAIAKKAMDKKLFNVVAVSEITANPNPQKIQEDIASALRLRLEGEGENARAHRLMTRLKQEKENTLLILDDLWDRLDLNKLGIPLDGDVDDNDLNTKTSNAKLGPKEATKEKSLGDYKGCKILLTSRDKNVLTDKMEVKSTFCVEELDDDDALRLFRKEARIQGEMSQWKQEIVKKYCAGLPMAIVTVGRALRDKSDSEWEKLKNQDLVGIQNSMEISVKMSYDRLENEELKSIFFLCAQMGHQPLIMDLVKYCFGLGILEGVYSLGEARSKISTSIQKLKDSGLVLYESPSINFNMHDLVRDAALSIARKEQNVFTLRNGKLDDWPELERCTSISICNSDIIDELPEEINCPQLKFFQIDSHDSSLKIPDSFFKGMKKLKVLMLTGIQLSSLPSSIESLSDLRLLCLERCTLDHKLSIIGKLKKLRILSFSGSRIENLPAELKDLDKLQLLDISNCSVVKRIPPKLMSRLTSLEELYVRKSFIEVSVEGDRNQCQISFLSELKHLHQLHVVDLSIPCAQVFPKELFFDKLNDYKIEIGNFKTLSAGDFRMPNKYEKFKSLALELKDDTDNIHSQKGIKLLFKRVENLLLGELNGVQDVINELNLDGFPHLKHFSIINNASIKYIINSKDLFYPQDVFPKLESLCLYELRKIEMIYFSSGTEMICFSPFTDCSFTKLKTIKVEKCEQLKNLFSFCMVKLLASLETIGVSNCGSLEEIIKIPDNSDKIEFLKLMSLSLESLSSFTSFYTTVEGYSTNRDQIQITVMTPPLFGELVEIPNLENLNLISMNKIQKIWSDQPPSNFCFQNLIKLVVKDCENLRYLCSLSVASSLRKLKGLFVSNCKMMEKIFSTEGNSADKVCVFPRLEEIHLDDMDMLTDIWHVEVSADSFSSLTSVNIKSCNKLDKIFPSHMEGWFASLNSLKVSYCMSVEVIFEIKDSQQADASGGIDTNLQVVHVSCLPKLEQVWSRDPGGILNFKKLQSIEMFRCGRLRNVFPASVAKDVPKLEYMSVIWCDGIVEIVACEDGSETNTEQLVFPELTYMCLGCLSSIQHFYRGRHPIECPKLKKLEVGKCNEKLKTFGTGERSNEEDEAVMSAEKIFPNLECLYIDFDEAQKWLLSNTVKHPMHRLKELNLYYVNDDERLCQILCRMPNLEKLYLREDEHLLKESSESRLGTVLQLKELVLWWSKIKDIGFEREPVLQRLELLSLHWCHKLRNLAPPSVSLAYMTNLKVGYCKGLRNLMASSTAKSLVQLKSMKISHCNKLEEIVSDEGNEEAEQIVFGKLITIELEYLPKLKSFCSYKKCEFKFPSLEGLIVRECPMMHTFTEGGARAAKLQNIVTANEEGKEEAKWQWEGDLNATIQKGFNKSL

SEQ ID NO: 6 - protein, 469 aa, Soybean
MEMKFLIFQTKFLESASTASSLSLIDSPLQVIWLDSRRIPKSCFSNLNSLTVDGCQFLTDVVIPFYLLPFLTNLQELQVRNCRSVKSIFDVKTTTGLGAAAFPRPLPFSLKKLTLERLPKLENVWNEDPHGILTMQLLQHVIVEKCKCLTSVFPASLAKDLEKLVVKDCEGLIEIVAEDNADPREANLELTFPGPCVRSLKLQGLPKFKYFYYCSLQCDMFQTPTKDEMPTSNLQCLSLGEKGLEMIKRGEFQRNFIHKLQVLTLCFHIGSNVFPYEILQLAPNIKKLVVCDGSFKEIFCFDSLNVDEAGLLLQLKVLCLDSLPELVSIGLENSSIQPLMGNLETLEVIGCSSLKDLVPSTVSFSNLTYLKVERCNCLLYLLTTSTAKSLTRLKRMEIEWCDSIEEVVSKEGGESHEDEIIFPQLNCLKLKCLPKLRSFYKGSLLSFPSLEELSVIYCEWMGNIMSRCP

SEQ ID NO: 7 - protein, 569 aa, Soybean
MLWKSADRRFNIDLKDSRLQEIWLRLHSLHIPPHFCFPKLHTLIVDGCHFLSDAVLPFSLLPLLPELKTLEVRNCDFVKIIFDVTTMGPLPFALKNLILERLPNLENVWNSNVELTFPQVKSLALCDLPKLKYDILKPFTHQEPHALNQVCIQKLTPNIEHLTLGQHELNMILSGEFQGNHLNKLKVLALFFHIESDVFVQRVPNIEKLEVLGGFFREIFCFDSLNVDEAGLLSQLKVICSDSLPELVSIGSENSGIVPFLRNLETLQVISCLSSINLVPCTVSFSNLTYLKVKSCKSLLYLFTSSTARTLGQLKTMEISWCDSIEEIVSSTEEGDESDENEIIFQQLNCLVLKELGKLRRFYKGSLSFPSLEEFTVRDCERMESLCAGNDSNIADLYFIALISKICWVLFFLIESNVFLQRVPNLEKLVILRGSFKETFHFPELKVLPLESLGELASNGSENSWNVSFSNLTYLEVDSCKNMLYLFTSSTAISLTQLKTMKKLLQREFKFPILGRIVGNPLQQDGNCVQRFSDAIPLENDLNSTMRKEFQPTAKTASLPKRKWKPKRV

SEQ ID NO: 8 - protein, 124 aa, Soybean
MKGVLVKHRKLKRQGKGPMITMNSVKITKLQLVNTTMTPQPAANMSLVASFRYGNTTTSLYYHGVLVGEPRGPPGRAKARRTLRMNVTIDVITDRVVSWAGEDLELDISTQAIKEQSCKRKVKL

SEQ ID NO: 9 - protein, 69 aa, Soybean
MQQLQNEGSIHKGEYREANIKKKRNGKIEYFGEEQEISSFLVCLTTKILMMELVLQVKGGMALWLWWPM

SEQ ID NO: 10 - protein, 2467 aa, Soybean
MDAVSSALLEPVTNSLLDLLKKQLDYIHYSRNFDELRECVKQLKLVKEKVDHQCEEAFKNGHEIEGKAREWLGKVGKFETEVEKYWNDDGHKKTRFSNYLFLYFCHRLGRLAKKMAVEGKKITDDCPKSDEIAHRVYVTSNDAILSNNDLMDFGSRKSIMEQIMATLVEDPTVKMIGVYGRSGVGKSTLIKAIAEIARDKKLFNVVAFSEITDNPNLKQVQEDIAYPLGLTLEGESENVRADHLRRRLKKEKENTLIILDDLWDRLDLNRLGIPLDGDVDDNDLSKKTNSDKQGPKGPTKEKSLGDYKGCKILLTSRKQNVLTDKMEVKLTFCVEELDEKDALKLFRKEAGIHGEMSKSKQEIVKKYCSGLPMAIITVGRALRDKSDSEWEKLKNQDLVGDQNPMEISVKMSYDHLENEELKSIFFLCAQMGHQPLIMDLVKYCFGLGILEGVYSLGEARGKISTSIQKLKNSGLVLDGSSSIHFNMHDLVRDAALSIAQKEHNAFTLRNGKLDDWPELERCTSIFICNSVIIDELPEEINCPQLKFFQIDSDDSSLKIPNSFFKGMKKLKVLMLTGIQLSSLPSSIESLSDLRLLCLERCTLDDNLSIIGKLKKLRILSFSGSRIENLPAELKNLDKLQLLDISNCSVVKRIPPQLMSRLTSLEELYVRKCFMEVSEEGERNQCQISFISELKHLHQLQVVDLSIPCAEVFPKELFFDNLSDYKIEIGNFEMLSAGDFRMPNKYENFKSLALELKDDTDNIHSQKGIKLLFKRVENLLLGELNGVQDVINELNLDGFPQLKHLSIVNNPSIKYIINSKDLFYPQDVFPKLESLCLHELNKIEMIYFSSGTEMICFSPFTDCSFTKLKTIKVEKCDQLKNLFSFCMVKLLASLETIGVSNCGSLEEIIKIPDNSDKIEFLKLKSLSLQSLSSFTSFYTIEGSSTDRDEIQITVAENEHSEMAPPLFGELVEIPNLENLNLISMNKIQKIWSDQPPSNFCFQNLIKLDVNGCHNLRYLCSLSVASSLRKLKGLFVRECQMMEKIFSTEGNSADRVCVFPKLEEIHLKEMDNLTDIWQAELSADSFSSLTSVNIKSCNKLDKIFPSHMEGWFASLNSLKVYSCRSVKVIFEIKDSQQADASGGIDTNLQVVDVRVLPKLEQVWSRDPGGILNFKKLQSIEMFGCGRLRNVFPASVAKDVPKLEYMSVSECHGIVEIVACEDGSETNTEQLVFPELTYMNLSGLSSMQHFYRGRHPIECPKLKKMKIYNCSKKLKTFGTGERSNEEDEAVMSAEKIFPNLECLYIDFDEAQKWLLSNTVKHPMHRLKELHLRKVNDGERLCQILYRMPNLEKLYLWYAEHLLKESSESRLGTVLQLKELVLSQSKIKDIGFEREPVLQRLELLSLNGCDKLRNLGPPSVSLAYLTNLEVWYCKGLRNLMASSTAKSLVQLKSMKIRGCDELEEIVSNEGNEEAEQIVFGKLITIELEGLKKLKSFCSYKKCEFKFPSLEVLIVRECRMMERFTEGGARAPKLENIVSAKEEGKEEAKRQWEGDLNATIQNKILESARTDKRLTVSPLLQEIWLGSRPIPKSCFSNLKSLTVDGCQLLTDVVIPFYLLPLLTNLQELHVRKCGSVKSIFDVKTAMGLGAAAFPRPLPFSLKKLTLERLPKLENVWNEDPHGILTMQLLQHVIVEKCKCLTSVFPASVAKDLEILVVKDCEELMEIVAEDNADPREDNLELTFPCPCVRSLKLQGLPKFKYFYYCSLQCDMFQTPTEDEMPTSNLKCLSLGEKGLEMIKRGEFQRNFIHKLQVLTLCFHNGSDVFPYEILQLAPNIEKLVVYNASFKEINVDYTGLLLQLKDLCLESLPELVSIGLENSSIQPLLGNLETLEVIGCSSLKDLVPSTVSFSNLTYLEVERCHCLLYLFTSSTARSLGQLKRMEIKWCDSIEEVVVSKERDESHENEIIFPQLKCLKLEGLRKLRRFYRGSLLSFPSLEELSVIDCRWMETLCPGTLKADKLVQVQLEKSYPRSSHSDAIKLENDLNSTMREAFRKKLWKSADWESVLDLKDSPLQEIWLRLHSLHIPPHFCFTYLHTLIVDGCHFLSDAVLPFSLLPLLPNLETLAARNCDRVKIIFDVTTMGPLPFALKTLYLERLPNLENVWNSNVELTFPQVESLALCDLPKLKYDMLKPFTHLNQLCIQKLTPNIEHLTLGQHELNMILSGEFQGNHLNKLKVLALFFHIETDVFLQRVPNIEKLEVCDGSFKEIFCFDSHNVDEDGLVSQLKVICSDSLPELVSIGSENSGIVPFLRNLETLQVISCLSSINLVPCTVSFSNLTYLQVQNCKSLLYLFTSSTARSLGQLKTMEIGWCDSIEEIVSSTEEGDESDENEIIFQQLNCLVLQELGKLRRFYKGSLSFPSLEEFTVIHCERMESLCAGNDSNIRVRGDGFNKKKMVRGDQQIKWPVLNDPGE

SEQ ID NO: 11 - protein, 314 aa, Soybean
MVWVFLIGFVDINWIFMTREVEDAHARINENALGWSRTLALQVLQSMNISFDLPILKGDNYKVSKERILLHLGWMDIVYAIRKDELPAITETSEPDVVDLYEKWEKYNRLSVMFIKTNIFASIRGSVDQHDKVKDLLKAIDEQFMTSEKSLTSTLIMQFSSIKLTGTGGVREHIMRLRDIVAQLKTLEVTMSESFLVHFILCTLPQQYTHFKISYNTHKDKWSINELMTMCVQEDERLIMEEGEKVNLTTSTFGKDRKKFVGTNKGRIPTQPTIKKESKCFFCKKKGHIKKDCPKFKSGFEKKGYGKPKKANGK

SEQ ID NO: 12 - protein, 603 aa, Soybean
MRSNKGREYYGRYTEDGQPPGPFAKFLQEHEIVSQYTMRGSPNQNGVAERRNQTLLDMVRSMRSNVKLPQFLWIDALKITAYILNRVPTKVVSKTPFELFKEKKLDPRTITGYFIGYSEKSNGYRFYCQSHNTRIVESRNAKFLENDLISGSDQFQNISSERDHYEAEPSGTSNRLVVIPTPQVKMGVRQPVIEVPQVVESDHVDQVVYEEQHDDIEQTGEELVEQVSQQDNQAALRRSTRVKKTEIPSDYVVYLQESDYNIGAKNDPETFSQAMSSKKSNLWCNVMRDEMDSMASNQFWDLVELPVGVKPIGYVKMTFQNGDLEEEVYMKQPEGFLSSVGEHLVCKLNKSIYGLKQASRQWYLKFHEVISSFSFEENVMDHCIYQKLPSVAPIEKGDKFDLGQSPKNDFEREHMKNIPYASAIGSLMFALDLILHSLLESWKDIKVPGIDHWKAAKKVMRYLQGTKDYMLIWWSCIMESSKQSLIATSTMKAEFVSCFEATSHGVWLKSFISGLRVVDSISRPLKLYCDNSVAVFIAKNNKSGSRSKHIDIKYLAVRKRVKERKVVIEHISTDMMIVDPLTKGMPPKNFKDHVVRMGLDSIM

SEQ ID NO: 13 - protein, 393 aa, Soybean
MAGASVDRLDPFMPTSNLQCLSLGEKGLEMIKRGEFHERNFLHKLQVLTLGFNIGSTVFPYEILQLAPNIEKLVVCDGSFKEIFCFDSLNVDEAGLLLQLKVLCLDSLPEFVSIGLENSWIQPLLGNLETLEVIDCYSLKYLVPCTVSFSNLTYLQVQDCNSLLYLFTSSTARSLGQLKRMEIKRCYSIEEIVSKEGDESHENEIIFPQLNCLKLEGLRKLRRFYRGSLLSFPSLEELSVIDCKWMETLCPGTLKADKLVQVQLEESSDAIKLENDLNSTMREAFGKKLKVICSDSLPELVSIGSENSGIVPFLRNLETLQIVSSTEEGDESDENEIIFQQLNCLELEGLGKLRRFYKGSLSFPSLEKLTVNLCERMESLCAGNDSNIGVRGD

SEQ ID NO: 14 - protein, 213 aa, Soybean
MTTTQWVHELKNGKQKENSNSNSPPRPGIGVLRERHDLLQRRGQRRRHGGVPANGAGPVKLEPWVETTEMKVVSTLWHHPQHLRILVLAQTDRACAVVITFLNEWKLGVGVYDGLVEAGGDVVLAGVVVVVILVLGYEDHAREHDAIGGVAAVVVVVAAAEGAAAKVGGEDEGGEEEENAEGDGDGVAEAEVGEVRGGGGSGWRRREGVEGGH

SEQ ID NO: 15 - protein, 355 aa, Soybean
MVCQSLNPEVPNGNQNEVLMKTVEMVIQKIEEELDKLRETKQDPSLTPSVFQQGAFLSDILGLLESAAASHQQNEGNNQQNGGKQNFMGENASFTSIGHTCFAMKEELEEYMDYDVGEICNDDWKLAQKLMVHGCDPLPRRRCFSRSPKLYKQPFPVNESLWKLPDDRNVRWSQYQCKNFACLAGNATRKGFFKGADCFNLTNHEMPRWIFLIGLDLSVGTGTFAARMREFNVTIVSANINFGAPFNEMIALRGLVPLYLTINQRLPFFDNTLDLIHTTRFLDGWIDLVLLEFILYDWDRVLRPGGLLWIDSFFCLKAFKMLRYKKHKWVVVPKLDKDEQEMFFSVLEKPHRPFR

SEQ ID NO: 16 - protein, 364 aa, Soybean
MCPEPELRDPNLVMRNTINWDTERIARYLYLPSAPAESQEKAFDPCNCVRLFRVFSETMADESEHTAAPAAGESLLDKIAEKIHGHDDSSSSSDSDSDKKESSSIKEKVFRLFGREKPVHSVLGGGKPADVLLWRNKKISAGVLGVATAIWIFFELLEYHLLTLVCHISILLLAVLFLWSNAHTFIHKAPPRIPVVHIPEEPILQFASALTIEINRGFAALHAIGSGRDLKTFLIVIVGTWIISIVGSWCNFLTLFYIAFVLLHTVPVLYDKYEDKIDPLAEKALIEFKKQYAVFDEKIFYESNKNKKVKGSGEEDEKENELENPKESVEEEDGGTVRLVEGRLVEDEGRKREFCDLQKVYVTK

SEQ ID NO: 17 - DNA, 94411 bp, Soybean
  misc_feature (25299)..(25299): n is a, c, g, or t
cattttgaaagtacatttatccttaaaaaaaaaaaaatctaagcaataacataacagaaacattctttgttttcttttcctagtgggtgggagcagccgaacacacacaaaagctatgatcacaaatttggtaagtagttgttgaattcaaatcgagcttcgctttctcttccctgcactttgcactttgcactttgcactctgcacagcgcaatggcgtgtttgttgagagcgagaaacccctgttactctctgatgaggcgcattcgaatccggtgcatgagcaacgttcccgagaacacggtgtattcggggcccacctcgcagagctcgaccaagcgcgtcacgctgagccagttgcgccagaagcaccgcgcctcccagcccatcaccatggtcaccgcctacgactaccccgccgcggtgcacctcgacaccgccggcgtcgacatctgtctcgtcggcgactctgcggcgatggtggtccacggccatgacaccactctccccatcacgctcgaggagatgctcgtccactgccgcgccgtggcgcgtggagccaagacgccgcttctcgttggggacttggcttttgggacctacgagtccagctccaatcaggtcatgctcgtgcgtattgtttcactcattgttgaatggtttttaatgcttgtttgaataaacttatctggaagcactcgtagaaaagaaaaaaatgaaataaaagctattagcttagcatatgtattaattaaaaaaaagtttttttttcatcaccaaatgagtggatcaagtgacagagttgttctagcttaatctgtcttggaggaagagtttttgtcgcgtaagtgatcctaactggttcgaataggatgattgcctttattgtaagatgtggtatatacaaaaaaaacattttttagagaaaataatatgataaaacttctataaattaacttatgcgtgccttaattttagcttatagagaagattttgtttttttcttctcttaaagtgcttattgagaagtttattcaaatagatcattaagtgttgttgttttgattttaagcaggcggtggatactgcagttcgtatgttgaaagaagggggaatggatgccataaaactggagggagggtcgccttcgagaattgttgcagcaaaggctattgttgaggctggaattgctgtcatgggccatgtaggccttactccccaggccattagtgttttaggggggtttagacctcaaggaaagaatgttgctagtgctgtcaaggtctgccttcatgtgtctatttggattttgaattattttattgttgtcctgaaactgaaatgaacaagtttgtgtaggttgtagagacagcgttggctttacaagaagcagggtgcttttccgttgttctagagtgtgtgcctgcaccggttgcggctgcaaccacggcaacccttcaaattcctacaattggaattggggctggacccttttgcagtggacaagttagttggcctaattaccattttggaaaatgttctgttcttgacatgtacttttcgtaagagatgaatgcagcctacattagtgacagtaattttgtttaagctaaaatggcatggtcattggttcatttgaaggcgtttattgtttcatgaagttgctgttttggactttctgtttcatgtttaattgttgctcttaaatgatattgtaactttcatatgtatgtatgtagatactttttagtttttagttttgagaatatgtttagattggtcttgagtctcttcactgtctattacaagtgatggaattctaatttgattgtccctcagtcattttcagataattgaacaaagcattgtttgaaatttaattttacattacacttgttcttttggggaagagatgaaagaagccttttctgggtaaagggaaagcagaggatttctaattttgtttcattgttcatgcaggtgctagtttaccatgatctgcttggtatgctacaacaccctcaccatgcaaaggtatttgtagcatatcctaaccttgttttctctttcagacattatggaattcattgctaattttgagatgatgtatctttttcaggttactccaaaattttgtaagcagtatgcacgtgtaggagatgtcatcaataaagccttactggagtataaggaagacgtgataaatggttcatttcctgatgctcaccatagtccatacaaaatcagcaaaacagatgcagacgttttctcaactgagttgcaaaggttaggtctagacaaggcagcatctgcagcatctgaagcagttcagaagatggatacaaccaaatcaactagtgaagggaaacaaataaaataataatatagtatagctagagattcacccttttttcgctcgtaggtcccaatgtgataatttagcctgctacaaaatgaagttacataattaaatagaagttgagcagttgactaatggatagatagacaccattttcgtagggaaggccacttcaatttcaattgtttttcctcttatccttttgatcatcaatgccaatgcaaaaaatttcttaatgttacagaaacagcgtctttgatgtccaacattatgcatggaattttcaaaacaaccctgttttgaagcattgtaaaggagcaaaatgtgcgaaactagttgattttattgttattattatttatgcaattcattactgatgtaagccatcctttgagcttgtgtcagaagcaacttgcttccattgtaggtttagttttctttgcatattggtgctcaacccacttaatagcatgcttattttgttattcatattgatttattttatactattgggaaaagtcttgcatcactttactgtttaccaactttgtatgagatgagacatatagccattgatttgaatttaaacgagtaacaaaagccaaggaacttatatttaatgcattccagagctgttgaggtcgactgtgataaaatggttcttgtaaagcagactaaacttgaatgagcagttaccttagttatcattttaacttatcaaagtcaagaatgcattggctattgggaaatcagaaatgtttctgacatataccctgtactgtatctgtccacctgataatcacgagttccttttttttttcttccaaagagagggaaaatcattatgtgaaatgaagatgaaaacagttttacaatatatttaaacaaacgggaattgttcccaaaacagtcttccaagatccaacaataccaaatatgatggttttgagcttctccttaataaatcatggactctatgaagacctaatttacttaagtatgtttgttgtgtttgattcaatgtacgaataaccacatgttggaatgtggggatatctgcagtatttttgaagtcacttgttattaaatgttacgttgcaaagatcaacctggacaaaagttagataacatgagaagaaaacattataaattgttgaaatattttggataatccaattcttcaaatcattgaaaaaagatatggtgcttcaaattactatattttgatgaagctagtcagtatggttattgatatagaaattacagatataaaacttatgggttttttttttaatgaaatttagtagtgaaacttaaatcaaaggaatgaacaaattactagaaattagacacacaaaaaaattcatagaaatgaataatttaccagaagtaacataaacatctagattctattggaattgaatatatttaatttattgctataataattttaagaatttttataatttactaatttattatttttaaaaattacccataattttttaaaaatgatttactaactaaaatttttgcaatgtgaaatttattttatcttatatatttgaatattcaataacaaattcatacagatgagtaaaacaatcagctattgcatgggatgggtcatcactagtttaaaaataaatacaagttcatggactaaattaaaataaaaataagagatctaattaaaaatttgataaaaaaattcaaagacttgaaaataattaaacgttattattatctctcactttctttctcttccattacttttttctttctgcttctctttttcttgcatacatatacaccataaaagggaatgcacctttggcatttccacttcttcaataggtagaacaagctgcgtgtgaaagcagctttgtcggatgtcaaagaagccctcaacaggtttagtgcctaaccagaatagacataatgaacaacatcacttcaaaattgaaaaggctctagatgggtaacatggaagaaatattgtacaaagttaattatagccaatcgtgtttacttatttttttttttcttctaatttatttttctaggttcaatttatttgtcttttgtcttcctatttaaaggttcaattagttttttttaaaattaattagatcttttatttttttaaagttcaattaagtctctaatttctttaaatgaatttaattttagtttcatttaaaattatcaattttggtcctatcttttaaaaaaattggaaaaaattaaaaaaaatgagactatattaaatccattttaaaaataaaagatttaattcaacattttaaaagtaaaagatttaattaaactattaaaataaagacctaattaaaccttgaaataatgaaaaattaaactaaacctaaaaaatgtattaaaagataaaaaagtaatcaaacctaactaattatttgactaaattaagacagatgttaactaaaatcatcatatgaactgaaatttcttaaaccaatattgtggcttagaagaacaagtcgtttagagtggattatgaatattacctcgagtctcttcctgatttccaccaaatcatccaactcgacattctcctcctccagcttttgcagatactcaatgctagaagtagttgtcattggtatcactttcagatcatccatcacaacaaatagacacggcctcttcataaatcccacaccagcttcttttgctccatcagaacaccttggctcaaaaagtttcagtttttttgctggatattgtagcatatcatttatctttgaaatcattgtacgtccctcattatctttatctgctacagtgccacaccaatacttaggagcgtcgtcttcttgtaaaacatttagtggattgatcttacaaccaaatttaggagcgacccttggattaatcagaaaggatttgtttcatgacccgatgaaccatgatgggttgagattcttcacacttgtgcacaagttatcaatgcttcccaacgataacttgccattcataaggtttagaatagatccaagaggttctctgtgaagaagctaactagaaaatccacaaatctccctctgctccagcaaacagaatctcgttctttgatttgctaaacattatcttgatttctagagaacctttccaatgaaatgggccacaagcatctgagccagtatgtgatgagaaagagtatgatgaattagattctctattttctaagaatacattactaagaggagatttggaggttaatgcttcctttagtatgctaaatatctgaaaaaagaaaacaaaaaaaaaggcaagttttaagccagcgaagttacaattccctaaacaaggaattttacaccagtatttaaagtattacagtaaaggaaaggttctgttcatcaacaactttacaagctaactaaggaacttgaagaaggttatcagctacttacataagtggtaagcgcaagtcacaaaaatgttttgaattcaatgtatctggacttcctaccagaaaataatcattttttacctcatgaagtatatttttcattggcagaacttactagttctatttgtttcccaatacatattcatgctcaacctttttaagtgcacgctgagtctaactctaacatagtacataaaaatctcttatttgatttagaattcagaggattgaagagaaaaaaaaaggcaagatctttgtgcctaacctttgcgggccaaacaatattgggaggaatttattctttttgccactcctttcccctcctttaaaccaaacaactcatgtaagtaccacctgaaatgaatataaagcaatattacctccttcgtgccaacgtcactatacttttcttccagcttgctgctgaagtttttgtgtccaagttggaggggccgttgtacagagtcacttggggagcttcggagcactgtcaagtcatcaaagatcaagaacatggcatcccctttaacgaaaataccgtcgaccacagaagcctcgtccttggactcttccagcagctccattttcttttgcatcaattttctacaggaacaccttgccccatcaaaactactcagcaacaaatcacttccttttgaacagctgtgacaaatgaaatactttgtgggctctgtgtcatccaccttcacttttagtctctggtaagaggcttctaacgggttgcgtggagaaagcaacatttccagaaaacatcaggactcaaattttgcacactctcgtacagattgttgatgctaccaatctgtaccggttggttaaattgattcccaagccggatgatagttcccaatggaaggggtcaggaaactaaagagaacatcaacaaagtctccacttgcttctgccataactacacgcttttgttcattgtccaccctatatctcaaagggattgttgcttcttggcttgaagctgaaacatcaaattaaacattagtatattgtaaaggatcacaagatctacacaaggaacatgacctgatcacgcactcaacgcaacaagtttgtgtccctctcttgatgatacgaattttacttcaacatatctactttattttcctcacacattgttacttgggcgttcatagaagtcctttattttgcatatctatcctcccgctagaggctctcatacatgtgaaatttgagataagactcaatcaagtttatcctgacatgtcacacgtgattttttaagggataagcaaacattatcaagagtatcaagagtgttgcgtatacgtgtttggaactctaaacaaaaactaaacaacaacaaaattaattcggattttagtaaagttttttttttaaaaaaaaatagtttgaactttttgcaaattacttctaaattgatcgagttactaaacaatttagaatttggttttgaatgccaaaatcatcagtttcaaaaccaattttttcctaaatttaaacattttacagattttttaattagaaaatatcattgccttaatttaacaatactcaaaaattaaggttaagcaaccctattaaagagacttaaatatgttttattctagtactttaatatttttaattttaatttttaaatttttttatcatttctagtcttataagtttgtactttttaattataattactttaagattttaattttttatttgtaattttcataagttcacgtctagatagacaaaaaaaaaaatatatataattttataagaattaaaaataataaaatatcttataaggattaaaattaaaaaaaaacaatttccaaggactaaaattagaaaaagaggaaccaaaaataaaaaaacctaacttacaagggaaaaaaaatttttagcctattgttagaagtagaacttaaccaaattggccggatcgaactggcaataccagtcaattccgtatcataaaaatcaatcttgcaaagaaccggttaaatcagacaaagtaagcaagagctaattctttagcaaaattttcttaattagaaaatattaattttatgatgatcaatagtatgtcaattatatttatttagtttctaagtgtagttgctaatgaatattatcacgtgttaacaaaaatgttaaaatatatttgatttagtttcatatttgactttgaatattgcaaatgataagctattagttaaaacgttgatgattgagtaataaatttttaatcactactaactacaagaaatatatcttatgtcactttctacatcgattgttaaaaaattgaagtagaaagtagtgtgatgacagttttgcaattgaattcaactttataccaccaatttatctaaaatctatgttggcagcatattttaacatccattcaaaataaaattgatgttaaaaaggtattttaacgttgattataataagaattgatcttaaattgttttctatacttactaatttgagaagtccattttgctcttgcctggtctctactcctcaaacccttaaaatctcttttcttccttgaccttattaccaccacaaccttaaaccaccatcatgacctcgtgccaaaccccaaccacatggcaagagctacaccatccccattctcttctccttcaaaatacctagaacaccaagacaggacctcaccaccgccaccttcaaaccgccactgtcatgccaccattacttcatgtcttctcatccccatacatctttcattctcttttcccttttgtgccaccaagtgttgtattctaattctcgaataaagcacaacaaaactcttcacttcgttgaaatcagatttgcgcacatctgagctttgtgggagtcaaaatcaaccttgggtggagctgtgtctgtgcttgccatcccaatccgctaacaatcaccatacaccaatcatatcagtattcattaattttgacaacataagaaatcatcatataaaaaatttatcattcacatgttcatcgcacttatgaacatatttatagttcaacgaacataacaaaacattctaatatcatgaattcttataatcacctacccaaaatcaatgtgaatttcataaattaattttaattataaagcaacatcattctattcattatactatttatctaattgatattgtggtccttatctagtttcttaagttcttatatggtacctccttcatttcctattacacacctttatgacctttgtatgttgaaaatatgatatctagagttataccagctcaattaagttgattcttatctcattataaagttacgaatgttttatacaatttttatatttactccaagagctagttcgtaagtatattatgcctaaaattatgaacaaggttgaacttttaccaaattttgacaacttgacatttgccattaatttgatcacatctggagttgtagacaatattgttgagtgagacaaaagggattagttagataatatctaaagttatgactttcatgaaaaacacttttctaaattcagactctacaactgaattttagccattgaaatcgattaaaacagaggtcagatcaaggaagcagcccctgggtttgaattaacaaactacaaatctatatgtaatcgataccataaacccaggatcaacaaaacaactactgtgactagaattaattggttacaaaaccttgtaatcggttactttagtctctggaatgataaatttgtcccaaatacatctgttttattttttatttcgagaagagatgtagtcactgtacaagaaaatcaagctgaaagaatggagtttgtgttgtccacaatagtagaacaaacaattaattttacagtggacccgataatgtcttcgtgagtgttgtaaggactttagactaactcactattttaaaggtcattttcatggtttttcttttcttttttggatactctggctcacaactattacctcaccacatcaacattattgcatccttggattataccttaagctttactagacacttaaaattgacctcctatgctaaatctagggccactagtgtactaagacacctaaacctcattcttactcttgaccactcataagggtttctcgttactataacagtagatgagaacatgtataacctttcctttcctctgaatcgtccaacttggacttatatcacatctctttatgatgcataacatcttgctaacctttcttggtagcttacatttactttttatgggtataatccttttacctgtaagggcagtatccaactacacatgtcctcatctaatgtatagatttgctttaggtcacccacatagcaactaggctcatgactcttcacatgtggctcttttgctatgacctatagttaacccaccagtcccactgggctgactgtcaccttcgttaggaaaaggaagagtctctcttctattttgctctctatgaagcacccatccttcattgtagagaggttactttcccctatggtaatcagggtggtagttccaccactcacctctaggaggcctcttagccctctttcaactccttggagttgtctcatttcctttattccctttttccgctttttgtaaccacatttcatttcctcctgttaaaaccttttcatgaaaatcctttttcacaaaccttttgaaaactcatcataccttggtgacctaggggatccttagccatttttcacaccgtggtatgccgagcatgcagctcttaaacatgactgatactaggtaaagttccaccctatgtcactacggggatatcctttcaatacaaatctttcttgaaaccctaactttttttctgaaaaacctttactgaaaccctttaaggaaactctccacctttttataagagatataggtataattttaagtatcttttattttacagggttcatcgtaacatacaattccaagcatatgcaacaatcacatgatagaaaatacatatgatgctaccattatcaacaataattaacatcacaaagtaccaagcaaataaaaaatatccaaaggaaacaaggaggaaagacaattaaacaaacatagactttcagcataatcaggtttaggtagactaatacctaccttagctagcctctgtactttctcaagttcactctcaggaaggtaactggcttagccacaagccttgctatagctagtctctaaaataaattctggttactcgcctgagtttatttataatagcctctaacttagccatggattttgttgaatacaaacctaaagagaagctatgaatgtgtcgcttggtaggggtgctactggcttaattagctagctagttataactctgaaggtttgctcggtgggctagctcaatctctagcttatcgagctggtgctattctacactttgcgaaattcacaaatcaatctaagctcccaatgactacccaagcatccaaactaacaaaaattacatcaataatgatcccaatttggaagtaaccgcccttcatggagagaagcctctcctatacttcttcacgaagctatgataaaaggaacctaaacttgggcaccctcgttgtcaaacccatcatcattcattgttttcctctttgtgctttctggaataaacgagaatgtatgaactttttctaaatgcgatacgacagcgaaactatgaaaagtcaaagatcgagtgtttatttgtctatctatgtggctatttgatggtggatctgtgggttgtggtgtgatgatttttatttattggatgccatttttgctttctatagattatattacttttccccatgagtttctattgattgagaattgctttgtgttattcatgttgaacaaaatgaaatcgcacaaaggagatgggacattttggggaaaatcataaaaatatcgtgctcattagtgaaaagaaagagttttttttggtgaaaaaataaagagtttatgtaattagtgagaatctcggtattttttaacttaatagaatttttattattttttaatagattaatgtcgacatatattcattcaaacttatatcttttataagttttttaaaatctaagacgcctttgtaatttttaacacatggttgcttgacctggttttagtaaatgaattttgatttttgattgaacaaaccatattaaatttataatgcagccaaagcatccgaaaggtccccttaaaaatcagttcataaggggtgatctatccagttatataagcacttatcataattatcagatgtaggacttttcttaacatttcctcattagctcttgacggatctgttggagaagagatgtaattgtacctaatacaagaaaatcaagatcaaagcatggagacagctgtgtcgtccgcactacttagaaagctagtggattacatccgttacaggcgaaacattgatgaagtagacgagtgtgttaatttccttacttgaggaacgatgatggccacaaaaatacacggttttccaattgtttatttccttactttaagcatagactaggcagacttgcaagtctgctgaaattgcctattttgtctaatactgaccttatggattttttggttctagaaaatccataatggcagcccttgaatattccactgtgaacattattggagtgcatatggaccaggttggctggataagagaactttaatcaaagcaattgctgaccaggtggggtgggccagctttaaactatcattttttctttaaaaaaagttatgtagacctatatgcaggtttccttcactatcaaaagtttagatcaacttacatgattatattcactggtttcaaatttaaattttaaatatataattatgttaaatatttgaataaaaaattttatgatacttgattagaatataattaattatctttttagtagaaaatacatttggtctaaaaaaaccaactttccttctcagaacctggtatgtaaatgcgttcggtttaggagcagaaaaggttatagagaaggacatgacagccccaaaaaacactcagtatacaattttatttgtaatttttgataactaaaaagaagaagaaaaaaactacttctaatggaggagacgcgtttcataatctagatatttcatcaccaattaataatgcaatgaggacaagataacaattgagacatgcagagatggatcattcatttgtaaagttaaagtgaaagcatctttaaacccgtaaaagaaaaagcgaggaccacttctgttccccattatatgaacctacttccccacttatttcgtttacgtccttagcatgaatatatagattgttggggaaaatcgaggaaagtaaagtcgaagtaaacacgggggccggaagtcattagaataagagactcattaggccataagttttcactcaggtgaattagagccgcacacaaaacaacttaataatgcattttaacttaaaacctagagacttaatttataggtttattttttatgatgtttaacttttttttatttttatttaaaatgagactttatctcacacttaaccagataatcaccacaataagatagcaattgagataagagggaatgaacctttatttataaaaagctcgtttgaataaattttttcataaagatttttaagagaataaaataagaaaaataaacaaatattttaaagttaaaattagcttttacataatttaaaaaatcatgttttaaataagttaatatgaatttttacaaattaattttatactataaaaaaattatgaaataaattattcaaacatggtgtaaagataaacttaagataactcaaacagggaggatcatttctgatcgagtatgttttctggtttctttcgtcatgctctgagtattctcttgctctttaacttgatcttggttttcttcggactagtagtacgccatccatatgcttaaatcatgtttagcttgccaacatatttcagctttcttcctaaatctgctctttttttctcacattcagttttattgcttacgatttggaatagtgacaagtaaagacaaatcacaactcacggtcatccaattccaagaatagaggtttgttcctttctttatagcttattatattgcctacttcttttgttcatttatttaacatttgaagaaatttcactcataaagatggatatataaagttctcgtttagatctttcaactctcatggctaattttaccaaaaaataatatattataatttaagcctcgatttaatttgtagctctttaacaaaattttgacaaaattaattcatttcatattcaaatgtccaataataagctttacgaggcagcagcttcaaaaggaggggaatattcagaggaaaagaaagagtagttgttgaacaacaagagctttcctcattcttagtttatctcacaaagaaggtacgtaagatccttaacacccttaccacagttacttgccgcctcaatccattgctgaaatgaatgctcataaataaatatatttgcaggctacgaatctctgctcctagatttttgtttcttaattatcaatctagtctctaaattatattaaaagatttaatttgattccaaatttttaaataatttaatttaatttctatattattttaaaatagattaatttgaccatttttgtccaattgtgattagtataacaaatttcagcggcttttatttatgaacagtggaattattatatttattattagtttaaaaaataaaacaattttcaattctttttcttcttcccttttgctttgtttcatcctcgaaaggcggtggtgtagggtgttgtataggtggattagatttcaacttttgagtgagtggattaaggtggtggtggtgatggggttggagtgcaggtgttaggggttggatggtaaatagcgaaagtgtggagaaaaaaaaaatgagaattattttattttttgaaataataataaatatagtgaccgatgaagaagaaatgagaatcattttattttttaaaacaataatacatataattatggtaaaaactcgtcactgttttgtaaacaaaatcagttgaaatctgctatcttaccgaccaacctaattagatggaaaaaaagcatattggtctgttttatctgttttacaataatttgtgactaaagtaaacttttaaaaaatttggaaataaattaaaccttttaaaaaataatttagtaaccaagttaataattaagttttttttaaggatgctccaaacttttgaatttatagtgacgtatattttcattatttaactatatgctttagaatatacagtttattagacctataaccgtattccaatttaggctttttttacctaacagtggtagtttttttgaaaaatctatcaacatgagtaatttttgaatttattacggagttatgaataagttatgttttaaaacacaacttttaataaaccacatgctaatgtgcacaaaataaattaaatgcatggataaatgcactgtcatcgtgatctcaaaatctgaattccaactaatgctttctaaaactaattaatgacaatttacaggactttttatgctaaaaatttgccaagcaatatattcagattcttatagcacagtggtagacattgaatttgaagatatagagataaaaaaaaaaaagataagcttataagtttttcctgaagtcattttgatctcttcttggggttagtttgttccataaagaacatcctttgtcttcttcaaccctagccatatagaattacttttggcgggacatgggagaacaataagaattgtctttgggaaattaaattctgaaaaaaagggagtactgtaaataaattcgggaatatattttctagaaagagggtatgatttaaaagaaaaagtgaaaggggagtgtattagcaagaagggaaagggggtaaatagcaactgcctcccaagttacatcttgttagaattcataattttgttggtgttactgggaacaaaagcaaatatttttgctacggaccaaaatatacattcaaacttggtatctttttcatatttttttcaaatttaacattcatttataatttttaacacatgatttcttgatacaattttagtaaatgcactttgattgagctattgttccaaatctggtaggaggtgtcacgtgagaattgtttttgacgaaccagtcaacacaattgttctcttctctaagagtatggttgaaaaaaaatatatttaagcttgacttatccattttgagcatctttgtcttacaattgtattgtgatcatcaacatatatggtagcagtcatttggataaattattttttagcgtcctggtcttgcaattgtatagcgatcatcaaaatatatttgaaattattgaattctatttaattaaattatatttatcatttaaaaatattattgaaaatgaaagttttaaaaccatggagtccttcccgttgttcattgattattaaatgaacttgtgaattttgattgatgaaatgtattttcttaattggttctagactgatcttatttatttatttatgtggggaagagatgtagtcatagtacaagaaaatcaagctgaaagaatggagtctgtgttgtccccgatagtggaacgaacgtttaattttgtggtggaccccattattcggcagttggcctacatcctccgttgcagtcaaaacgttgatgaactcttgactagttttaagagtcttgaacttgaaaaagagtccatagatcgtcgatgcgaccaggctgagaataatttacaaaacattgaagccaaagtcaaagtatggtcaaggaaagtagatgagttcaagactgaactagagaagttttggaacgatgaaggccacagaaagacagggttgtccaatgttttatttttatttccttacttttggaataggcatagacttggcagacaagctaagaagatggcagagattgtcaagaatctaatagatgagtcggccaagttcaatgaagtttcctactcagacaacttaacatctaatgatttcactttgtctaatccgggctacatgggatttgcttctagacattctacggtggaaaaaataattgcaaaacttgaagattcctctgtaagaatgattgggctgcatgggagtggtggcatgggtaagaccactttaatcaaagcaattgctaagaaagctatggacaagaagctgtttaatgtggtggctgtatcagaaatcacagccaatcccaatccacaaaaaatccaggaagacattgcttctgcgttaagattgagattggaaggggagggtgagaatgctagagctcatcgtctgatgacgaggttaaagcaagagaaggagaacacccttctaatcttggatgacctttgggacagattagacttgaataagttagggattccacttgatggtgatgttgatgataatgatttaaacacgaagacaagcaatgctaaactgggtcccaaagaggcgactaaagaaaaatctcttggtgattataagggctgcaaaattttgctaacttcaagggataaaaacgtattaactgataaaatggaagttaagtcaactttctgtgtagaggaattagatgacgatgatgctctgaggttgtttcggaaggaggctagaatacagggtgaaatgtcccagtggaaacaagaaattgttaagaagtattgtgctgggttacctatggcaatagttacagttggaagggcattaagagacaagagcgactcagagtgggaaaaacttaaaaaccaagacctggtgggaattcagaattctatggagatttctgtaaaaatgagttatgaccgtctagaaaatgaggagctcaagtccattttctttctttgtgctcaaatgggtcatcaacccctaattatggacttggtgaagtattgctttggtttgggaatacttgaaggggtctactcacttggggaagctcggagcaaaatatctacatcaatccaaaagctgaaagactcaggtttggtgttgtatgaaagtcctagtattaatttcaatatgcatgatctggttcgagatgctgctttatctatagcacggaaggagcaaaatgtatttactttgagaaatgggaaacttgatgattggcctgaactcgagaggtgcacttctatttctatatgcaacagtgatatcattgatgagcttccagaagaaataaattgtcctcaacttaaatttttccaaattgacagccatgattcatctttaaaaatacccgacagtttttttaaaggaatgaaaaagctcaaagtattaatgttgactggtattcaactatcaagcttaccatcttcaattgaaagcctatcagacctcagattgctttgtttggagagatgcactctagatcacaagttatccatcatagggaagttgaaaaaattaagaattctcagcttttctggatctcgaattgaaaatttgccagctgagttaaaggacttggataaactacaattactagacatcagcaattgttcagtagtcaagaggattccgcctaagcttatgtcaaggttgacttcgttggaagagctgtatgtaagaaagagcttcatcgaagtgtcggtggaaggagatagaaaccaatgtcaaatttcatttctttccgaactaaagcatttgcatcaattgcatgtggtggacttaagcattccatgtgctcaagtttttcccaaggaattgttctttgacaagttaaatgattataagattgagattgggaacttcaaaacgctttcagctggagatttcagaatgcctaataagtatgaaaagttcaaatctttggcattggagctgaaggatgacactgacaatattcactctcagaaaggaataaagttgttgtttaaaagagttgaaaatttgttgttgggagagctgaatggtgttcaagatgttattaatgagttgaatttggatggatttccacatctgaaacacttttccatcataaacaacgctagcatcaaatatatcatcaactcaaaggatttgttttatcctcaggatgtttttcccaagttggaatctctatgtctctacgaactaagaaagatagagatgatatactttagttcaggtacagagatgatatgctttagtccatttacagattgctcattcaccaaattaaaaaccatcaaggtcgagaagtgtgagcaattgaagaatcttttctccttttgcatggttaaattgcttgcgagtcttgaaacaattggtgtttccaattgtggttctttagaggagatcattaaaatacctgacaattctgataagattgagtttcttaagttgatgtctttgtcacttgaatcattatcatcattcactagtttttataccacagtagaggggtattctacaaacagggatcagatacaaattactgttatgactcctcctctttttggtgaactggtatgatagtgcatatttttcctttaaattttttaattagtttgtgactaattatttttgagttgtaaatatgttaattgtgtatgtgcttgcaggttgaaataccaaacttagagaacttgaatttaatctcaatgaacaagatccagaagatatggagcgaccagcccccgtcaaacttctgctttcaaaacttaataaaattagttgtgaaagattgtgaaaatttgagatatttgtgttcattgtccgtggccagcagtttgaggaaactgaaaggcctctttgtaagcaactgtaaaatgatggagaagatttttagcacagaaggaaatagtgcagacaaggtgtgtataggtgtatataagtaaagttttatagttgactgaatgttaggcatttctgtttagtttcattaccaaataatggcttgttttcatcaaagtgcaggtctgcgtctttcctaggttggaggaaatccacctcgacgacatggatatgttaactgacatatggcatgttgaagtgagcgctgattccttttctagtctcacttctgtgaacattaaaagttgcaataaactagacaaaatttttccgagtcacatggaaggatggtttgcgagtttgaacagcttgaaggtttcttattgtatgtcagtggaagtgatttttgaaatcaaagattctcagcaagcagatgcatctggtgggatagacacaaatttgcaggttgttcatgtatcctgtctcccaaagttggagcaggtgtggagcagggatccaggaggaattcttaacttcaaaaaactgcagagtatagagatgtttcgttgtggaagactgaggaatgtatttccagcttctgtggccaaagatgttccaaagcttgaatacatgtcggtcatatggtgtgatggaattgtggaaattgttgcctgtgaagatggatccgaaacaaacactgaacaattagtgtttcctgaactaacctacatgtgtttaggttgcctatcaagcatccagcatttctacagggggagacatcctatagagtgtccaaaattgaagaagttggaagtagggaaatgtaacgagaagctaaaaacattcggaaccggagaaaggagcaatgaagaagatgaagcagttatgtcagctgaaaaggtaagtcatatagtgagacataaatggagagtgatattgtaagatgtgtggcatacttaaaaaagatatagccctgctaaatataatatggattattaatttaacgtctgtggtcccctaatccctcttttatttagaattgaagggaatgtttgattaattattataatttaagagcattttttagtcttgatgggaccccagagtaaccaatgtgctccatcagggataagcttccttcttcatagtttactatttacttttgttttatttttctgtagatattccccaacttggagtgtttgtatattgactttgacgaagcacagaagtggttattgagcaacactgtgaagcatccaatgcaccgtttaaaagagcttaacttgtactatgttaatgatgatgaacgtctctgtcagattctgtgcagaatgccaaatctagaaaagttatacttgagggaggatgaacatttgcttaaagagtcgtcggagtcacgtttgggaaccgtattacagctgaaggaattagttttgtggtggtcaaagataaaggatataggatttgaacgagaaccagttctacagagactagagcttttgagcttacattggtgccacaaattgaggaatttggctcctccctcggtatcattggcttacatgacaaatttgaaagtagggtattgtaaaggattaaggaatttaatggcatcctcaacggcaaaaagcttggttcaacttaagtccatgaagataagccattgtaataaattagaggaaatagtaagcgatgagggaaatgaagaagcagagcaaatagtgtttggcaaattgattactatagaacttgagtacctaccgaagctgaaaagtttttgcagctacaagaagtgtgaattcaaattcccgtcattggaaggattgattgtgagagaatgcccaatgatgcatacattcacggagggtggcgcaagagcagcaaagttacaaaacatagttactgctaatgaagaaggaaaagaggaagccaaatggcagtgggaaggagacttgaatgccaccatacaaaaaggtttcaacaaggtacttattcatttatttatgatttaaatatatttttatttatcttgttagtattttttttattcgtcctttttatttttgttcttcaaagttattatttttgttaattttagtcttttaaagatatttcattcatttttaatacgttaatttttttttaattttagtctctttaatattttaaaatattcatttataatcctccgtgcgaaattaaaattaaaaaaaaaatataaacttatcaaataaaaaatgagtaaaacatattacggagatcaaaattaaaaaaatataaactttcaacgactaaaaataaaaaaatattaacttacatttatcaaatacatatttaaacttatatttattacaaattctcctagttttattaacttttcttctttccttaaaaaaacaaatttttcttctttcatgagataaaaaaaattacttgccatgaatggaaattataaatccttgtttttgccataaaaagccttaaccagcaagtatgttaaatttcttaagatataaatatgttagttcaaaaaaccagcatttatataaatttattaatcatttttgttttaaaaaaattcatttataaaatttaaaataatatgaatttgactgcttcatgtagccagcgtagtcataaagaaaactttccctcatcttacaacttttctttttaatcaaaataaataaacttcaattcatatttgattgtcatccattaattttttgttaacatttttactccttctagaaatttttgctcattattaatcctattgtttatgttatatttaagaaaaaaaacgactaaaaatggatcaacaaacacatttttagagggattaaaactttaacattgaaaattgtaaaaaaaataattgaggggaaaaattagaatataaaaatatgaggagttaaaaacttaattaaaaaaaattaatttgttgatttgtgaaatggaacaagttgcatttgactttacatatataaaataacatataaacgtgataataagttaacaaatttatccatataatttgtgactatttatatatctataaaataaaagtcacatgagaagttattataacttacaaggttgattttgtagttgggaagaaggatttgaagggaaaaggatgaaagaggatttaagttacgatactttcactaatattttaataaaaaaactaacaactaatattaattgatgaacaaaatgtttttatacatactgaaaaataaggataaaatgtattttttttctttttttgttaaaatataaaatatcgtgaaaataaaatgacattattacacctaagagtaaatgataaaatatttcttaaacgaatcatccataatttcatcattgataaaaataaattatcatgttatttacttatatatattttgtttaaccttttgtgcagctatccatatttagtattttagcacatgaatggcagaaagtaaatatttatttatacagcaagaatttgtaatttataataaataaatattttaagtatataaattatattataattaaattttctaatatataatttttaacatataaattacattttaaatttatgaaaaataatttatgttaggatgtactattattttaaataaaaatatttaaattaagttagaaacaaggatgccgtcaagttttcttaggccaattatcttagttttgtttctcttgatggaatgaaggtgttttaatgtcccctgatgatgaaacttggaatgtaacaaccaacataattgctccctccatattaatgtttgtttgccacacaactcagtcccatcattctttggcattggcttcttcatcttataccatggattcgttttttattattaattattaattatataaaaatttgtttatatttattatcatttgactagtgagttttaagatattaggtgtttttcattaaaatataataaatatatttttaattaatatgataatctcaaagtaaaaaattattatatttattattaaaaaaatttcattgaaagtttaaaataatgttaacaactaaaaattctcacttgaattaatttaatcatgtcatccacatgtgaattatttatatatataatttaataaataagattaattagttttcttcaataaaaacagttatggatacattaatctgtctccattattattatcttatttcataataatcttatgattaaaaataaattagattaaaattataaaaaaaattaattattattattattataatttctatcatgtaacatgtgtttaatttcaacctagatctatcaagttaacaatttaataatacataattaataatatgatggattacatcttttaacatacatatttaattctaacaatatatatatatatatatatatatatatatatatatatatatatatatatatatatatatatatatatatatatatatataagttaagtagcattgataaagtacatagaaaataaattaaattatttaaatattatgagtttaacgtaaaaaattaatatttgccaaattggattggcacataaatgttgtttctgtgagttttttggttttccgaaaaattggacgctaagataagttggatgaggccaaagttaatttgaagatgaatctaattaacctaacgctgtatattttgcattataacaacatacaccaatatcaatcatatttcatgaaaataattgaaaacataccttttcccaaaatgcctcccgcatggtagacttcaggtcattttccaatttgatagcatctgatgactcctcaagttgaacttgaaccaacttgtctgctttaagggcacctggacataatgtttcccatccactcgcaatagattactgacaattcctccaatgaggggaaacttaataaacttcctttatagaagcttctcagctttggtaaacatttaagtttcaaacaattgagctgcggaaatattatttcatcctcatgtgattcccctccctccttagagactacctcttcaattgaatcacaccattctatctccattcttttgagtcgagtcaaacttttggctgttgaggttgtcaacaaatatagtaggcaattgcatctttctactttcaaatatgtcagattggaaaaagacactgtagatggtaccaagtcttttaaactagaacaacctattacttccaaggtttctagatttcccattaagggctgaatcgaagagttctctaacccaatggaaacaagctctggaagggagtccaagcataagactttgagctgtaataggagtccagcctcatccacattaagactatcaaagcagaaaatctccttgaaggaaccatcacacaccacaagcttctttatattgggcgccagttgtagaatttcatatggaaatacattcgacccaatatgaaagcacagagtaagaacttgtaacttgtgtatgaagtttctctgaaattctccacgcttgatcatctccagtcctttttcaccgagtgacaggcactgtaagttggatgtaggctaccaagagaaanccgataatccaagcaaaagcaggttagtgaggttataaaagagatgaacctggctagctagctattaataaggtggaagggattttgatacggtttgtgcctaaacaaatttatttatttgtagtttaaaattttagatggaaaaggtgataaaggagaccttaagggaggtagtagtggttccaacggagaagaaaatgatgaaaaattcgatcctttagatggtagggagattgtttataaagatttgaaaaccatccatacatgatatttgataaagtaaaatggcgagtgtaagattctatgtaattgtctgtattaataagaataaagctcaaacctaaaatgtcattgttgtgtcatgaatgcaacatgtacccttaagtgtattcttttacttatttatgtaaaacatgaaaaatgggagttaataggtatagaagatagaacacggcatatatatatagcaatgtacgtgaaattctcaaattttaattttaagagaatgttcatatcactcatgttgaacaataaaaatgaagtagtgcaattatatttaatttgcaggcatgagattatgcagtaaggacaaagtagtaagacaatagtaccatttcatccttggtaggtgtctggaacatgtcacactgcagtgagcagtagtaaaaatacttgaacttgggcaaaccttgtagtttcaatgacctcacacagggacccgggaacgtaagctccagatttgcttctcttggatctgcattatcctctgcaacaatttctatcaatccctcacagtctttgacaactagtttttcaagatctttggctaatgatgcgggaaacacacttgtaaggcatttacatttctcaacaataacatgttgtagaagttgcatggttagaattccatgaggatcttcattccagacattctccagttttggcagcctctctaaagtcaatttcttgagggaaaaagggagaggtctagggaaggctgctgctcccaatcccgtagttgttttcacgtcaaatatgcttttcacagaacgacagtttcggacttgtaattcttgcaaattagttaagaaaggaagtaaatagaagggtatgacaacatctgttaaaaattggcatccgtccacagttaaagagttcaagttactgaagcacgactttgggatccgccgtgagtcaagccatatcacttgtagtgggctatctatgagactaagagatgatgcagtacttgcagactccaaaaactgtgcataaatttacaaaaggctaatatgtcagtttcatagctcgcgattacattataacaaatagatagattagtcataaattagttactgtattaatactgaatttagttaataaaaagacaaagaaaaagagaggaacttgatattagacatagagacgcatgaaatcagtggcgggactttgagaaaaaaaaatattggaggaaccaaaatattttttatatagtataattttttttacttttgtctgaaaaattaaaaatttcatttccattttttcgtgtgaaaaaatgaacctgaaacatttagtttgtttgtttatatacaaaatatttgtgtgaggatacagggaaaaaatctatggattttttaatgaacttaaagaaaataatcatatttttctctctcaaaattataacaatttgtagaaaaaaatcatagaaaaatctgaataaaaatgggtttaacaaatgaaaaaaatagctctatattatataaaatattattaattaataaagaaagctatttataatataatctataagatcatagatttattttattatttttagataaaccataagtaaaaatgtaaaattatatatatatatatatatatatatatatatatatatatatatatatatatatatatatatatatatagtatatagaaatatatatatatatatatatatatatatatatatatatatatatatatatatatatatatatatatatatatatatatatatatatatatatatatatatatatatatatatatatatatatatatatatatatatatatatatatatatatatatatatatatatatatatatatatactatatatatatatatatatatatatatatatatatatatatatatatatatatatatatatatatatatatatatatttatttatatatattattattaatataaaatgaatccaaactctttcaagtcattgtaaaaatgatttgaaaattatttaatttttattttattagtaaatcatttaattttatttatattaatcctattaaaataaaaaaactatcaatcgtttataatccactcttatatacatattcaaaatatttatcttttaacaattttaactatataaaaaaattatcttctaggattaagatactatataattatatttaaaatctagattaggtagttaaaatgttagttgttcattttttttaattttctttaactttttgttattttttcatttatacgtaaaaataaattaaataaatattaaaaaaattagagtgacttgaagaaatttagattcactgtatattattattaattactgatacaaatgaaagttaagtagtataagtaaggcatataaaaaataattatttaaatattagtgagtttagcttactaaaaaattaagagattctttttttgagtataagataagagattaatataagtcaaataattttgtttgtcaacttagcagcatgacgtaactgcaagctcaatctaatggtatatattgtgttggagagaaaagtatgttcgagagcatcgggatcttatattttgacggagattaaatgtgttgtatttattagtaatcattgcatatatcaatttttttataaggaatgatcattgaatttgtgcgattttcttataaataacttgtcagttgagaccaataaatatgttaattttcttttaatttttctaagtaagataaaaaaaatactcgatctactatctagctttgatcatgcgtatgttttgtcgaatgattaattttgaattttgaaaaataatctactctttctaatacaaatatctagaaacttttttaatgagtgcaacaaaggcatataaaaaatattttggaaaatactttcattgtgtgtgtttgatgataaatttagaatgaatgtactagaatcaactagttgaaaactgttatctgaggcgcattcctccagaaatcaactcaaggaggagctgtttttttttcctttacaaattatttaaataactttgaaggcagattttgattagagagcatccacgattaacggaaatctagactataatcagaaatttgttactttttttaatttattattttaaaaattgagagcacattagttatttggtgcgattggggatgcatttcttcctacacaataatactactcttttttttattcttttacctttgcagttatggaagtctgcagataggagattcaatattgacctcaaagatagccgactacaagagatatggcttaggcttcactcactgcatatccccccacacttctgcttccctaagttacacaccttgattgtggacggctgccattttttatcagatgcggtcttacccttctctttacttcctttattacctgaattgaaaacattggaagttcgaaactgtgattttgtgaaaatcatatttgatgtgacaactatgggaccactcccttttgccctcaagaatttgattttagagcggctgccaaatcttgagaatgtttggaattcaaatgttgagcttacgttcccccaagtcaagtcattggcactctgtgatctgccaaagttaaagtatgacatcttgaagccatttacacatcaagaaccgcatgctctaaatcaagtctgtattcaaaaggtatcattactacttctatatgaactaaattgccatcattagtgttctttattacttctatatatatgaaccaattgccatcctgttaaaattaaaaagttgtgaacctttcccttatacagttatatatgctatgttgaatcttctatccatcttctacgcatattggcttccattttttttatgatttatctacataagtcagataatacagctactattcaatattattgatcttgatgaatttataatggaacatgttgcattcatgatgtgaaacagcatttgaggtacgatccttattctcgttattttcttctgcaaccttactaataatctcatttatcacttttcccgtctttttttcttttttggtttttaagtaaactgatttcttttaaattgacttgtttgcacctatttaattgaattggttaatttgtcattaattattggacttattttagcgtgaaagttcattatttcaattatataaaaagagctggatctataactgccctaacctgctttttcttcattgttggtttctcttcgtagcttacacccaacatagagcacctgacactcggtcaacatgaactcaacatgattttgagtggagaattccagggaaaccacttaaacaagttaaaagttcttgctctgttctttcatattgagtccgatgtatttgtacaacgggtgcccaatatagagaagcttgaggtgcttggtggtttcttcagagagattttctgctttgatagtcttaatgtggatgaggcgggattgctttcacagctgaaagtgatatgctcggactcccttccagagcttgtttccattgggtcagagaactctggcattgtgccctttctcagaaatctagaaacattgcaagtaatcagctgtttgagttcaataaatctggtaccatgcacagtgtctttctccaatctgacatatttgaaagtaaaaagttgcaagagtctgctatatttgttcacatcctcaacagcaagaactttgggtcaactcaaaacaatggagataagttggtgtgattcaattgaagagatagtgtcttcaacagaggaaggggatgaatcagatgagaatgagataatatttcagcagctcaattgtttggtacttaaagaattaggaaagctgagaaggttctacaaagggagtttaagtttcccgtccttggaggaattcacagtaagggattgcgagaggatggaaagtttgtgtgcaggtacagtcaaaacagacaacctgttagaagtcacatttgattgggatgatatcccattggaaactgatctcaactctgccatgcaaaaccgataggcatttctggcaagggtatgtctttattttttttattcaacattattataattgtttttatcatatacactgtcaaaaatttaaatcttgaaaagagtttaattgccgtgtttaatttataattaaattctaaaattttctggtcggcttgtcattgtcgtcattattattattaactaacatattttttatgaattgcttaggcaatgactccaatattgcaggtaaaggagattgattcaccaaaaagaaaatggttagagaagaagagatcaacaaattaagttgcccagtgcctaccgaaataaaaaaataaaaaaaaacaaagtggcccggttttattatttttgtttgtgtgtgaataaaaaacgatttgtatttgtggtttacccagttagtggtcaaatacactgttctgttatatttattatgtaattaatgtgtgtgatcataagtcataaacgaccatggaaaataaaataatacatgttcggtttgtttaccattcatttcaagcttcaaagtcttcaaaataattcttaaaaaaagtcttcaacataagtttgattgtagtgggtgtttatcactttgcccatctcctaccattaacatctaaaattgttagctttacccaaggccaataatacaatagaatacaaatctatcaatttgttctaaatcaaatcttaagtagtttaacactctaggtttcacatattccaacaatacaatagaatacaactttgcatcttaaaagtgatataattttagtcttttcattaaaaaaattaaagcacacgtctctgcatgttaaaatataaaagttaaaaatgaatgatttttttaaataaaataaaattagtattattttaaaggaaaacaaaatatttaattcaaccaaactttgtagtagtcatagttaaacttttcggatttgaaattattgggattaaaccataataattctttatcatttaatctacgtggattgagatgtcatattgtcttatttcgggaataaaaaaaatgtggaaggaaaaaactaattaatataaattccttgtaactaaaaataaagtgaagaaaaaataagatatatgctatgataatattcaagtgtgttaattttaatttatctattttctatagtaattaaattttgatcaaaataattaataaattattttgatcaaaattgacaaattaattaaaagttaaaaaattaatttttttacatgataatgatattgttaattgatttattaaattacaaatatttaatttgtaaattaactgttaagattactagtaaaacataataatataaaagatatatttatgattattatataactgttatgatttttatataaatgatgacaatggcacggccccatctctagagaatgcataagccatcctgaacaaattttaagcatcacattatattcataagtaactaactagagaaactaaatcccctctcttatggaaaatgcactacgggaaggtattattattattattattattattattattattattattattattattattattattattattattattattattattattaattgaaaattgtgtcttgtgtcttaattgtttttttgttagatataagtgtaataattatccttttaaaaagttattaaataataattacttataattgggggtgttttttaaaagtaatacaagaataaattaagaataataacaaatgtatataaaaataattactttttgttaataattataaatttaccttgaattgctggtgaccgaagtcaaaccacagaaacagcaacaactaccaagcaaataatgctcccgattccattgccgtatcatctcttgcaaggcagataaaacatttcagcaatggcatatccaccaactataatatagatatcctaactttgctgattattccttttggtccatttctgtctgcaaatgcaagttaatttgtagtaaacagaaagtattctttggtctaaatcatatgagctgatgcatcatacccaattagggtatattggctgttatgaaattaaggagaataaatatcgaaaagatttacactaatgattctagagcaatcatataagtgaatctaataccacactttaacttaaaattttaaggttcaagtttatacgtttttttctcatatgatatttaactttttcatttctatccgaaatttcacctcagacttatacttcatcgcactcattagtaaaatatgttgggttttgttctttcttattgagtccaatgtatttctgcaacgggtacccaatttagagaagctcgtgatccttcgtggttccttcaaggagactttccactttccagagctgaaagtattacctttggagtcccttggagagctggcttccaatggttcagagaattcttggaagtattcatctgttttagttcaataaatttggtaccatgcagcgtgtctttctccaatttgacatatttggaagtagatagttgcaagaacatgctatatttgttcacatcctcaacagccataagtttgactcagcttaaaacaatgaaggtaaaaagctgcaattcaattgaagagatagtgtctaaggagggggatgaatcaaatggtgatgagataatatttcagcagctcaattgtttgaaacttaaaggattaccaaatctcagaagcttctacaaagggaatttaagtttcccatccttggaagaattgtcggtaatccattgcaacaggatggaaactgtgtccaggtactgtaaaaacagacaagttgttacttgttagatgttcaatttcagagattctcagatgctatcccattagaaaatgatctcaactctaccatgcggaaggaatttcagccaacggtatgtgtttatttttgtttaacattatgatcgttgttgtaaaaattaatataccttatcagtaattcaatcaatcgcttgaaaaaagtaggataattatttttattttatttttataacgctaaaaaaatactaaatctagctttgattcacgagaatgtttcctggaaagtgtttactgcttctataattaaaaaaaaaaccttcattattattataaaacagacatctgtttatataatatgtctaggctaagacagcatcattgcctaagaggaaatggaagccaaagagagtttaatgaagattaacaaggtggccttgttttgcgtttgttacatgtgtggttctactgagttgatgattaaatacattgttctattatatatattatgtaatgtgtcattataagtattaaacggtcatgaagaacaaagattgggtgatgaactctatcattaagtattgatattgactatttgactgtcttgcttcctgcactgacaaaaataccaaaaaaagtgttgtaagttatatattttatgtttgttttcccttattaatccaaaaaaaaaaggaataacctaattgatccttcttccaagtaatttgaagcacgagatcttcattagcttacatatagcatccaatattccaaagccgtcaagcttcaactgcactgctttcaaccacctatccacataatcttttaacccaaatccacggctaggtcattgccaaaatgatagcactgaaaggtagaaacatcaaacatgtgccttcacagttgacagtttttatcacctgcatacttttccagattgtgccaaaaatgtaccagaaagaatgggcctccaaggaaaggacaagatacagttgaatcgccagtgtccaattcctctatcctgtcatggaaccatatgtattaaaacactataagctttctgtagaaagagtgcaacatgcaatgagcacaacatgctcttaactaggactattacttgcaaaactaaatttaggaaaggaataaagttgtatcaaaagtttcttcacttcaaaaccttcccacttcataagctgcatgtttgagttctccagaagggatccaaaattagttcagttccttttgagtcgctgaagccctttaaatgagcctgcagggtctgcaaattcacaatcctcttcatacgcatttaatgtcagatctcctgatgattgagaaaagcatattaaccacatgaaccgtacgtctgcaatttaaccaaatgacaaattagaagataccaattggtattagtttgattttatgccagtcgttatatggaaagcaataatttggaggttatcctgtatgaaagaacatactctggtactggaagtacacttcaagtatgccccttttctgtgtagtgatataagactagcagtgataatctctctgcctgtaccaagttgcatcaggggagctagcaacaatttgtagtacaagtcatcaaaccttgaagatgcatatggattaaggrwrksaksmysskktwccswktgattaaaagaagacaagaagccaaggttacttagaagggttcaaattttataaggagggctttagtaaacactcgtaagtcatatagcattgaagaacaatggtcgtaaaagaaaatgagggactgtaccattgagcttatcaaccaatcaggatcagccaaggataaagtaattatcaaaataaattatttgctattaaattttatgaagcaaaaaaggtcacatatgttcacataaaggtcatatacaccactctcatttagggtctatttgatagagatgttagagaggagagaattaaggtgaatggaaattaaaaaaaaagtaaaaataggcgagaaaaaagtgaaaattaaatgtatttggttgaaaagaaacagaaaataagttaggagagagatagaaagatgaaaaaaaatatgtgacaatagtatagtaagtctcatcatttttaaaattattttcatctttccatcctccgttctcccccttatttctcactgtctcgcatgccaaacatgggttcaagtatccctcaaaattttccttaggtcagccctgcaaccaaatagaccactactatcatctttgctaaaaaatgtaaccttcacttgctaaaaaatattcttaatttgcatgctatatacaaaggagggagatcaattagattactcaagggtattcatcaatgagcgacaatcacttataatctcaataaaattcatacttatcacagaggcttaagctttcgtgaagttaaagatttgtcaatcaaacgtgttgagaattcaaatgtgaagcagagttgtgttaaaatttgtgcttatcacataatttaagattttgacttaagacgtaatgtcataatttcaaattaagtgtatgaatctactcaagagtttaggactcccatgacctataaaaacaaatctataattctattaataaaacctatcattcatgatgcatcaaattggtctctcttactcaagggtattcggattagttccactgcatcgaaacctgtggattctaaggtgctgcagcttctgttgtttcggtggaaaaatcaaagagctatggtgacttctgttgaatgaagtcaaggcagttaatgccacgacacaacacaacacaagacagtgtgagagaatcaaagtggcagttgctagttgttacctacggtttgtttaaatatctaagcttcaaactaatgagagccatacaaaaactaaagttttttttttttttttctggtcgcgggaatcaccaaccaagcatagttccaagttaaatttcaactattttaatcaactcgtattttagcatgtaaaatataatatagaagataaagatatttatttttaagattatcagaaatagattgagtctccagccctcaacatgttggtgcgtgtaggcgaaacgatcaaaacgggacaagtaggagtaaattttaaattaacaccaaaaaatgagtaaatcctaagatatttcacgatatacaaaatggtaaactaatttaagactttgatttttttctattaaaatcataaaaaatctatgaattcttattttttaattacgagtttgaagatataaatattttaattttttgtacaaaattattagaagttataaattatttataattttaaagttataagttttttatcaaaattaattattaaaaatttaaattatttttcatttttaatttgtaatcaatgtttttaatattttttaactaatattagaaaataatattaacttaaaatttattaaataatattaaattatttttaaaaaataattaatttttattgttatcattaaataaattaataatttcttacattattattagttaattttatttgatatattatattaatattaacaatagaatatattttagtaatagaaatagttgttaacgtaaaaataacataatacattcatataaaatgaacaatataaattaaacacaaacataaaattaaaaagtagaagtaatattagtcttcaacttcttgtatagaaatatactataattatgttctttaattttacacaatgattatttcctgggtttatttgaaatagattcgtctatcttatcaagtatagactagtagtgagtcttcacagtcctcaacgacaacaatgcgtacaagtgactatgtgtgattaccatagaatgaaagatctcggtacacattcaaaagaactcgttcagaaatatcatctcgttattaatattttcttaaacaaatatgtgtagtaaattttaatatttttatttaaaattaagtcttttgcattttttttaatttacaaaacaaaaaataatccttgggtaagaaaatatataaaatatattaaataaaataacattaaaaaatatagaaaatattaaataaaaaacaatatagtatggaaaatattaaataaaaatataaaaaatattaaataaaattaaaaatgaaaactaatttaaattcatttataattaattttaataaaaaaattacgactttcaaaatcataaataatttaaaaatttaaattaatgaataatttaggtgttgtaataaaaattaaaagaaattaaatttatgactttaaaatcgtaattaaaaaatagtaaaaaaaaataaagttatatattcagaagtcataaaatatccttaccattttctggttatcaatttaaaatttatcctcaactgataatttacaaaatttgtgttccttcttttggtcgttttgcgcatgcgcatatatataaaaatcatatatataaatatcatgtttaaataccccaagttcctaatatatgcttttgttaatactaacacggtgacacctatttcatttttttattatgttcaacgaaatattaattatacaaactattttttgaaaataatagcaaataaacttaattaacatcttaattaatataaaattaactactaattacataaatcagttaccataaagttgtttcaacttaatttgtgatctagataaaaaaaaaaaaaaaaagcttaaaattaaaaataaaaaaatcttatagtatttgagtatgaaaggagtcctcgtgaagcatagaaaattgaagaggcaggggaaggttagtcaaaacattggaatttccacacttgaacattgaggatgacacaactcaacacccacaaagacaaggtcacaaaaaactcatgaaacgatgtgcttgcccactagcctctttacttctcctagcaatagtaatcatagttttaatcttcacagtatttcgtgtcaagggccccatgatcacaatgaacagcgtcaagatcacaaagctccaactggtcaacaccaccatgacacctcaacccgcagccaacatgtccttagttgcatcgtttaggtatggcaacaccaccacgagtttgtactaccatggtgtcttggtgggggagcctagaggaccacctgggagggccaaggccagaagaacattgaggatgaatgtcacaattgatgtcatcacggatcgcgtcgtttcttgggcaggtgaagatcttgaacttgatatttccacacaggctattaaggagcagagttgtaagcggaaggttaaactttagtgatcatgtgtaatgtgttaggttagtttctttcctccactctgtatcatccatccaaattactatattagtttagtttatcttagtttccttgaatatacttgcaaaattactgtaccaaaaataatgagtgtttactttcttctaaacaaaagtcaaattgatcaaaatccctctatttgggggcgattatatttttaagagtcgagatttcatattaaaacaaatacaatttacgtgtaaaaagattatgttttagacttagtttgaaggtatccaactcaactattctcaatgttttgttttatttttgggtaagtcttgtgttcctcatctttagctaaggggatgaaatagaatgagagaatttatgaatcggttgtggtgtattattcgaagaataatgctacgaattttcataaaagtggagtgttgcctaatttggaaccatgcacagtgcctttctccaatttgacatttttggaagtaaaaaattgcaacactgctatatttgttcacatcctcaacacccaaaattttgggtcaattcaaaaaaaaaaaggagataagttggtgtgattcaattgaagagatagtctctaaggaaggggatgaatcacatgagaatgagaaaatatttcagcatctcaattgtttgaaacttaaagaattaccaaagctcagaagggtattatgtcactaaaggattgtgataggatggaaactttctggcaagggtatgtctttttattcaacattatgataattgttgttataatacaccgtgtcagaaatttagttttacgagtatgttatgtgacaagattaatttaaattttgaaaagaatttaattatcatatttataattaaattcaaaaattttctatctttcgttatctttgtcatagtcgtcattttattattattaactaacgtaacttttataaattatttaggcaatgactccaatattgcagttagagaagattgattcaccaaaaaaaatggttagaggagaatgatgacaaagagagttgacggagatgaacaaatcaagtggcccagtgcctagagaaaaaaaaaatttaccaacttggctttgaccaatttgcacgctcatttgactcttacgaattttcattttaaaaacttgatgttgtgactttgtaatgtcattcatattaggaaaatcatcctcttgcatacaaattttctctccgtttctacaactattagtagtatactattgcactctcaataaacttgagaaaataaattttaaacatatatatacaacgtattgagcaaaagaaagattttttttaaaaatgataatcgatgtggtaagaaaaacataaaaaatattataaatataaaaatgatataagaggatcaaagttaagattattaaaagaaaaaattgatgatgtgattaatttagctcttctagatgaaaataaatttcaattattgatgaaaaaaattaattgttggtctggcgactggttgtgttttttctacttaaacctctcgacatattattttcaatctaaatatgaggatatttaagttagaattgtaatatagaataattttaaagattaaaagtgagaaatttagtagaaatttaataaaattataagatataagataaaaaaaatttaacagtaaagatatgattaaggtttgaaaagaaaatcaaaataataaaaatatatatattaaaagaaaatttaaaaaatatattttataagtaattaacgaataggagtggtaaattataaacacataatattattattaataataataataataataacaataataattagtcaaatctattattaacgtttggatatatttatcgtattttttatactttcaaggattaaattttatattttcatctttcaaatgcgcatttatcagcggggtaaggagacgaaaagtcaaaaaaatattatgataaatatgtccctatgctggcaatccaggttaacaatcattttagtgacaaatttgtcctttcgtgtcgcgtagactattttattaacggtggcggacgaaagttaacggcatgatatatttatcacactttttacatttttcggtctaaattttatatttttatcttttaataatatatttgtcagcaaattacacattaaggaacaaaagtaactatttatctattaacaatgtatgaatttaaagttcacatgaactgtttgattaaatttacccgtaaaaacgaggtgataatttggacgtgcacataataataacatcgtatttacaactacaattaggagatgttggtaggagattttttttaagtttttcagagaatcctaaattcctacatttgatatgtatattttaaaaattatttctagaaatctaatattcccatggataattttttaattgagtttttcacgaaacatttcgataaaagaagatacaaatttaactttttaagttaaaaaagaattttacggtgataaatataattaaaaaatttcatattactcttattagatcatttaatttgataaacatttaaatttttggaataaaaacatcacattaatatttgatttccaaaaatattgtataactttttctagacatatttatgaaaaaccaaatattttttaatcatatcctaatcataatttttaagatttatgatttcctgattaaaaaaaaacatttttcttttgacagaagcaagttttcatgagtgattggcagtgctggcacatagtcatgtgacaactcaaggatttataacattcttttgagttaagttcagtcatggcctcccatgcaatttggtcatgacatgcaaacttcagaaaaataaattttgctagcctctgttttaaatattgactgccataggactgttttgtactagttttatgtcttcttgtaactaatagtttatattattctgactaaaactggcagatcggaaatgttttaagtggcatgggtattaatattaactatacggatccatgttaatcatgctttatgtattgcaacttaacaaacaaacaagtcaaattcttttaagaagttctgctgctttttcaattggattaaaaaaatgtcaaactgctacttcaaaattaattaatacaattatctgcatattcactcatatacatgcctgttaatattttttagttgttaatatttgttagtgaaagagttgatcagttgttaatatctgttagtttgttgaagcatcttgtcatttgaagggaaagctatctggtctctcaaatatccagggaaatcgttcattttgtatgttctacattaacctaagtttgccacctcatcaaaaaataatttgatttgttacattgttcgcgtaattgattgcaaccctttcatatatactagtaacccaaaactcattaaaactgggcttctcaaaagtcaatcatgtttccttcaaacttaacatatttgcctttgtttcaactctctaatgtaactatggagcagtaatataatcaattttgtgggaaacaaaagcaagagatcacaagtatcaatgaccagtggcaatctctcaaagagcacagaagcaacaaggttgtcggagcttgtgctgaatcctcgcctggtaaagagaggccgaaccagtgacattttcatcttgaatctatgcatatgaaaaatcatcaggcacttctcaaatggattacattggtatacaatatgaatctatgcatatgaataaaaacaaaaggaagtacccaatgaatgcaacagttaaatcgtcccaaactcaccataattttaaaagctttgcaatgcaaaatgaatatgctttcatggattacattgaattcaatggaacactgtttcacttctcaaatggacttgccggtttcctttggatcagaatctatcaaatgggatattggatgtgatcttacaaattatttagaggtagagggagaaaacatataaagcagagataaaacagaagcaaataacgagatgggttataggatgccgttttcatgtcacttaattttctttcattttaaactgggatcacgaaattgattccattcaataattctttgtattaaactatttagtattttagtaaatgaattcaatttaacaaataatatattttaaaatataaataaatattgaatgtaaaataatgtctcgctcacaatgtatatttgctattatttcaagagaaatttacaaatatacaaaattgaacatgaatttttcacaacaaaataatgttgttttaaatacaaagatcttatagcaatcatataagttattaaaattttaatgtatttaacattggtatacataattttgttgagataatataaaaatatttaattttaaataattttttaatagtgtgaaaataaaagaaacaaaaaagaaactaatttcctattatagttgagtgaaattcattaaacacaccactatagaaaagatatttaatatcaattttaaaaaatatattacaccgattccaagaaacatagtataagacaatgtaaaatataaaacaaattctatattggttattaaactgacttaaaaattttcatttttatattaatttttttataatccaatgcaaaaggggagtagaaactcactttctacattagtcaatatcataaccaatgccggataaaaaaaagccataaacaatgaagaaaatggcatgtatgcaaaacatgatcattttctatatcagatatgaaaataacaatgtagactgcgatttttctaatcggttattgttaaaagtgatgttaatgtatttttttatttataaacatattgtctattttatgttgatcccaacatgtaaatttataatatttaaaaatatagcctattaatgtagaatgtaactttttaagatttatgcttttcatctcttgcgatctacattatgacagatcaaagttcatgaactttttttcattactatttttagtctcaaatgtcaaagcattaattaataatgtagtaaaaatattagtagtctaatgatttatcacactatcaaagtgtatgacaagtcaatcacatatatatatatatttttttgcaatccgtattttaattttttttatttgtccaaataattaatataacatttcaaactttcacttaattaggaaattaaatattgatatttcgccttgtgagtagaaggcttaaatccacattgagctcctctaagagggtattaggatttaggagccaaatgaatggattgaaatttttatccaaaataatatggtaatgtttggtgtatgataaaaaaaagagtagacactttctctttagacaccatttttttgtcgtaccccttcattcatcttttttatgtcttaattagaaatatattttaaagaagaataaataattatttttgtttatgaatgtgtaaaatattgataattttatttttaaaaagatgaaaaatttaattttagttcctgaaaataaaataaaaacacgataaatatatttattcgttaatcttcatccattgttgttaatgaaaaagtttatgtgatacattccaaaataaatttattattattttacacatacaaagacaaattatattatctccttctaattcaataaaattataaataatagatataattgtataaagaaagtaaaatttacaaccgtacatccacacggggtgccgtactagtttaataatgctacgataaggtaacaataattgaggtgggtgacaacgtgtttcatatttgagttgggtagttggcaacgcgtttcataaattagttgggtaggtggcaccatccaattcgaagaaccaaaggtttgttcgtttatttatgacttatatcggtcatttttaatttgtttaaaatgataagaattttcactcctacaatgctgggtgtggttcgaatgagtataaaatacctatacattaacatcttaacattttggaatgaaatataatattaaacttacttcgtgacctatttaatagatgttttttatgtttaattccctcaattacatatcaggtataacaaagtgcatttgtagatcctatttaaaatacatatatcatactttaagccacaatttgtaagctccttaacaatttttttacagaacacatgcatacttcattatgcaagtaaattttcttttcttctacctattttataagatttttttgtcgtcaataaaatgatattaataattaattaaaataatttccttttaaatatataaattaaatatgtttatctaaaaactaattaaattatttttaaatcccatttttttatttaatctctatacattttataaaaatgttatttgtagttcatcggaatatatagataaattttgtcagcaaaatattcagataatcacttattaatgatgcattgaagataagatgacaattgatatagtagggaataaaccttcgtttatacagagctcgtttagataaaaaaaaattcatagaaattttaaggaaaaataagaaaaaaaacttatccataaacaaaaattactatttaaccttttaattacggcatttaaaaaatcatgttttaaagaaaataatgagataactcttataaatttacttgtgcataaataaattgtatcttatgtagttcatttgatttttttcttatttcttttattaaaaatatttataaaaaagattatttaaacatgtcaaaattaaatttaagatttaaatatatttttaatttctgtaagtttttattttttttgattttttattcttgtatgatgttttgtttattataaatttttataaatttaagtcttttaattttaattccattaattttggtctccataattttatgtttatatagactgaaattgaaaaaaaaatatacttataaaaattaggaagaggggactactttttgttttttagttactccagcgtagctgttattgcttgactttgacagacgaccaaactgggaagaaattatttgttgaggcaagtgatttgatatgcatgagtatgacagagaccttaatgttttgcctgatttattcactgtttcaaagccaagtaaatgcaaaggggttcagtcacccacctcactaaccttgaatttacttcagtcacccactaccaccttagaagccacactttacattccaaacagtaatgcttatattttgtcatttgtttggtcatgctctcagtattatctttgcttctttactccttgttggttttctacggaatggtagtacgccatccatatccatatccttcaatcatgtttagcttgcttcttccttaattaatctgctcttttctcactttcagttcattgggatagtgacaagtaattaaagacaagtcacaactcactagtcatccaattccaagaagacaggtttcttcctttcttaatttatagcttatattggctctttttttttcatttatttaagatttgaagaaatttcactcataaagatggatatataaagtgcacggtcaaatctttcaactctcgtgactaatgttatcaaaaagtaatatattataatttaagccacactttaatttgtagctctttaacaaaactttgacaaaatgcatttcattcaaacgtccaataataagctttacaatgcagcagcttcaaaacgagggaagtattcataaaggagaatacagagaggcaaatataaagaagaaaagaaatgggaaaatagagtactttggtgaagaacaggagatttcctcattcttagtttgtctcacaacaaaggtaagatccttaatatccttatcggttaaatgcgcagtgtcgccccgtgttttaatttatctattgttgaaatgaatgcttgtaaataaatatatttgcatgataccaatatgtttgttcctagcattttttttaattatcaatttaatacccaaaatatattaaaagatttaatttgatttccattttaaaaaaaaaattaatttaatttacaaattatttaaaaacagacccatataatctttttcgtccaattaagttatcgttagaataacaaattcaaacaatttttgtatacaaatagtgacaactactatatttattattagttttaaaaaataaaatgattttcattcttttttttcttcttcttcttcttcactttcggttctattaatcctcaaaagggtttcacaaagtgctgtttaggtggattagatcttggcttgtgggtgggttaaggtggtgatgatggagttggagcagtgacgttgggggcccgagtggtggcgtagcggtgtagtggttgtgcagtgttactatttgggtgggttagatatggacttgtggatgttgtttgggtgacttaagattctgatgatggagttggtgttgcaggtaaagggtggcatggcattgtggttgtggtggccaatgtgaagaagaagaaacgataatcgttttatttttaaaaataataataaatataatggtggttaaaaattatcattgttcataatcgaaagccgctgaaatgtgttatcttaacgacatcaactcaattggaccaaaataatcaaattgatcagttttaaaataatttaggattaaattaaacatcttaaaaatttggaaatcaaattaaatcttttaacataatttggtaataattaaactttctttttttaaggatattcctagcttttgaattagtagtgatgtatattttcattatttaactatatgctttattaacaaaagcaattgcctcccaagttacgtcttgttagaattcatcattttgtcggtgttctggggaacaaaagcaaaattctttgctacggaccaaaatatacattcaaacttagtatctttatttatgtttattcaaatttaacattcatttataattttaaacacatggtttcttgatatggttttagtaaatgcactttgatcgagttattgtgccaaatctgggaggaggtgtctcgtgagcgttgtgtttgaccaaccagtcaacacaactgttcccttctctaagagtatagttggaaaaaaaaatacttaagcctgacttatccattttgagcgaccttgtgttacaattgtattgtgatcatcaacatatatgttagaaatcatttggataaattatttttattatttaaataaattattttttagtgtcttggttttgtaattgtattatgatcatcaaaatatatttaaaaataatcttgaatgaacggattattgaattgaatacaccttgagtccaaagaataagaaactcttcaaagctctaacaatgatgatcaaccaatgatcttggatgcatgatacttaaagcatgttaatctctcatgaaaaatatgcataacctcttgttgactctttacagaagtgaaagataacattcgtaaaagagttacacaagaaaaagaataagagtgagtatttatatatttaactaaaaaaatagatttaacatattatcaaaatttgcaatagattatttcaatcaaaataaccttgttctacattttcaaaaacaggttattgatcacaaattaataaccaattatctcaatcgacaaagagcaatcttaacttagaaacaatataatcaataatgataattgattatattgatacacatagtttccttttttcttaaaacacacattttaattgattacataatgtgataattgattatccaatgggaccgaactaaatttcaacaaacaaaagtcttcgaacttgttctaacacattacatccgagacttatggatcaagtgcacgatcaaatctttcaactctcatgactaatgttataaaaaaaataatatattataatttaagacacactttaatttgtagctctttaacaaaactttgacaaaatgcatttcattcaaactacaaagcagcagcttcaaaacgagggaagtattcataaaggagaatacagagaggcaaatataaagaagaaaagaaatgggaaaatagagtacttgctgaagaacaggagctttcctcattcttagtttgtctcacaacaaaggtaagatccttaatatccttatcggttacttgcgtcccgtgttttaatttatcaaatgggacctaactaaatttcaacaaacaaaaatttttgaatttattctaacacattgtaataaattacatatacttgtaatcaattaccttatgcaaaacataacttacatcagagacttatggatcaagcaaattaagaaacataaatgattatccctaaatttgtagaagataagaaaactaatctaaagcacacaagcctagctaacacaaacaatataatgccacacatatggtaaatgtgttttgagtgtaaaatatcaaaatcaaaactaaaatggagataaaagaggcttcaatcttttttttttaatcataaccctttaattcatcatagaaaagacagacacacaaacaaacaaaatcatctaaattaacattttatcatcaaaattatatataaacagcacttatgttagcaatttaatttttttattaattgtgattagcttgtagcattaaattaaattacatcgaactactattacaaagttaaataatcaatctaatgattagaaagcttcaggtgaactttattatatatagagagagaggagagatactatttaacgttatatattattatttctcatgaaatatatattagagtaactaatgtagaaaatgaacataagtagtgatagaaaatttaacattactacttaaaattaaaatgatctaaataaacatttaattattataattgtatattatatagcgttttattacttaatatgaaaattatcaaaattatttttttgattatcaaattgtacactacctccgtaaaagaaataaagataatattaaataagattatggtaagcaacataattttgcattaaataaaatcgttaaaaatatgtataatcattgattattatgaagctgaggtttcgataatccacaaacttttattcaagtatctcttactatttttttaataaaaaagtaacatttaccataaactagattttaaatccatgcattgcaaaaatttctttaaaaaatatttttttatattttacttaatactttttaaaatatatttatcaataaattaatattttttaaaatatttatattaaatttgaggaagaagattatctatattcttagttaagttcatttataaatgttttaaatgtgtttattgtaaaaaaggtatatctttagaaatttttaatagtgtaattttattaattaagtatggttgttaattatgttaaaatatttttttttcaaaaaaaaatgtttgtaatttcggaaaaagaaaaacgaattggtttttttatatagaatcctcgtatggataatacatacattttttctattgttacatttgaaatgttagattagtgttccaatcatatggtatattgcctgaaacgtaggtttagtggttctagaatcatttgcaattgtaacccctccaaaacttcccataattcagccacaaaggttgttcctctaccaatcctctttgaaaaaccagaaatccaattgccatcagtgtcttgaatcaacccccgacaaaggctctgaattttttatcagtttcacagaacattcaagcatatcacacagatatggagagaaaggggtgaatttctgcatctctttagtctaataataagattcttattattccttaatagattaattatgatatctgactatttaaacacataaaatataaaataaagttcattaataataaataatataattatcttaaaatattagtgtaccttaattattactgaaacctattacaacaataaaaaaaaggttatacatgcagaattggtacacaggtaatgagtttaaaaattcgtagatgtgcaccaataggatgaaatgtgtttctgagtgtgacgctaagcattcttctttcttctgagaataaaagaaaaattctttgctaaagacaaaagtatacaatatacattcaaatttgatatcttttttttttttttgatttttaactcatggttgcttgatttggttttagtaaatgtactttgattttttattgatcaactatatgatattctttcctcattagcttttgactgatctggttccactcttcattttgttgaagagatgtaattgtacctagtacaagaaaatcaaggtcaaagcatggacgctgtgtcatccgcactactagagccagtaactaattctttgttggatctgcttaaaaagcaactggattacatccattacagtcgaaactttgatgaactacgcgagtgtgtaaagcagcttaaacttgtaaaggagaaagtagatcatcaatgtgaggaagctttcaaaaatggacacgaaattgaaggtaaggctagagaatggttagggaaagtgggtaaatttgagacagaagtggagaagtattggaacgatgatggccacaaaaagacacggttttccaactatttatttctttacttttgccatagactaggcagactagcaaagaagatggcagttgagggtaaaaagataaccgatgattgccccaagtctgatgaaattgcccatcgggtatacgtaacatctaatgatgccattttgtctaataatgaccttatggattttggttctagaaaatccataatggaacaaataatggcaacacttgttgaagatcccactgtgaaaatgattggagtgtatgggcgaagtggggtgggtaagagcactctaatcaaagcaattgctgaaattgctcgagacaagaagttgtttaatgtggtggctttttcagaaataacagacaaccccaatctaaaacaagtccaggaagatattgcttaccctttgggattgacattggaaggagaaagtgagaatgtaagagctgatcatctacgaaggaggttaaagaaagagaaggaaaacacccttataatcttggatgacctttgggacagattagacttgaataggttggggattccacttgatggtgatgttgatgataatgatttaagcaagaagacaaatagtgataaacagggtcccaaagggccgacaaaagaaaaatctcttggtgattataagggttgcaaaattttgctaacttcaaggaaacaaaatgtattaacggataaaatggaagttaaattaactttctgtgtagaggaattagatgaaaaagatgctctgaagttgtttcggaaggaggctggaatacatggtgaaatgtccaagtctaaacaagaaattgttaagaagtactgttcagggttacctatggcaataattacagttggaagggcattaagagacaagagcgactcagagtgggaaaaacttaaaaatcaagacctggtgggagatcagaatcctatggagatttctgtaaaaatgagttatgaccatctagaaaatgaggagctcaagtccattttctttctttgtgctcaaatgggtcatcaacccctaattatggacttggtgaagtattgctttggtttgggaatacttgaaggggtctactcacttggggaagctcgaggcaaaatatctacatcaatccaaaagttgaaaaactcaggtttagtgttggatggaagttctagtattcatttcaatatgcatgatctggttcgagatgcagctttatctatagcacagaaggagcacaatgcatttactttgagaaatgggaaacttgatgattggcctgaactcgagaggtgtacttctatttttatatgcaacagtgttatcattgatgagcttccagaagaaataaattgtcctcaacttaaatttttccaaattgacagtgatgattcatctttaaaaatacccaacagtttttttaaaggaatgaaaaagctcaaagtattaatgttgactggtattcaactctcaagcttaccatcttcaattgaaagcctatcagacctcagattgctttgtttggagagatgcactttagatgacaacttatccatcatagggaagctgaaaaaattaagaattctcagcttttctggatctcgaattgaaaatttgccagctgagttaaagaacttggataaactacaattactagacatcagcaattgttcagtagtcaagaggattccgcctcagcttatgtcaaggttgacttcgttggaagagttgtatgtaagaaagtgcttcatggaagtgtcggaggaaggagagagaaaccaatgtcaaatttcatttatttctgaactaaagcatttgcatcaattgcaagtggtggacttaagcattccatgtgctgaagtttttcccaaggaattgttctttgacaacttaagtgattacaagattgagattgggaacttcgaaatgctttcagctggagatttcagaatgcctaataagtatgaaaatttcaaatctttggcattggagctgaaggatgacactgacaatattcactctcagaaaggaataaagttgttgtttaaaagagttgaaaatttgttgttgggagagctgaatggtgttcaagatgttattaatgagttgaatttggatggatttccacaactgaaacacttatccatcgtaaacaaccctagcatcaaatatatcatcaactcaaaggatttgttttatcctcaggatgtttttcccaagttggaatctctatgcctccacgaactaaataagatagagatgatatactttagttcaggtacagagatgatatgctttagtccatttacagattgctcattcaccaaattgaaaaccatcaaggtcgagaagtgtgaccaattgaagaatcttttctccttttgcatggttaaattgcttgcgagtcttgaaacaattggtgtttccaattgtggttctttagaggagatcattaaaatacctgacaattctgataagattgagtttcttaagttgaagtctttgtcacttcaatcattatcatcattcactagtttttataccatagaggggtcttctacagacagggatgagatacaaattactgttgcagagaatgagcatagtgagatggctcctcctctttttggggaactggtatgatggtgcatatttttcctttaaaaattttaattagtctgtgactacttatttttcagttgcaaatatgttaattgtgtatgtgcttgcaggttgaaataccaaacttagagaacttgaatttaatctcaatgaacaagatccagaagatatggagcgaccagcccccgtcaaacttctgctttcaaaacttaataaaattagatgtgaatggttgtcataatttgagatatctgtgttcattgtccgtggccagcagcttgaggaaactgaaaggcctctttgtaagagaatgtcaaatgatggagaagatttttagcacagaaggaaatagtgcagacagggtgtgtataggtgtaaataagtaaatttgtatagttgactgaatgttatgcatttctgtttagtttcgttaccaaataatggtttgttttcatcaaagtgcaggtctgcgtctttcctaagttggaggaaatccacctcaaagaaatggataatttaacagacatatggcaagctgaacttagtgctgattccttttctagtctcacttctgtgaacattaaaagttgcaataaactagacaaaatttttccgagtcacatggaaggatggtttgcgagtttgaacagcttgaaggtttattcttgtcggtcagtgaaagtgatttttgaaatcaaagattctcagcaagcagatgcatctggtgggatagacacaaatttgcaggttgttgatgtaagggtactcccaaagttggagcaggtgtggagcagggatccaggaggaattcttaacttcaaaaaactgcagagtatagagatgtttggttgtggaagactgaggaatgtatttccagcttctgtggccaaagatgttccaaagcttgaatacatgtcggtctcagaatgtcatggaattgtggaaattgttgcctgtgaagatggatccgaaacaaacactgaacaattagtgtttcctgaactaacctacatgaatttaagtggcctatcaagcatgcagcatttctacagagggagacatcctatagagtgtccaaaattgaagaagatgaaaatatacaattgtagcaagaagctaaaaacattcggaaccggagaaaggagcaatgaagaagatgaagcagttatgtcagctgaaaaggtaagtcatatagtgagacataaatggagagtgatattgtaagatgtgtggcatactttaaaaagatatagccctgctaaatataatatggattattaatttaacgtctgtggtcccctaatccctcttttatttagaattgaagggaatgtttgattaattattataatttaagagcattttttagtcttgatgggaccccagagtaaccaatgtgctccatcagggataagcttccttcttcatagtttactatttacttttcttttatttttctgtagatattccccaacttggagtgtttgtatattgactttgacgaagcacagaagtggttattgagcaacactgtgaagcatccaatgcaccgtttaaaagagcttcacttgcgcaaagttaatgatggtgaacgtctctgtcagattctgtacagaatgccaaatctagaaaagttatacttgtggtacgctgaacatttgcttaaagagtcgtcggagtcacgtttgggaaccgtattacagctgaaggaattggttttgtcgcagtcaaagataaaggatataggatttgaacgagaaccagttctacagagactagagcttttgagcttaaatgggtgtgacaaattgaggaatttgggtcctccctcggtatcattggcttacttgacaaatttggaagtatggtattgtaaaggattaaggaatttaatggcatcctcaacggcaaaaagcttggttcaacttaagtccatgaagataagaggatgtgatgaattagaggaaatagtaagcaatgagggaaatgaagaagcagagcaaatagtgtttggcaaattgattactatagaacttgaggggctaaaaaagctgaaaagcttttgcagttacaagaagtgtgaattcaaattcccgtcattggaagtattgattgtgagagaatgccgaatgatggaaagattcacggagggtggcgcaagagcaccaaagttagaaaacatagttagtgctaaagaagaaggaaaagaggaagccaaacggcagtgggaaggagacttgaatgccaccatacaaaacaaggtacttattcatttatttatgatttaaatatgtttttgtccatagtagtaggtatatgttgaatttgtccatatgatttaaatatgccactcttaaattttacctttatatacttgagattttcaattttaatttttatcttaattgatctttaaatttcaatccgtttgcaaatagataccgtgaattttaattttgtttcatttttttgttaaattaaattattttctattgatcttaaattttattatttttacaaataaatatcttacacctaaatttctttttaattgatccttaaaatttatccgcaaataagtatcttaaaccttaatttcattctaattagtcattaaaacttatccattttacaaacagtagttctttctatcaagttagagttaaattatttatcataaatgccatacattcttaatcttttaatttgttacaattcatttaatttttttaaaaaataaaactaataaatgattgtgatttaaaataaatgaataagtaaatagttacgatttcttctagtctaactcaatttaagataaaaaaaaaagactattcaaaaaatatatagataaatttaagtgattggacttaaatttatttttaatccttacaatttataattttttgttttttacccttgtaaaattattttttatttttattctttataaattatgtttgttttattttttgttcttaacactttaaatgatacttttttatttttaaaattctattaaaagaacattaaaaattcaatttgcaaagattaaaaattgaagaatatgacttagatatgttttggcttttgcaaattagagttcctagtaagtttatttttctaattttagtgtatctaagtttgtgttttttaatttgatctagataaaatattttacttatctttaatccatttaagtttgtgttttttcaattttaattcctataagtgagaaaatcttaatttgtaaatttttagtcattcttgaaaaaccataaaaggccaatttttaatcattttcattattattcttatcattataaacaattttatattaatattgtattaaacttttattttgataacattattacttacggctaatttgataacacttttatacaataactattttattaattaatcgtctttttataatatagaactattttttgcatttcttaaacccgtttcaaatttatatcctcattctggtattggaaaacttgtagaatatgacttgaatttgaaagtttttcttgtagtcttttattcagatttttctatgaattttgtgttttgtattttgtgatttttttctacaaattgctacaaattttttccgtttaattttttcttgcataatatagtaactgatttatgactaatctatctatttgttataatgtaatcgccagctatgaaactgacatattagccttttgtatataatgtcatattttgcatgcacagattttggagtctgcacgtactgataaaaggctcacagtcagcccactactacaagagatatggcttggctcacggccgatcccaaagtcgtgcttcagtaacttgaagtctttgactgtggacggatgccaacttttaacagatgttgtcatacccttctatttacttcctctcttaactaatttgcaagaattacatgtccgaaagtgtggttctgtgaaaagcatatttgacgtgaaaacagctatgggattgggagcagcagccttccctagacctctccctttttccctcaagaaattgactttagagaggctgccaaaactggagaatgtctggaatgaagatcctcatggaattctaaccatgcaacttctacaacatgttattgttgagaaatgtaaatgccttacaagtgtgtttcccgcatcagtagccaaagatcttgaaatactagttgtcaaagactgtgaggaattgatggaaattgttgcagaggataatgcagatccaagagaagacaatctagagcttacgttcccttgtccctgtgtgaggtcattgaaactacaaggtttgcccaagttcaagtatttttattactgctcactgcagtgtgacatgttccagacacctaccgaagatgaaatggtactattttcttactgcagaactgatgcctgtaaattaaatataattgcactacttttataagaattgaataaccaccattagtttctatacatcatctttattgttcaatgagtgatatgaacatggtcttaaaattaaaatgtgagaatttcacgtacattactatatatgctgtgttctatcttctatgcctattaaccctcatttttcatatgttttacataaataagtaaaagaatacacttaagggtaaatgttgcattcatgatacaacaatgacattttaggtttgagctttattcttattaatacagacaattacagagaatcttacacacgccattttactttatcaaatatcatgtatggatggttctcaaatctttaaaaacaatctttataactgctctaacctgcttttgcttggttatcggtttctgttcgtagcctacatccaacttgaagtgcctgtcactcggtgaaaaaggactggagatgatcaagcgtggagaatttcagagaaacttcatacacaagttacaagttcttactctgtgctttcataatgggtcggatgtatttccatatgaaattctacaactggcgcccaatatagagaagcttgtggtgtacaatgcttccttcaaggagattaatgtggattacactggactcctattacagctcaaagacctatgcttggagtcccttccagagcttgtttccattgggttagagaactcttccattcagcccttattgggaaatctagaaaccttggaagtaataggttgttctagtttaaaagacttggtaccatctacagtgtctttctccaatctgacatatttggaagtagaaagatgccattgcctgctatatttgttcacatcctccacagcaagaagtttgggtcaactcaaaagaatggagataaaatggtgtgattcaattgaagaggtagtagtctctaaggagagggatgaatcacatgagaatgaaataatatttccgcagctcaaatgtttgaaacttgaaggattacgaaagctgagaaggttctacagaggaagtttattaagtttcccatcattggaggaattgtcagtaatcgattgcaggtggatggaaacattatgtccaggtaccctaaaagcagacaagttggttcaagttcaacttgagaagagttatcctaggagtagtcactcagatgctatcaaattggaaaatgacctgaactctaccatgcgggaggcatttaggaaaaaggtacgttttcaattattttgattaaatatgattgatattggtgtatgttgttcttataatgcaaaatatacagcgttaggttaattagattcatcttcaaattaactttggcctcatccaatttatcttggattctcagcgtcgtccaatttttcggaaaaccaaaaaattcacggaaacaacatttgtgtgccaatcaaatttggctcatattaattatttacgttaaactcataatatttaaataatttaatttattttcttatgtactttatcaatgctacttaacttttatatatatataaattaataacactgatataaaatgcatccaaactcttttacctttgcagttatggaagtctgcagattgggaatcagttcttgacctcaaagatagcccactacaagagatatggcttaggcttcactcactgcatatccccccacacttctgcttcacttacttacacaccttgattgtggacggctgccattttttatcagatgcggtcttacccttctctttacttcctttattgcctaatttggaaacattggcagctcgaaactgtgatcgtgtgaaaatcatatttgatgtgacaactatgggacctctcccttttgccctcaagacattgtatttagagcggctgccaaatctggagaatgtttggaattcaaatgttgagcttacgttcccccaagtggagtcattggcactgtgtgatctgccaaagttaaagtatgacatgttgaagccatttacacatctaaatcaactctgtattcaaaaggtatcattactacttctatatatatgaacaaattgccatcctgttaaaattaaaatgttgtgaacctttcccttatacagttatatatgctatgttgagtcttctatccatcttctacgcatattagcttccatttttttatgatttatctacattaagtcagataatacagttactattcaatgttgatcatgatgaatttatgatggaacatgttgcaatattcatgatgtgaaaaagcatttgaggtatgatccttattctcgttattttcttctataaccttcctaatgacttgtttgctcctattgaattgacttggttaatttgtcattaattattagacttattttagcatgaaagttcattatttcaattatataaaaaagagctggatctataactgccctaacctgctttttcttcattgttggtttctcttcgtagcttacacccaacatagagcacctgacacttggtcaacatgaactcaacatgattttgagtggagaattccagggaaaccacttaaacaagttaaaagttcttgccctgttctttcatattgaaaccgatgtatttctacaacgggtgcccaatatagagaagcttgaggtgtgtgatggttccttcaaagagattttctgctttgatagtcataatgtggatgaggatggattggtttcacagctgaaagtgatatgctcggactcccttccagagcttgtttccattgggtcagagaactctgggattgtgccctttctcagaaatctagaaacattgcaagtaatcagctgtttgagttcaataaatctggtaccatgcacagtgtctttctccaatctgacatatttgcaagtacaaaattgcaagagtctgctatatttgttcacatcctcaacagcaagaagtttgggtcaactcaaaacaatggagataggttggtgtgattcaattgaagagatagtgtcttcaacagaggaaggggatgaatcagatgagaatgagataatatttcagcagctcaattgtttggtacttcaagaattaggaaagctgagaaggttctacaaagggagtttaagtttcccgtccttggaggaattcacagtaattcattgcgagaggatggaaagtttgtgtgcaggtacagtcaaaacagacaagctgttagaagtgaaattcaggaggttaggaggtgttatccgattggaaactgatctgaactctgccatgcaaaaccgataggcatttctggccagggtatgtctttattttttttattcaacgttattataattgttgttataatacaccgtgtcagtgatatgttacgtggcacgattaacttaaattttgagaagagtttaattgccgtgtttaatttataattaaattcagaaattttttatctttcgccgtctctgtcatagtcgtgtcattattattattattattattattattattattattattattaactaacataatttttataaattgtgtaggcaatgactccaatattagagttagaggagatggattcaataaaaagaaaatggttagaggagatcaacaaattaagtggcccggtgcctaccgaaaaaaaaacaaagtggcccagttttattatatttgtttgtttgtgaataaaaaacgaattgtgcgtgtggtctacatagttagtggtgaaatacactgttctattctatttattatgtaatgtttgtgattataagtcttaaacgaccctggagaataaaaatgaagtgatgaactgtaagtttgtaacactaaaacttgactctattgttttatgacaaaattaaataaaaatgtgtcctaaattatgtatgttcaccattcattttacttatatttcaagattcaaagtcttcaacataattcttgaaaaaatgtcttcaacataactttgattgtggtaggagtttaattatcactttgtccatctcctacgattagcatctaaagactgtgttagctttaatttagtaaaggctaacaacgggattgattatatctttgttgtactaaattgttataatgaatagaaaaatttcataatttattgaaatgctattttttaattttcatgcggtgttaatatagttttcaagaaattgtaacattactaaaaaaataatttcaaatatcatttttaagtattttttatgatgattctcaaccatctctaaatctgtgttgtgaaaatacttttcaccgtgattttttaaattctataagaatattgatttttacattgatttttttgaaattatttagattgtattatttattatttaaaaaatgttaaaaatattaggattctaagatgattttaataaaaaattaagatgatttttagattccaatgttgtttattattttaatacaaaatttcattaaaataataaaatataattaaatatgattaaaaactaatatttatagcaaataaatcacaacgtaaactatggaccacaaattatatatttacggatccctaatgctagattcctttctaaaaaaatattaaacatctatcaatttgttctaaatcaaatctaaagtaatttaacactgtaggtttaagatattccaatcaaacaatagaatacaactttacatcttaaaagtgatataattttagtattttcattaaaaaattaaaggagacgtctttgcatgttaaaatataaaagttaaaaatgaataatttttttaaataaaataaaattagtaatatttaaaggatgataaaatatttaattcaaccaaactgtagtagtcataactaaaattttcagatttgaaattactaagattaaaccataatacttctttgtcatttaatctgtgtgcattaaggtatcatattgtcttatttcataaataaagaaaaatatggaaagaaaaaactaattaatataaattagtcataactaaaaaagtgaagaaaaattaggatatatgctatgatagtatttacacgtgctaattttaattaatttattttttatggtaattaacttttgatcaaaataatggaaaatattttttaatcaaaattgaaaaattaattaaaagttacaaaattaatttttttacatgatattgttaactgatttattaaattagaaatatttaattggtaaattaactgttaagattactagtaaagacataataatataaaagatatatttatgatttatataaaattttattgactttgtgccgcaatgatgacagcacgtccccatctctagagaatggataagccatcctggacaaattttaagcataacattatattcatacgtaactaattagagaaattaaatccctctcatatggaaaatgcactacgggaaggtaatattattattattactaattgaaaaatagatattttgtgtcttatgtcttaattgtttttttagttagatgtaggtgtaataattatccttttaaaaagttattaaataataattacttataatttgggggtgttttttaaagtaatacaagaataaattatgaataataacaaatgtatataaaaataactacctttattaataattataaatttaccatgaattgctaaggaccgaactcaaaccacagaaacagggacaactaccaagcaaataatgcccccaattccatggccgtatcttctctgtaaaggcagataaaacatttcagcaatggcatacccaccaactataatagaaaaaactagttgtcaaagactgtgagggattgatggaaattgttgcagaggataatgcagttccaagaggagcaaatctacagcttacgttcccgtgtccctgtgtgaggtcatcgaaactacaaggtttgcccaagttcaagtatttttattattgctcactgcagtgtgacatgttccagacacctaccgaggatgaaatggtactattttcttactgcagaactgatgcctgtaaattaaatataattgcactacttttataagaattgaattaccaccattggtttctatacttcatttttattgttcaacatgaatgatatgaacatggtcttaaaattaaaatgtgagaatttcacgtacattactatatatgctgtgttctatcttctatgcctattaaccctcatttttcatatgttttacataaataagtaaaagaatacacttaagggtgttagttttttcataattccgcaagtatcccatgtcttcagtaacaaacataaaccatttgttatctaaatatttagttagaatactgagttgttgtcatgtgaaaaatctgtttctaatatggttcatcttttgttcaggttcaactctaatatttgaaaaagtgttataatcattatcccataattatctctcatatcctgttgcacacttgagtttgagtttgttgatgaccagaactctatataaagtgtgtgctaccgcggtcactgaatccgcccctcttttcatatactaacaccatcaagtacgagtgagtaaggaattggaatcacaatcaattacgaagttgccatggagcaaaattgccaccctcacaaacgaacgttcataacaacaatagcaatggtgggagattagtcatgtctcttttagcttccgaaataacatcaaaatttggaatgatataaattttctattttttgtgcgatttaaaatgtgtttttgggtgtctaaaatgtgcatataatggtgtgggttttcttaatcgggttcgtggatattaattggatctttatgacccgggaggttgaagatgcacatgcgcgcataaatgaaaatgcattaggctggtctcgaacccttgccctgcaaggtgaaaaaactccctttgccactagactattgtaatttatttgtttattagatgcagttcatgtttatttataacttctaaaggataaatcatttatgtgcaagctatttaaaattgtatgtctctaagttatgttgaacatgaaatactatgttaaatactagtatgcattaaatttgatcctttaaagtttattacatgttgaatggatatacatacaatattattttgaattggtatacatgtaatttttatgatttaatattgagcacgtttatattattaagtatgttatgcaaagattatttttgaatgttaagtgattaatataattttattaaattagagagtagccacaacatctttaattgagtaaaattatatgttaaatttataatcacattagaaatattaattgtgattaatcatatgtaatgtgataaaatctacccacaggaattttgttatttttgtatgattaattagaataaaatattagattatatttcttaatttacccacaagaattaaggaaaagaacatgatttaatagttttatcataaagttgcatgatgaatctaattgggtaggactttagcccacaggcaagttttgtgtcactagattcatatattgagacatgatttgcattggcaaaacatgacatttgtttagtctcaaatttgcttaatgagcatgtgtgtttgtttgcagttttacaatctatgaatatttcttttgaccttcccatattaaaaggtgataattataaggtttcgaaggaaagaattcttcttcatttgggctggatggatattgtctatgctataaggaaggatgagctaccggctattactgaaactagcgaacctgatgttgttgacctttatgaaaagtgggagaaatataatcgtctctccgttatgttcataaaaaccaacatattcgctagtatccggggttcagttgaccagcatgataaggttaaagatctgttgaaagccattgatgaacagtttatgacctctgagaagtcgcttactagcacactcattatgcaattctcttccattaagcttactggaacgggaggtgtgcgtgaacatatcatgcgcttaagggacatagtggctcagttgaaaaccctggaagttaccatgtctgaatccttcctggtacatttcattttgtgcaccttacctcaacaatatacacactttaaaatctcctacaacacacataaggataaatggtctattaatgaattgatgaccatgtgtgttcaagaagatgagagattgataatggaagagggtgagaaggtaaatttgactacttctacttttggaaaggataggaagaagtttgtaggcaccaataaaggaaggattccgactcaaccaacaattaaaaaggagtcaaagtgtttcttctgtaaaaagaaaggacacataaagaaggactgccccaaatttaaaagtgggtttgagaagaaaggtacaccatttactttcatttgttatgaatctaatatgattaatgtgaatcataatacatagtggattgaccctggttctataatccatgtttctaatatcttgcagggtatggaaagcctaagaaagctaatgggaagtgagcagtgcatctactcagggaataggatgagctcgcatgtagaggacattgaaacgtgcgtcttagttttaagtagtggctttaaattacatttggagaaagttttttatgttcctagtttttgtaaaaacttaattttttttttaaacttgcacctttgggattctattttaattttacagactttggttttaatttactgaataaatttgaaattattggttgtggtcaattgattgatgttctttattcaattgaattgaaaaacgacgctacttataattctatgcacgtttctgttgggttaaaacgatgtattgtgaatgaagaatcctctatgttgtagcaccggagattaagtcatatctctattgagagaattaagcgattagtaaatgaatgagtacttaatactttggattttgctgattttgagacttgtgtagattgcattaagagtaagcaaactaacaagtctaaaaagggtgcaaataggagttctaattttttagaaatcatacatacagacatatgttgtcaagacatggatgcaaataattcgaaatacttcataacctttatagatgattattcattatatatgtatctctactaactttattctaagaatgaagctttagatgcctttaaagtttttaaggctgaagttaagaaacaatgtggaaaacaaattaagatcatgagatcaaataaaggtagggagtactatggtagatacacagaggatggacaaccaccaggtccatttgcaaaatttcttcaagaacatgagattgtttcccaatacactatgcgtggttctccgaatcagaatggtgtggcagaacgaagaaatcaaaccttattagacatggtgagaagcatgaggagtaatgtaaagcttcctcaatttttatggattgatgcacttaagataactgcgtatatattaaaccgagttccaaccaaggttgtctcaaagacaccttttgagttattcaagggttagaaaccaagtttgcgacatatacgcatttgaggatgcccgtctgaagtaagaatttataatccacaagaaaagaaactagaccctaggactattactgggtatttcattggatattctgaaaagtctaacgggtataggttctattgtcaatcccacaacactaggattgtagaatcaaggaatgcaaaatttcttgaaaatgacttgatcagtgggagtgatcaatttcagaacatttcttctgaaagggatcactatgaagctgaaccttctgggacaagtaataggttggtagttattcccacccctcaagttaaaatgggggttagacaaccagtgattgaagttccacaagttgttgaaagtgatcatgtagatcaagttgtttatgaggaacaacatgatgatattgaacaaactggtgaagaactggttgaacaagtttctcagcaagataaccaagcagcattaagaagatctactagagtaaaaaagacagaaattcctagtgattacgtagtgtacctacaagaatcagactacaacattggagccaaaaatgatcccgagacattttcacaagccatgagttctaagaaatcaaatttatggtgtaatgttatgagggatgagatggattctatggcatctaaccaattttgggatctcgttgagttgcctgttggtgtaaaacccatcggatgtagatgggtcttcaaaacaaagaaagactcagaaggaaacattgagagacataaggcaagacttgttgttaaagggttcactcaaagagaaggaatcgattacacatagaccttttcccctatatctaagaaagactttattcgagtaattttggcattagtaactcattttgatcttgagttgcatcaaatagatgtgaaaatgacgttccagaatggtgatctagaagaagaggtttacatgaaacaacctgagggattcttatctagtgttggtgagcacttagtctgcaagcttaataagtctatctatggattgaaacaagcctcccgccagtggtatttaaaatttcatgaggtcatttcttcattcagctttgaagagaatgtcatggatcactgtatatatcaaaaggtcagtgggagtaagatttgtttctttgtattatacgtagatgatattctgcttgcggctaatgataagggaatgctatatgaggtgaaacaatttctctcaaagaactttgatatgaaggatatggaagagacatcttatgtcataggcataaagatccatagagaaagatctcaaggcattttaggcttgtctcaagaaacttatatcaacaaagttttagagatatttaatatgaaaagttaccaagtgtagctcccattgagaaaggtgataaatttgatttaggccaatctcccaaaaatgattttgaacgggaacacatgaaaaatattccatatgcttcagcaattggaagccttatgtatgcttaggtttgcactagacctgatattgcattcactgttggagtcttggaaagatatcaaagtaccaggtattgaccactggaaagctgcaaagaaagtgatgagatatcttcaaggaacaaaggattacatgcttatgtatagatgaactgattgtctggaagtgattggctactccaattcagactttgttggttgcgttgataccagaaaatcaacatatggttatgtttttatgctagctggtggagctgtatcatggagagttcaaagcagtctttaattgctacttccactatgaaggctgagtttgtttcttgttttgaggctacctcacatggtgtatggttaaaaagtttcatatctgggcttagagttgtagactctatttccaggcctttaaagttgtactgtgacaattctgttgcggtatttattgctaagaacaataagagtggaagtcgaagtaaacacattgacattaaatatttagccgtaagaaaacgcgttaaagaaaggaaagttgtcattgaacacattagcacagacatgatgatcgttgatcccttaactaaaggcatgccaccaaagaattttaaggatcatgtagtgcgaatgggacttgattccataatgtgacattcattgtaagtacatttattgttttgatgaaattcttattaagtttgatgtttctcatttgttttgtgcacttatttattttgagaagtatcatttgaattttgacctaaaataagcatatggtttattcattaagttaagagcttgagaaacttgatatattgtgatgcatggaagataatacttgtttttagaggacttatcgtcatgattcatatattttgtttcttgttatgattagtgatgggttaaatggaccaagtgggagaatgttagttttttcacaattctgcaagtatcttatgtcttcagtaacaaacatatgtggcttgttatctaaatatttagttaggatactgagttgttgtcatatgaaaaatctgtttctaatgtggtccatattatgtccaggttcaattctaatatttgaagaattgttgtaaccattatcccataattatctctcatatcccgttgcacacttgagtttgagtttgttgatggcgagagctctatatataaagtgtgtgctgctgctatgatagatcactaaattcgttcctcttttcatatactaacaccatcaagtacgagtgagtaaggatttggaaccacaatcagttacgaagttgtcgtgaagcaaaaccgtcacccccttgaacgaatgttcataacaacaatatcaatggctggaggttagtcatgtctcttttagcttccgttgatcgtttagatccttttatggtaaattatcaaaggatacatgttgcattcatgatacaacaatgacattttaggtatgagctttattcttattaatacagacaattacagagaatcttacacacaccattttactgtatcaaatatcatgtatggatggttctcaaatctttataaacaatctccctaccatctaaaggatcgaatttttcatcattttcttctccgttggaaccactacaacctcccttaaggtctcctttatcactttttccatctaaaattttaaactacaaataaataaatttgtttcggcacatactgtatcaaaaaatcccttccaccttattaatagctagcctggttcatctcttttacaacctccctaacctgcttttgcttggctatcggtttctcttggtagcctacatccaacttacagtgcctgtcactcggtgaaaaaggactggagatgatcaagcgtggagaatttcatgagagaaacttcttacacaagttacaagttcttactctgggctttaatattgggtcgactgtatttccatatgaaattctacaactggcgcccaatatagagaagcttgtggtgtgtgatggttccttcaaggagattttctgctttgatagtcttaatgtggatgaggctggactcctattacagctcaaagtcttatgcttggactcccttccagagtttgtttccattgggttagagaactcttggattcagcccttactgggaaatctagaaaccttggaagtaatagattgttatagtttaaaatacttggtaccatgtacagtgtctttctccaatctgacatatttgcaagtacaagattgcaacagcctgctatatttgttcacatcctcaacagcaagaagtttgggtcaactcaaaagaatggagataaaaaggtgttattcaattgaagagatagtctctaaggagggggatgaatcacatgagaatgaaattatatttccgcagctcaattgtttgaaacttgaaggattacgaaagctgagaaggttctacagaggaagtttattaagtttcccatcattggaggaattgtcagtaatcgattgcaagtggatggaaacattatgtccaggtaccctaaaagcagacaagttggttcaagttcaacttgaggagtcatctgatgctatcaaattggaaaatgacctgaactctaccatgagggaggcatttgggaaaaaggtatgttttcaattattttcattaaatatgattgatattggtgtatatatatgttgttgttataatgcaccatctcagcatacttttctcttcatccctctcctgcaaaatatacaccgttaggttaattagattcatcttcaaatatactccaaaatctatcttggatagtcattttccttcaaagagattttctgctttgatagtcataatgtggatgaggatggattgctttcacagctgaaagtgatatgctcggactcccttccagagcttgtttccattgggtcagagaactctgggattgtgccctttctcaggaatctagaaacattgcaagtaatcagctgtttcagttcaataaatctggtaccatgcacagtgtctttctccaatctgacatatttgaaagtaaaaagttgcaagagtctgctatatttgttcaacagcaagaagtttgggtcaactcaaaacaatggagataagtgggtgtgattcaattgaagagatagtgtcttcaacagaggaaggggatgaatcagatgagaatgagataatatttcagcagctcaattgtttggaacttgaaggattaggaaagctgagaaggttctacaaagggagtttaagtttcccgtccttggagaaattgacagtaaacctttgcgagaggatggaaagtttgtgtgcaggtacagtcaaaacagacaagctgttacaagtgacattggaagggtatcttatcccattggaaactgatctgaactctgccatgcaaaaccgataggcatttctggcaagggtatgtctttttattcaacattattataattgttgttataatacaacgtgtcagtaatttaaatcttgaaaagagtttaattaccgtatttaatttataattaaattcagaaattttctatctttggtcggctttgtcattgtcgtcattattattattaactaacattttttatgaattgtttaggcaatgactccaatattggagttagaggagattgattcaccaaaaagaaaatggttagagaagagatcaacaaattaagttgcccagtgactaccgaaataaataaataaaacaaagtggcccagttttattatatttgtttgtgtgaataaaaaacgaattgtacgtgtggtctacccagttagtggtgaaatacattgttctattttatttattatgtaatgtgtgtgattataagtcataattaaacaaccatggagaataaaaatgaagtgatgaactgtaagtttataatactaaaacttgactgtattgttttatgactaaataaaataaaaatgtgtcgtaaattatgtatgttcaccattcattttacttatatttcaagattcaaagtcttcaacataactttgattgtagcaggagtttatcactttgtccatctcctatgtacaattaacatctaaagaatgttagctttagtaaaggctaagaacgggccggggttgattataactttgttgtattaaattgtgataatgaaccgaaaaatttcataatttgttgaaatgttattttttaattttcatgcgatgttaatatagtatgtcaagaaattataacactactaccaaaaatcatttcaaatattatttttaagtatttatgatgattctcaatcatctctaagtgttgtgaaaaataaacacttttcaccgtgattttttaaattgtacaagaatattgatttttacattgattttcttggaattatttagattgtattatttattatttaaaaaatattaaaaacattaggattttaagatgatttttttaaaaaaaaattatcttctaagatgatttttagattccaatgttgtttactattttaatacaaaatttcattaaaataacaaaatatataattataatatgattatgaactaatatttatagcaaataaatcacaacttaaattatggaccacaaattatatatttacacatccaatatgtgggatccctaatgccagattcctttctaaacaaatattaaacatctatcaatttgttctaaatcaaatctaaagtaatttaacacgctaggtttcacatattccaatcaaacaatagaatacaactttgcatcttaaaagtgatataattttagtcttttcattaaaaaaaaattaaagcacatgtctttgcatgttaaaatataaaagttaaaaatgaatcatttttttaaaataaaatagaattagtaatatttaaaggataaaaaawatttaattcaaccaaatcgtagtagtcataactaaatttttaggatttgaaattagtaagattaaatcataatagttgtttatcatttaatctgtgtgcgttgaggtatcatattgtcttatttcaaaaataaagaaaaacatggaaggaaaaaactaattaatataaattactcgtaactaaaaataaagtgaagaaaaattaggatatatgctatgatagtatttaaatgtgttaattttaatttatctattttctatagcaattaacttttgatcaaaataattgaaaaaaaattgatcaaaattgaaaaattaattaaaagttacaaaattaaaagttacaaggaaaggacaagatacagttgaatcgccagtgtccaattcctctatcctgtcatggaaccatatgtattaaaacactataagcattctttagaaagagtacaacatgctctcaactaggaaaggaataaagttgtatcaaaagtttcctcacttcaaaatcttcacacttcataagctgcgtgtttgagttctccagaagggatccaaaattagttcagttccttttgaatcgctgaagccctttaaatgagcctgcagggtccgcaaattcacaatcctcttcatacacatttaatgtcagatctcctgatgattgagaaaaacatactcaccacatgaaccgtacgtctgcaatttaaccaaatgaataactagaagatactaattggtattagtttgattttatgtcaattgtcacagcagaagcgagggcgaagtcccacgttagagcctgctctgataccatgttagatttcatcttaaaaccaattaacattaagtggcagatatataagctgcactcaaagaattgagatagccaatgtgggacttgcgtattttccaacagaagatactctgctacttgaagtacacttcttcaagtatgacccttttctgtgtagtaattcctatcctccacaccctctcccctaataagttgagtgtagctattatataattgtatacgtgacataagactagcagtgataatctctctgcctgtaccaagttgcatcaggggagctagcaacaatttgtagtacaagtcaccaaaccttgaagatgcatataaggattaaggaaatcaggattaaaaaagacaagaagccaaggttacttagaagggttcaaattttatcaggagggctttagtaaacactcgtaagtcatagcattgaagaacaatggtcgtaaaagaaaatgagggactgtaccatagagcttatcaaccaatcaggatcagccaaggataaagtaattatcaaaataaattatttgctattaaatcttatgaagtaaaaaggtcacatatgttgacattaaaggtcaaatgcacaactttctcatttaggtatgtttgatagagatgttagagaggagagaattacagtgaatggaaataaaaaaagaagtaaaaataggtaagaaaaaagtgaaaattaaatgtatttgattgaaaagaaacagaaaataagttagagagatagaaagatgaacaaaaatatgtgacaatagtataataggtctcaccatttttaaaattaattttacctttccatccaccctttctctcctaatttctcactgtctcgcataccaaacacgggtaaagtaaaaaattatccctcaaaattttctttaggccagccctgcaaccaaatagaccttcacttgtgttgggatccctccttaattggagaggttctagaaaattctagagctttctggaaaagtgtagaattttctagaacgtcctggagaactctagagtagtgtagaactctctagaagcttgtggaagaatgtagaaacctctggaatattctggagatgtgtggaaccttctagaaccttatggaagagtatgaaaggaagcagaagagtgtagagattcctagaatgtgtggagtattctagagaattaatctccaccctaggatacaagtaatctccaccattcattgtggaggtggagtagtataaataagggtaggagccttcattcctatccatcccagacaagagtgaatccacttcttagagtgagaaagagcctctttgagagagaagataaatagcttgggaagtctctatcctcaagcttgagtgagccaccatagagtgagtcaattttgtaaacacatccttgtaaccctactattactttgtatagtggaagaatctccatattggagaattataatcctgtgctcccattactacctttaattactaagtgcctatcttaacttcacgaagcgggaaagtccgagttttcccaacaacttgctaaaaaatattcttaatttgcatgccaacataagcgacaatcacttataatctcagtaaaatttgtgcttatcacacaggcttaagatttcatgaggttaaagatttgccaatcaaacgtgttgagaattcaaatgtgaaacagagttgtgttaaaatttgttcttatcacccctgacctataaaaacaaatctataattctattgataaaacctatcattcatgatgcatcaaatttaatttatctctcttatgaatgaaaggaaagaaattggaatagacccagagaaattaagcagatgggtgttggaaaacctgtgacaaaataggatctttcaaagtcttgtttagtccacaaccgcagcacgatcaatggtctgaagaagcctgtgtgcatggaggagcatcctcattagacgctgaacggaaaacggaggcagcaatgccaagaagctcggagctataccatgcaagctttattcggattagttccactgtggattctaaggtcctgcagcttctgttgtttcggtggaaaaatcaaagagctatggtgacttctcttgaatgaagtcaaggcagctaatgccatgacacaacacaagacaatgtgagaggatcaaaaggctgtgtaagagagggggaaattgctatcttgaaccttatcattgtatgttacaaattttgtttaaattaatgtgacattatgtgactaattcatacttggtttcataataagcatcgatatagcttggtgttattttccttgagcataatgaagtcacatttttcaactacctttagcatcaagcgtcaaagaatttaaagactttgataatctcttgagttatctaatttttcttacatgatagagggatttaactatttaaaaaagtttaaaaatcatttatttatttgtgtaagtcatgattatgtgactttttttccctaagtctaatttgttttaatgttttagttttagtaaagtagttaattttaataaataaataaaaacttattaacccctaccccgcaaatcaacacgtattttgactctggatgaatcgaaaatacgttacttaattagaaaaatgtctaacatcatgtacgtttctcttatcagcttaaccaaggtaccctactccaaatctccaagcaacgttttggatttgacccagtcagtttcttttctatcaaaagctccaatgaagttcgttcggcaacagattggtttattacaacaaacaatccgtacagtacaatagattgtctgcacatgtacaactccagttaggtcccacagttctatatgatttgtcgcacacatccttctcatctgtgccacacgcccaaccaaaattccaagccccctttcattccaaaattctagaattattttaacttgattgacttcaaaatctttaatttaatcttgaattaaatacttaagtagtaaaattttatctttgaatcatctagcacatgaattaattgtagaactgttctaacttaaccaatgattttgaatttaaaatctaattttaccaattagagaatgactatttctcccaaacatacatataatctctattaaaaaaagtttaacctttgaactgtttaaagcatttgcatatttcaatttaaaattcaaactcaaaatcaaaagacagttaaaacaatctcgaagtcgaactagttgtttgatactatttgatcattaagtaaactagtgtagaggtgtgtggtgtttgattttgttttggtggagttatgagttatgaggaatccagcgcagcaattaaatggctgtcgaaaagcaatatatagagttgtattaaatgcagcagctaattacccccaccaacttgctttggccctctcaagaaaacataacaacactcactctaatttccctcccttttccttccacaaatggagaattccccatctcatccttcctcttcttctcttcaatgtcctccttcaaccccttctctcctcctccacccactgccaccaccaccccttacctcaccaacctcggcttcggctactccatcgccatcgccctcggcgttctcttcctcctctccaccctcatcctctcctcctacctttgctgccgcaccctccgccgccgcaacaacaacaacaaccgccgccacacccccgatggcgtcgtgctcccgcgcgtgatcttcgtacccgaggacgaggatgacgacgacgacgacacccgccagaacgacgtcgccaccggcctcgaccaggccgtcataaactcctaccccaagtttccattcgttaaggaaggtaattacgacagcacatgctcgatctgtttgtgcgagtacaaggattcggagatgctgaggatgatgccagagtgtcgacactactttcatctctgttgtctcgacccatggctcaagcttaacgggtcctgccccgtttgccggaactcccccatgccgacgccgctgtccacgccgctgcaggaggtcgtgccgctctcgcagtacgccgatgccagggcgaggaggtgaatttgaatttgaattttcttctccggtgtagtttgagttcagtggggaagtaaaatgagaagcatttaaatgtttttagttgtttttcttatcaaaattcgagttttatctcgaccatcataataaaaagtagttcatgtttagatgttggtgtagattgtctaggtaagactacaattttgattagagaatagtactaaaaacatttaagaaaaaaattgtatctaaattttctgcaaaatgtaaatcttccctgtagacgggagatggtgattctttttttttttaatttctgaggaagaaaaatgaggtggtggtggtggtgggagcatgtgtaggattacaaggcaatttcaggagggtttgaattttgattatgtgattctatggtgttagcaagtgttttgcgggttactgagttatttattggtaatgcacagaaatgccgaatttcacttttttagggaaaattattcgcgactgcgttttgtttgtaccatgattttttatttgacattggatataatggatacttttttccctagttttgttggtttgacccacagtgtgacggtggaatgaatctcttggtgagaacattagactctgttgttttacggggttttagtktttttttttttaactttctcagagaaattggttgtagtacctttttgtttcccgtttttcaattcatggacccattgggttgtcgtcattagtagacagcttgcacttgttgctcattcattcccgggacaaacaaggtcttctgccttttttytctttttttggcacaatatttacggtgtcgtattcagaatagagaaagtcaatgaattacggattcgtggcacgggtatgagctagctaataacgtgtgctcgtgagattaccaagttttatgctaaccttgcttcagatgcactttatctgtatctaaatatggtgccacatgcatcaactcattctttagtgctttctatccttaatgcatttcctagcgctattggcatttcctctagattcgggcaagagttgaatcacaaattgttaatatttcaaacaagttgtgattacctgaaatagaagataggtttgtgggtgacaatcacttttttgcatattgatattaagtatttgttgagcaatcactaaaatggtgtgtcaatctctgtttattagtatgcgtgggatatgtgtcaatttatctctgtttatttgttgagcaatcactaaaatggtgtgtcaatctctgaatcctgaggtcccaaatgggaatcaaaatgaggtgctgatgaaaactgttgaaatggtaattcagaaaattgaagaggaactggacaaactgagggaaacaaaacaggatccatcattgaccccatctgtattccagcaaggtgcatttctttctgatatattaggacttcttgagtcggcggcagcgtctcatcaacaaaatgaaggcaataatcagcaaaatggtggtttcaacaaaaaagaagcaatctgatgagcctgctgactactttctgcgagaagagattcgcatgttaggatcaaacctaacagattaggaaaacaaaattttatgggggaaaatgcaagcttcaccagcataggacacacgtgctttgccatgaaggaagagctagaagaatacatggattatgatgttggtgagatctgcaacgatgactggaaactggctcaaaagcttatggttcatggctgtgatcctttacctaggagaaggtgcttttcaagatcccctaagctatacaaacaaccatttcccgtcaatgaatccttgtggaaactccctgatgatagaaatgtcagatggagtcaataccagtgcaagaactttgcttgtcttgctggcaatgccactcgtaagggattcttcaagggtgctgattgcttcaacctcaccaatcatgagatgccaagatgggtaagcctggaagctgattcagaccgcacagcagattttccttataggattggacttaagtgttggaactgggacttttgctgctaggatgagggaattcaatgtgactatagtttcagccaatatcaattttggagcacccttcaatgaaatgattgctctaagaggacttgttcctctctacttgactataaaccaaaggcttccattttttgacaacaccctagatttgattcacacaacaaggtttcttgatgggtggattgatcttgtgctccttgagtttatattgtatgattgggatagagttctgaggccaggggggttgctttggattgacagctttttctgcttgaaggtagatttgtatgattacttgcaggccttcaaaatgctgaggtataagaagcacaaatgggtggttgttccaaagcttgataaggatgagcaggaaatgttcttctctgttttagagaagcctcataggccattcaggtgatcttccattatagtgttgacttcttgcctatatggttcccatcattctttagcatatttcatattcattaattttgaatattttacagttgtcttgtgtattgtgatcataagtttttagaaatgaccttttaatatcatatatgtcaataaggagcttcatagttcaatatacttcaatatcatatatgtcttcgttgttgttgattttgctagtattctctctgttactcccattctacattatgtactgtttatgatgatatccaataattctaggataaattatgttgctataattcagcctcgcagttgcagtgaaagcaattcatatatttgcttcgctaccagaacaccataatactacaagattttcaaaaacgtctctggtcagtggcataatcctattaattcattcaagtcttcgagtcattcaaactaagaagcacgagcaattccaaatagtaactagtgtgatgcgcaattcagatacgaatgtatattatacaattcccagatattttgaatactttagttacattttataatatttgtattattattaacaatatcataataaaataaagatttaataatatacaatgattcataattacttttaaaaatatcaattctattttagttgaaaatttacgttttatgttgtttttatattataatatggtgttttataatttttaaatttaccgtaatatcatattcatctttcctgacaaaacgtgtgatttttatgtaaaataaaataaaaaaggtgtaatttttaactggtcttggtcacttcatataatatataaaaaaaattaaggttggaaaaggatggggggagtaactttcacgcataaggaagaaattgaggaacacttccacacaccaagtgaagagtcacctagaaatcagaacaacaaaacaaaacaatctagtgtatgtttgatttatcattagaagatcctaaactgtcgttgaattaaaaattatgaattcttagttacaaaatatgcatgagwrkgakmmycgkgtryykagytcgaacctaacgttaaacaaaagacacccattagacatcgactggaatgggagagtgtgataggtcctgcagcaatgaaaagtttttgtagagcgtcaaatatattggttgcgtaatcgatggtccagatgtagggcaattaacgtgtatggaatcccattttttaaaatagttaaatgtttgtgaaaatctttatggagcccacgtgttgagtgtacgctaggcaggaaggtgtttgcactaattgtgtttggtgtttcatgttcagatattggaatcattttaatttaataatcatttcgagtccatgtacttcgcaaaatttctatggataatttgggttaattgttgtgttgagagtcattttcaggcccaaattacgaatattgttgaatttgtaatagttaaagttaataatatttgttatatatgtagtaatttattaaagaaaaggttacaaatatttttaaaatatatacatgttacaaattattttaaaatgaataaagtgaatttgtttatgttggttatgttagcttagagaaactcaaccaagagaggtggatgattcactatttgagaatttatatcgagaaatttgatgtcaaaatcatttatgtttttaaggaatttggtgtcgaaatcctttataatgttgattctcaattgagatttattattaaatcattgtaatttgttctattagcctatatgcatcctttaaattttaagtggaaatttgatcctaaacatagagaatcatagaccgattgaaagtaaagatgtgaatgtttacatgaaagaaaaaaaaactaaagatattttatcatgttctattttcaaatgaaatctacatgcaatatggcaaaaagcaacaatataaagttggttgtatccagtagcaatacattagggttgagattagtgtttagggtttatggttacaatttaggattaatattaaggtgaaaatgttatggtttagggttgggatcatgtttggttaagggttatggtttaaggttagtttctaaggttaggattagctttaggattagtgtttagcattaaagggttaggtttaggagaagtgttagtggattggcaaattagtgcttaaggatagggttagggttcgaggtaagggtcaattttagggttagggtttaaggttgttttcaaggtgatattggtggagaaaggtgaagaaagagcgggagacgaaggtgaatatggggtcatcacaagtattaggtgaggcaaatatgggtcatcagcagatggaggctataacatctcaatcaaatcacactagaataagaagaatccaaagaatgtgtagatatgagactgtacatatgaagatgacttataggaattaattggtattacctataccaacaagatacatttactttttagttcctcatcacttaaaaactcaacaattaggagtgtttgacttggagtagatatgtgatgggtgaccgtctggaaaatttctcagaaagcatgtgagtgaggacaaagcactctgtaaagtcttgtgttggtttacaggatcagtcattaatcttggaagcaggaagaattaatttttgaggtctttgaaaggactacctatgaaggattctaactaacaaggatattgagtaaagtgtcattgcataaagtattgtagtgcgagagtcgtaaaaaagtaaaaaatcaaggatgttacaaatgatattaaagtaaacatctctttagtacagtgtgattcaggggacgaaccaggcaaaaaatggtgggtgtgtaacaccccaacgaagcacaccaaaaagagagggatccagaggctatgtaattatgagattgtatagttgaggatgagtgaaagaaattaattgatattacctttaccaaaaagatgcatctacttttcgatagtcaatcacataacaattccattgttaagagtatttggcttgaaatagttatgggatgagtgacatctcaaaaagttttccgaaaagcatgtgagtaaggataaaacacactgaaaagttgtgttggtttgttggttcagttattgatcggggaagcaagcaaagttaatttttgagatctcaaaaaaggcttcttatggagggttctgactagtggaggattctaaccaacaaagatgttgagtgaagtgtctccacataaagtgttatagtgtgagagtcaccaagaagtaaaaaattagggatgttacaaatgatatcaaagcaaacctctatcccaatatggtgtgatttgagaacgaactagacagaaattgatggatatgtaataccccgacgaatcacaccaaaaagagagagacccaaaggttgtgtagctatgaaattatacaattgataatgacttaaagaaattaattgtactacctataccaacaagatgcatctattttttagtagtccaccacttaagaactccacggttattcatgattggctttgagtaattatcatgagtgaccttctgaaaaatttatcgataaaaggtgtaaataagaacaaaatacgttaaaaagtcttatattgagttataacgttgatcattaattctgaaaataaacataactaattttttaggtctcaaaaaagactactttcaaaggattttgactagtggaaaattttgattaacaagaatattaaatgaaatgtcactccatgaaatattacaatataaatgaaatcaagtataaacttaaaaattataaatgttacaaaaacatgatgaaaccatcgtaaaatagaggaaatgaccaagaaaggtgggagtataagaatgtgattcattgttttttaaacaataaaaattatattgaaagttaaaaatcgatacttttatatattaaaattattagaattaatcctataccgatgtcattttcaattttgaaaaatattgatgaattattaaatatgttaagctcttattgaaaattttaaaaatatcaaaactccattgaacacgttaatatagttaaagagctaaattgagtatttagctgaatataaatcatgaagattgtatttgcttgcttgcttattagtatttgtgatttgcataaacgagaaccgatccatgaccccgagtcagtgatgtgtcctaatcagtagacgccatctgtcccacagcattttatattgtcggtcactacttttgaaaagcatccaccgccatgagtgagtgtgactctatacgtcactaattctctgacaaccaattacttttttttatttgaccataagaataaagaaatagaccattaaatttgcataatttcaatattttaaattactctctcgctatttattagtattaccagtatcgaaatactttattttaaattacttatcattttaaatattaaaaattaactaaatttttatgatacttttaacataaatatttttaattttactaataattaatttttatttttaaaaaactatctaataatctttaattcctctctcattatattttttattcacaatattataattttagatattaattaaggatccaagctaaattatatttggatttatgatataatagaatatatttattcattaaaatttaatttaaaatacattaatgagtaattttgattttatactttagtcctattagtacattacattaataaataaggtatattattttaaaaatgaatataattaattattttcttaaaatttactatattttcttaaatgaaagataaaataatatagaggaaataatttatttttattgtataaaattaattatagaacaaactttttaacataatattttatgcaattttagtatagtttttaattaaattactttataaaacaatgtatttaagctaaaagatatttattttaaaaattaatatgattaatcatttttttaaaatgtatatattagcttaaatgatagataaaataggatacataaaataatttgtttttattgtataaatttcgaattaagtataagaaaaattttaatatcagatttcatgtagtttgagtatagtttttaattaaaatacatttataaaataatgtatttttttataataaaaatattttttaaaataaatatgattaattattttcttaaaacgtatatattaccttaaatgatagattatataagatagagaaaaaaaatatttttattatataaaattcaaattatttatagaaattttattaacgtaatatttcttgtagttgtagtatagtttttaattaaaattaaaattttacttaaataatcagtattacctattatatcaaaattaattatgtgaattaccaatataaaagtttaattgagtaattatagagcaaagatttaacatgacatttcatgtagttttagttaaaattaaatttttacttaaataatcagtattacctataatatcaaccttaatcatgcgaattatcaaaataaattttaattaattaattatacataaaaatttaacattttagtatatttttaattaaaattaaatttttcataaaaataaatattacctataataccaaccttaattatgcgagttattaaaagaaaattttaattttaattaaacaatattaaacatattcaaaatattatattcaaaattactcctcaactaatatgctattgcaacgtaaaaattgtattttagacggtcagtaaactttaattgtattttaattataatattttttgttggtataatgagaagaacaaaaaaccaacacaaactaagcaactaaaatacaagtcacgtaagaacgtggcaagcaccgcttgaggaagtgcgtcatagaaaatattaattaaaaaaaaaaaaattaatttttcattaaaaataaaaaaatactcaatatattatattagaaaatattccctaagtaatatgcttcttttttgtgaataccctaactaatatgctactgcaacataaaaaaaattattttaaactgtcagtaaactttaattgtattttaatttcaaaatttaattgcagcatataattttgaatagcaaacaccgttaatttcaacggcgcgaaaaaacctactgcatcaaaagtcaaacggtgtctcctcttctcttccctctaagtctatcatattactcgtgccagattcaaatgtgtcccgagcctgagcttcgggacccgaacctcgtcatgcgcaacactataaattgggacaccgagcgcatcgcacgttacttgtacttgccctcagcaccagccgaaagccaagaaaaagctttcgatccctgtaactgcgttcgactatttagggttttttcggagacaatggcggatgaatcggagcacacggcggcgccggctgccggcgaatcgctgctcgacaagatcgccgagaagattcacggccacgacgactcttcctcttcctccgattccgattcggataaaaaggaatcttcgtcgattaaggagaaggttttccgcctctttggaagggagaagccagttcactccgtcctcggcggcggaaaacgtatgactctctctctctctctctttatggacactttttagtctacttgtgtttttggttagatctgtgatgtgagatgattaagtggagttctctatatatatgtttcgctgatcggtgttaagttgattttaaatgatgctacattttcttaaatttctcaggatttttatttaagaaaattattaggaagtcgagttaatgtgttgaaggtttgcttatgtcgatgctggaaaagtaggttttgaatgctgcattattatttggaaggggaattcgagttcacttcaggtttcaaatattttcgtgtccaatgttcactgatactgaatctgaagagcatttttatggttctctagtggtgtttttcattgtgattatttattgattgagtcctattcatgtatggcattaccatcaattaatttgtggttttaacatgtagatcgatgatatctttgtattgggcaacttgtggaaagacgtgatcatattttcactgcctctgctatattgcattttattttgcataaacaattcttatttatttgttgtgaacttttgatttacttcctgagatgtaagacgatttttgtgcccatttgttacagtctttccataaaagaattagttataagaaatattcattttatttttaatttttcatgtgtttgtttcagcttttcaaaagaataaaaaaatattttaaattttttgtttaaatcagaagctattgaaaaaaaaaagtgattttataattgtgagcaaacatgaattagtttcctttagatctaaaaaataatccctgaattaattgaaattagaatgagaattactttatgtttaatttttaatctgtaaccaactagtctttattttattttttaataataatcttcaattgcagctgctgatgttctcttgtggaggaacaagaagatatctgctggtgtacttggtgtagccacggctatttggatcttttttgagttgcttgaatatcacctgcttactctagtctgtcacatttccattctgttgcttgcggttttatttttgtggtccaatgcccatacctttattcacaagtaagactctaggactcattgatctattgtgccaaggcatagttgcctacaggctatgattatttccaaccaatttgcatgtaatatcaaataatgttgcagggcaccacctcgcatcccggttgttcatatccccgaagaaccaattctccaatttgcatctgcattgacaattgaaatcaaccgtggatttgctgccttgcatgctattggttctggaagagatttgaagacatttctcattgtatatatcctgcaaacttcttatttcattaatgcacattaaatgttatttttgtatgcataatcttcatgaaaaatatgggctaaatgttaataacttaatatcccaagatgttgacttggtgttttaacatgcattggactacaaattgccgattccaaatagtgttagtttagctttcagtaagaactgaattattggtgtcatgtcattgtcttatatgagtattctgttgttgatgttatgttaggttattgttgggacgtggattatatcaattgtggggagctggtgcaatttcttgaccttgttctacattggtaagaacaatctattccgtttcatatataatgttttcaaagttatgaatatgaactatgaagttttacttatttcttcatttgcagcttttgttttgttgcatacggttcctgtcttgtatgacaagtatgaggataagatagaccctctagcagagaaggcactcattgagttcaagaagcagtatgctgtgtttgacgaaaaggtcttgagtaagatcccgaaaggcccattgaaagagaagaagttagcttagtgtttaagtatttgtactctcatcaagaactaaaaaggaggctacaacttcagtcttttttatttactagagcctcgtttctttcaaaccatataccaagccttgttttggattttctttaggcgggtgtggtactcgcaggtttagcttcaagatgtagcttttgattttgctataagcatgtcgattttgcttattacagtgtttgttggacttgtagtcttcaatggacaattaatttgtaagcaagaaatatttatgttaattgtcaacgttatttttcattgatgaaaaattgcatggtgagtctacggtgttggtgtgattcatggtattattttaaatcgaggtaaaatgtcatctattgaaggtgatgttatcttcatggtatcctaaatcccaagaaccgaataccatgactcacaagattaataaaatattttattataaagacataatttgattaacaatttttaatatttctttaattaattttttacttttcactatgtcaacctattatcaaaggaagttacgacactttatattagtcaacgtattttattaaaaagacataaattgattaataaattttaatgtttttaattacaggtaatttatttttaattaattaaatatatctaaagagtaatatataatataaatcttggcaaatatattgcagtaaaaaaaaaaagttagaacatctttaatggaagatgttgagctcaagatttgattttcttcagatcttctacgaaagcaacaaaaataaaaaagtcaaaggaagcggagaagaagatgaaaaggagaatgaactggaaaatccaaaggagagtgtggaggaggaggatggtggcacagtgcgcctcgtggagggaaggctggtggaggatgagggaaggaaaagggaattctgtgacttgcagaaagtgtatgtgacaaagtaggcttctaccatgcatgatagaggttgtggaaaaaattaaaataattaataaaatatatttaagtggggttagaaccttgagtattaaatgagagcattattttaatgcactctccttatgtttttgacaaactttttttttgaaaaacgtttatttaaatattaataagtatatattggcaaaacattaaatataatgtttttaatgaaaataaattgttctttgattattatttttaaaaatggtaataaaccaaatcattaataaagtttaaatatttatataaagaaaagaaaaaagaaaaataaaaaagttggactttattaatccttagatttttttacttaaaataaatttaaaaattaaaaatttatttaaatatattttttctaattaattatgaaggggagaaaattagtttacttttttctgttttttaatgattatcgatttaattttttcttgtatggttgtatgcaatctttactttcctgtttccatatgactaatttaaaatgactttaagttaagtaagatataagaattattagaaactcacaataactaatggtttatatattttaaaattacaatgagtcaaagataaccatagtgagttacaataaggccattcaccatgtttacaatattaaatgatacattaaaataaaatattttaaataaaagtatacatattttattagtattataatattttgaatttgacaacagtcaaggtcacatgagttttgtaaaagactaaatagaaatgtttgaaagactttttaatgattgaaattcattagtgttgaataatttaatgttaaatataaaatacattaattaatataataacgacgacttttcaatgaatcattattgtggattgtgagaaagcatcccatctgtcaaacataataaatgatctaaaataaaatatattacttacaataataaagacttttcaatgacttattaaacaataaaatttagattgggaacaaatcttcaaagcatttttattttattttaaataaatatataatagtttttatttatttattttatttttaaattctattttaaacaaaaaaatatctttattgaattttattttaaatggtgattatttttttgttttccataatacccttaaaaatatttattttatttaatcagtaaaatattatttttagtttattttttcttctaatattattttaaaatactcataaatataataaatgaactaagatattcaaccatttttaattataataattttattttactttgatatattatttaaagttttaagcaaaatcactacggacaggcctagagaagtaaagtttaagtattttacaaaaaatcttaaattctatacttgttgccactgtaacactgttgttgtacataagaaacaaatgtttttagcatgtgatatttatcgattatccaaacttaaaattaacaagctgaataactcaactacaaaaaaactatcatgaaatatattgaaacttcaaaatcaaaatagaaaaatattaaatatgatttaagattcacacaaaaaaaattattcaaaagtattattagtaattctcttaaattaaagtcaatgtcgttacgaatcaatattgtttagaataaatacgtcaagatactcataagctatgtgacacttttgacactttgggagggaaaaagataaaaggataattaataagtgatatgatttaataaataatataaaaatatgtatttgaaatttatgagtgtctaaatataaaacactttattgtttattttgcacgtcatactccccattgagtagtcagtgctctctggtcacccagcacagtaagtttaaggttggtctcgatcaactccaacacattttaatataactaatacttttgaatgtgagttgcatagaataaatatttattttatagaattaaaatttataataaataaatatttatatgtataacttatattataattgaaaattataatttatataaaaattatttcgaatacataaattaattatttgaattgtgatatagttttacttttaattaaggatgtatatttttttaaaatgttaaaactaatttacatacagatattaaaaatttaaataaaatcaatattattttgagatgattatctaaaaaaaaggaatagttgtgtgaaaccaaatgatatttcacttgggtcgccaaaaaaaaattaaaaaattaaaaataaaatattttattagaaatacataaatgaaataatctttaacattttccatttaatttattacaattcactctgtcaattttttagaaatttctttgttatatctaattaattgggtattatttcaattaattaaatgtattcaaggcatactattgtatagaaatcttgacaaatgtataacatcaagaattttaaatcttgaattttaaatgtgtgcactagtttaatgtttttttccttttttttttatcaagaaataccattattaatacatttgataaaaattttaaataaatatttttactgaaaagataataaatatgtattgatataatattaaatataatgtttttgataaaattattttttaatttttttactattatttttaaagataatagtaaataagttattaataaaaatgaattgtttatataaagtgaatagataagtaaaaatgaaaaaaaaggctttatttttcattagattgaataaaagtaattttaaacataactcgattaaattaacctatctctaattttattttcaccaaaaatattctttaatttttcactttatacattttttcctttaaataagttaaatttgagattaaatttattatttaaaaaaatttagcaatttatttgactactattatggaagttgtgtgatgttcgggcaatacattcatcttgataatattatctattttattttttattttctaattaaatataaaaatgaaaaatgaagtttatttttttttctttttctttattgctatccttctatcaatttatttttccttctttggttgtatgtaattttgttcccttctttcaataatcaaagttaaatgtcccatttaaataggttaaggacagttataaattataaaagtaagttactttttgtgtgggatccccgggtaccgagct

SEQ ID NO: 18 - DNA, 20 bp, Soybean
tctgtggtggcacatcgatt

SEQ ID NO: 19 - DNA, 19 bp, Soybean
tgccggtgctaccattctg

SEQ ID NO: 20 - DNA, 15 bp, Soybean
aaaccaccgagccag

SEQ ID NO: 21 - DNA, 15 bp, Soybean
aaaccacggcgccag

SEQ ID NO: 22 - DNA, 28 bp, Soybean
cagatgaagacccaatgatatgtgagat

SEQ ID NO: 23 - DNA, 23 bp, Soybean
gggtgccactgtcttgtttaagt

SEQ ID NO: 24 - DNA, 16 bp, Soybean
cacatgcagccaagca

SEQ ID NO: 25 - DNA, 16 bp, Soybean
cacatgcacccaagca

SEQ ID NO: 26 - DNA, 30 bp, Soybean
tggtgtttattttcgaccaaaattgaagtt

SEQ ID NO: 27 - DNA, 33 bp, Soybean
cttacatacaaatctttaggctccttataacct

SEQ ID NO: 28 - DNA, 23 bp, Soybean
ataatctacatgtaaacatctat

SEQ ID NO: 29 - DNA, 23 bp, Soybean
ataatctacatgtaaacttctat

SEQ ID NO: 30 - DNA, 23 bp, Soybean
ggacttggagaagaaattagcca

SEQ ID NO: 31 - DNA, 21 bp, Soybean
gcaacatcaaaggctctcaca

SEQ ID NO: 32 - DNA, 23 bp, Soybean
acagtatgaccagcagcttccaa

SEQ ID NO: 33 - DNA, 28 bp, Soybean
cgatgaaatatatccactcttattagca

SEQ ID NO: 34 - DNA, 30 bp, Soybean
actaaggcacatattctatataaaaaaact

SEQ ID NO: 35 - DNA, 35 bp, Soybean
ttgtgtattactaattatatcatccgtgaaaagct

SEQ ID NO: 36 - DNA, 25 bp, Soybean
agataacacatttcagcggctttcg

SEQ ID NO: 37 - DNA, 24 bp, Soybean
tgatgatggagttggtgttgcagg

SEQ ID NO: 38 - DNA, 25 bp, Soybean
cttcacattggccaccacaaccaca

SEQ ID NO: 39 - DNA, 24 bp, Soybean
gcaagctaaacatgattgaaggat

SEQ ID NO: 40 - DNA, 28 bp, Soybean
gttttgcctgatttattcactgtttcaa

SEQ ID NO: 41 - DNA, 35 bp, Soybean
gttggttttctacggaatggtagtacgccatccat

SEQ ID NO: 42 - DNA, 29 bp, Soybean
tcttggcttgtcttctatcttccaaacga

SEQ ID NO: 43 - DNA, 30 bp, Soybean
agattaaacttttgggctatgaaacccaga

SEQ ID NO: 44 - DNA, 35 bp, Soybean
aatttcccaaatccatatgtattgtaccgatatca

SEQ ID NO: 45 - DNA, 24 bp, Soybean
cattcgtaccttcaccgcattact

SEQ ID NO: 46 - DNA, 30 bp, Soybean
aagacactatgaatccctaatctcatgcca

SEQ ID NO: 47 - DNA, 35 bp, Soybean
aaatagataaaagattaaaataaattttttaaaag

SEQ ID NO: 48 - DNA, 30 bp, Soybean
aaaggaaactaattcatgtttgctcacaat

SEQ ID NO: 49 - DNA, 30 bp, Soybean
tttgtgcccatttgttacagtctttccata

SEQ ID NO: 50 - DNA, 35 bp, Soybean
tattcttttgaaaagctgaaacaaacacatgaaaa

SEQ ID NO: 51 - DNA, 24 bp, Soybean
aacctgaattgccagcataagggc

SEQ ID NO: 52 - DNA, 24 bp, Soybean
aagcgaggaccacttctgtgttct

SEQ ID NO: 53 - DNA, 22 bp, Soybean
aggagatgtcatcaataaagcc

SEQ ID NO: 54 - DNA, 21 bp, Soybean
tgctgccttgtctagacctaa

SEQ ID NO: 55 - DNA, 24 bp, Soybean
atcagcaaaacagatgcagacgtt

(19) United States
(12) Patent Application Publication
(10) Pub. No.: US 2013/0198912 A1
(43) Pub. Date: Aug. 1, 2013

(54) DNA SEQUENCE THAT CONFERS APHID RESISTANCE IN SOYBEAN

(71) Applicants: Matthew Hudson, Champaign, IL (US); Brian Diers, Urbana, IL (US); Ki-Seung Kim, Urbana, IL (US); Jianping Wang, Gainesville, FL (US)

(72) Inventors: Matthew Hudson, Champaign, IL (US); Brian Diers, Urbana, IL (US); Ki-Seung Kim, Urbana, IL (US); Jianping Wang, Gainesville, FL (US)

(73) Assignee: The Board of Trustees of the University of Illinois

(21) Appl. No.: 13/577,227

(22) PCT Filed: Feb. 4, 2011
(86) PCT No.: PCT/US11/23764

§ 371 Date: Sep. 17, 2012

Related U.S. Application Data
(60) Provisional application No. 61/301,991, filed on Feb. 5, 2010.

(51) Int. Cl. C12Q 1/68

(52) U.S. Cl. CPC C12Q 1/6895
USPC 800/302; 536/23.6; 530/378; 536/25.3; 536/24.3; 435/6.11

(57) ABSTRACT

Provided herein are isolated nucleic acid molecules representing a genetically defined region of the genome of the aphid resistant soybean plant (Glycine max) cultivar Dowling that confers resistance to soybean aphid (Aphis glycines). Within the region is a gene encoding the aphid resistance protein Rag1. Rag1 aphid resistance amino acid sequences are also provided. Also provided herein are methods for conferring aphid resistance on a plant or enhancing aphid resistance in a plant by transforming it to contain and express such nucleic acid sequences encoding Rag1 aphid resistance or introgressing DNA encoding the trait into the plant by plant breeding. Further provided are polymorphic markers useful for identifying plant germplasm containing aphid resistance, and methods for makings such markers.

### CROSS-REFERENCE TO RELATED APPLICATIONS

This application claims priority to U.S. Provisional Patent Application Ser. No. 61/301,991, filed Feb. 5, 2010, incorporated herein by reference to the extent not inconsistent herewith.

### STATEMENT OF GOVERNMENT SUPPORT

This Invention was made, at least in part, with U.S. Government support under U.S. Department of Agriculture Soybean Disease Biotech Center Contract No. AG MH 2008-04976. The Government has certain rights in this invention.

### BACKGROUND

Soybean is one of the major crops in the United States with 74.8 million acres and $36.4 billion farm cash receipts in 2008 (USDA. 2008). However, since it was first detected in North America in 2000, soybean aphid (Aphis glycines) has become a significant risk to US soybean production. The soybean yield losses can reach 202 kg/ha when the aphid population reaches several thousand aphids on an individual soybean plant (Patterson and Ragsdale, “Assessing and managing risk from soybean aphids in the North Central States,” Crop Sci. 44:98-106 (2004)). Soybean aphids can reach such densities because of their tremendous reproduction potential, with 15 to 18 generations on individual soybean plants in a given season. Aphids have a tube-like mouthpart that sucks juices and nutrients from the plant. Aphid-infested plants become yellow and stunted, reducing pod and seed production (Sun, Z. et al., “Study on the uses of aphid-resistance character in wild soybean, I. Aphid-resistance performance of F2 generations from crosses between cultivated and wild soybean; Soybean Genetics Newsletter 17:43-48 (1990)). Soybean aphids can also transmit viruses, such as soybean mosaic virus, soybean dwarf virus and alfalfa mosaic virus (Hill, J. H. et al., “First report of transmission of Soybean mosaic virus and Alfalfa mosaic virus by Aphis glycines in the New World,” Plant Disease 85:561 (2001)). The infected leaves are distorted, wilted, wrinkled and puckered, which affects yield significantly. Therefore, aphid control has become a major concern for soybean growers in the soybean-producing regions of the northern USA and southern Canada.

Soybean aphid is a new pest in the USA, and its control has been limited to the use of insecticides. The use of insecticides is well known to increase selection pressure for insecticide resistance in the pests, to reduce natural enemy populations that provide biological control to aphids, and to substantially increase input costs for growers. It is estimated 12 million acres of soybean were sprayed with insecticide in 2005 to control soybean aphids. The most cost-effective and environmentally-friendly method of aphid control is to identify and use aphid resistant genotypes to protect soybean production.

In the USA, some sources of aphid resistance that have been identified include ‘Dowling’ (PI 548663), ‘Jackson’ (PI 548657), ‘Sugao Zarai’ (PI 500538), PI 567541B, PI 567598B, PI 567543C, and PI 567597C (Hill, J. H. et al. “First report of transmission of Soybean mosaic virus and Alfalfa mosaic virus by Aphis glycines in the New World,” Plant Disease 85:561 (2001), Mensah, C., et al., “Resistance to soybean aphid in early maturing soybean germplasm,” Crop Sci. 45:2228-2233 (2005)). These resistant accessions have been classified into three different complementation groups indicating genetic differences in the genetic basis of resistance among these sources (Chen, C Y et al., “SSR marker diversity of soybean aphid resistance sources in North America,” Genome 50:1104-1111 (2007)). Dowling, a maturity group VIII cultivar released in 1978 (Craigmiles, J. P. et al., “Registration of Dowling soybean,” Crop. Sci. 18:1094 (1978)), was reported to have strong antibiosis-type resistance and can effectively control aphid population development during all soybean growth stages (Hill, C. B. et al., “Resistance to the soybean aphid in soybean germplasm,” Crop Sci. 44:98-106 (2004)).

A genetic study showed that soybean aphid resistance in Dowling was controlled by a single dominant gene, named Rag1 (Hill, C. B. et al., “Dominant Gene for Resistance to the Soybean Aphid in the Soybean Cultivar Dowling,” Crop Sci. 46:1601-1605 (2006)). To identify molecular markers linked to Rag1 for marker-assisted selection of aphid resistance in breeding programs, the genetic location of Rag1 was mapped to the soybean linkage group M between the two SSR markers, Satt435 and Satt463, with genetic distances of 4.2 and 7.9 cM respectively (Li Y. et al., “Soybean aphid resistance genes in the soybean cultivars Dowling and Jackson map to linkage group M,” Molecular Breeding 19:25-34 (2007)); Hill, C. B. et al., “A Single Dominant Gene for Resistance to the Soybean Aphid in the Soybean Cultivar Dowling,” Crop. Sci. 46:1601-1605 (2006); U.S. Patent Publication No. 20060015964).

There is a need for more precise mapping of this trait and for identification of DNA that controls the trait.

### SUMMARY

Provided herein is an isolated, synthetic or recombinant nucleic acid molecule comprising a sequence having at least or about 95% sequence identity to a sequence selected from the group consisting of:
(a) SEQ ID NO:17; and
(b) nucleic acid sequences encoding a polypeptide having an amino acid sequence of any of SEQ ID NOS:1-16 and combinations of said amino acid sequences.
   These nucleotide molecules can be used to confer, or participating in conferring, Aphis glycines resistance to a soybean plant. They can also be used as probes or computational query sequences to identify sequences in other plants that confer or contribute to conferring Aphis glycines resistance to the plants. The sequences provided herein can also be used in analytical procedures to identify subsequences that are significant in conferring or contributing to conferring aphid resistance to plants.

Also provided herein is an isolated, synthetic or recombinant first nucleic acid molecule having a sequence:
(a) wherein said sequence encodes a polypeptide capable of conferring, or participating in conferring, Aphis glycines resistance to a soybean plant wherein the complement of said sequence hybridizes under highly stringent conditions to:
   (i) a second nucleic acid molecule having a sequence selected from the group consisting of: SEQ ID NO:17;
   (ii) a third nucleic acid molecule encoding a polypeptide having an amino acid sequence of any of SEQ ID NOS:1-16; or
   (iii) a fourth nucleic acid encoding a polypeptide having an amino acid sequence comprising combinations of the sequences of SEQ ID NOS:1-16; and
(b) wherein said sequence is fully complementary to the sequences of paragraph (a).

Also provided herein is an isolated polypeptide having at least or about or 95%, at least or about 97%, at least or about 98%, or at least or about 99% sequence identity or similarity to a polypeptide encoded by a nucleic acid molecule provided herein, wherein the polypeptide is capable of conferring or participating in conferring Aphis glycines resistance on a soybean plant.

The isolated polypeptide can be a polypeptides having at least or about 95%, at least or about 97%, at least or about 98%, or at least or about 99% sequence identity or similarity to a polypeptide having a sequence selected from the group of amino acid SEQ ID NOS:1-16, including SEQ ID NOS:5-15, and including amino acid SEQ ID NOS:7 and 13.

Also provided herein is a method for producing a recombinant polypeptide comprising the steps of introducing a nucleic acid molecule as described above into an isolated host cell under conditions that allow expression of the polypeptide. The nucleic acid is can be operably linked to a promoter and then inserted into the host cell under conditions that allow expression of the polypeptide. The method includes recovery of the recombinant polypeptide. Conditions that allow expression of polypeptides are well-known in the art of plant genetic engineering. Suitable hosts are known to the art, for example the hosts can be selected from the group consisting of prokaryotes, eukaryotes, funguses, yeasts, plants and others.

Further provided herein is a method of generating a nucleic acid molecule capable of conferring, or contributing to conferring, Aphis glycines resistance on a plant, such as a soybean plant, comprising: obtaining a nucleic acid molecule as described above and modifying one or more nucleotides in the nucleic acid to another nucleotide, deleting one or more nucleotides in the nucleic acid molecule, or adding one or more nucleotides to the nucleic acid molecule to obtain a modified nucleic acid molecule, wherein the modified nucleic acid molecule encodes an polypeptide capable of conferring Aphis glycines resistance on a plant. Such methods for modifying nucleic acid molecules are well known to the art as are methods for screening and testing the modified nucleic acid molecules for their ability to confer, or contribute to conferring, Aphis glycines resistance to a plant. The methods include effor-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturation mutagenesis (GSSM) and any combination of these methods.

Further provided herein is a method for comparing a first sequence to a second sequence comprising the steps of: electronically encoding the first sequence and the second sequence in a computer program which compares sequences; and operating the computer program to determine differences between the first sequence and the second sequence, wherein said first sequence comprises a nucleic acid sequence described above. This method can include identifying polymorphisms between the first and second sequences that are diagnostic and/or effective in conferring or contributing to conferring Aphis glycines resistance on a plant.

The nucleic acid molecules provided herein and fragments thereof can be used as probes and/or primers for identifying a nucleic acid encoding a polypeptide conferring or capable of conferring Aphis glycines resistance on a plant. The probe or primer can comprise at least about or 10-12 consecutive bases, and in embodiments least about or 70 consecutive bases of the nucleic acid sequences described above, up to the size of the entire sequence, such as SEQ ID NO:17, wherein the probe is capable of identifying a encoding the polypeptide described above by hybridization, such as hybridization under highly stringent conditions.

In an embodiment, a nucleic acid probe or primer comprising at least or about 10 or at least or about 70 bases comprising a sequence of an Aphis glycines resistance locus of an Aphis glycines-resistant soybean plant, which sequence comprises at least one polymorphism with a corresponding sequence from an Aphis glycines-susceptible soybean plant. Examples of such probes and primers are probes and primers having the sequence of SEQ ID NOS:19-55. Probes, primers or amplicon sequences can be labeled as is known to the art to aid detection, e.g., with isotopic and non-isotopic labels. The label can be selected from the group consisting of a fluorescent molecule, a hemiluminescent molecule, an enzyme, a cofactor, an enzyme substrate, a hapten, and other labels known to the art.

The probes and primers described above can be used in a method for isolating or recovering a nucleic acid encoding a polypeptide with the ability to confer, or contribute to conferring, Aphis glycines resistance to a plant from a sample comprising germplasm of a plant. The method comprises: (a) providing a nucleic acid probe as described above; (b) isolating a nucleic acid from the sample or treating the sample such that nucleic acid in the sample is accessible for hybridization to the probe; (c) combining the isolated nucleic acid or the treated sample of step (b) with the nucleic acid probe; and (d) isolating a nucleic acid molecule that specifically hybridizes with the probe, which nucleic acid molecule encodes a polypeptide having at least or about 95%, at least or about 97%, at least or about 98% or at least or about 99% sequence identity or similarity to any of SEQ ID NOS:1-16 and having the ability to confer, or contribute to conferring, Aphis glycines resistance to a plant, thereby isolating or recovering a nucleic acid molecule encoding a polypeptide with the ability to confer, or contribute to conferring, Aphis glycines resistance from the sample. Or, nucleic acid molecules from a plant having aphid resistance can be amplified using polymerase chain reaction (PCR) using primer sequences based on the SEQ ID NO:17.

In an embodiment, the method for identifying or isolating a nucleic acid sequence capable of conferring, or contributing to conferring, Aphis glycines resistance to a plant can comprise hybridizing a probe comprising a nucleic acid molecule as described above, a fragment thereof having at least about 12 or at least about 70 base pairs, or a nucleic acid molecule having a fully complementary sequence to any of said nucleic acid molecules, to germplasm of a soybean under highly stringent hybridization conditions.

Also provided herein is a cloning vector comprising the nucleic acid molecules described above, as well as an expression vector capable of replicating in a host cell comprising such nucleic acid molecules. One or more isolated host cells transformed or transfected with such an expression vector are also provided.

Further provided herein is an array comprising an immobilized nucleic acid comprising a nucleic acid molecule as described above.

In an embodiment, a method is provided for producing a recombinant polypeptide comprising introducing a nucleic acid molecule described above encoding the polypeptide into an isolated host cell under conditions that allow expression of the polypeptide and recovering the polypeptide.

Also provided herein is a gene capable of conferring, or contributing to conferring, Aphis glycines resistance to a plant transformed to contain said gene, wherein said gene comprises a nucleic acid molecule encoding one or more polypeptides having amino acid sequences selected from the group consisting of SEQ ID NOS:1-16 and combinations thereof.

Further provided herein is a computer storage medium having recorded thereon a sequence selected from the group consisting of SEQ ID NOS:1-55, together with information identifying each of said sequences. The computer storage medium can be a processor, a CD, magnetic tape, or any other electronic storage medium known to the art.

The markers developed herein are useful for marker-assisted selection (MAS) of plants or plant germplasm with resistance to Aphis glycines. A method for using the markers for MAS comprises: (a) detecting in the plant(s) or germplasm(s) at least one allele in at least one marker locus that is associated with an Aphis glycines resistance locus flanked on one side by markers SNP46169.7 or KIM5 and markers mapping within 5 cM thereof, and on the other side by KIM3 and markers mapping within 5 cM of KIM3; and (b) selecting the plant(s) or germplasm(s) comprising the at least one allele in said at least one marker locus, thereby selecting a soybean plant having Aphis glycines resistance. In an embodiment, the plants or germplasm are soybean plants or germplasm, but they can also be other plants or germplasm, in particular species or varieties including members subject to Aphis glycines attack. In an embodiment, the allele is an allele associated with Aphis glycines resistance on soybean chromosome 7 in a soybean plant that is resistant to Aphis glycines. The marker locus can be a marker locus selected from the group consisting of marker loci identified by markers that are polymorphic in aphid resistant and non-resistant soybean, including ss107918249, 27A SNP456169.7, KIM5, SNP65906.2, 56B, KIM3, 21A, 25A, 83A, SNP7623, ss107913360, SNP442-1688, and SNP86377, and markers mapping within 5 cM thereof. In embodiments, the at least one allele associated with Aphis glycines resistance on soybean chromosome 7 is in a DNA interval having the sequence of SEQ ID NO:17. This MAS method can be performed as part of a method comprising further breeding to improve a soybean variety's resistance to Aphis glycines.

The further breeding method steps can be selected from the group consisting of crossing a plant selected by the above selection method to have Aphis glycines resistance with other lines or hybrids to form a first progeny plant, backcrossing said selected plant with said first progeny plant or progeny of said first progeny plant, self-crossing plants produced by the foregoing method steps, and combinations of said method steps. Aphis glycines-resistant plants produced by such selection and breeding methods are also provided herein.

Also provided herein are kits for selecting at least one soybean plant by marker-assisted selection of a quantitative trait locus associated with resistance to Aphis glycines comprising: (a) primers or probes for detecting at least one Aphis glycines resistance-associated marker locus, such as primers selected from the group consisting of primers for ss107918249, 27A SNP456169.7, KIM5. SNP65906.2, 56B. KIM3, 21A, 25A, 83A, SNP7623, ss107913360, SNP442-1688, and SNP86377; and (b) instructions for using the primers or probes for detecting the marker loci and correlating the loci with predicted Aphis Glycines resistance. In embodiments, the kits can include probes for detecting the marker loci and correlating the loci with predicted Aphis glycines resistance, packaging materials for packaging the probes, primers or instructions, controls such as control amplification reactions that include probes, primers or template nucleic acids for amplifications, molecular size markers, and other components known to the art for performing marker-assisted selection in plants.

Also provided herein is a method for producing an Aphis glycines-resistant soybean crop in a field comprising planting the field with crop seeds or plants that are resistant to Aphis glycines as a result of said seeds or plants containing one or more Aphis glycines resistance alleles in one or more marker locus localizing within a chromosomal interval on chromosome 7 associated with markers ss107918249, 27A, SNP456169.7, KIM5, NP65906.2, 56B, KIM3, 21A, 25A, 83A, SNP7623, ss107913360, SNP442-1688, and SNP86377, and markers mapping within 5 cM of said markers. In embodiments, the marker locus is selected from the group consisting of loci associated with markers SNP456169.7, KIM5, SNP65906.2, 56B, and KIM3 and markers mapping within 5 cM of said markers.

Further provided herein is a method for developing further markers useful for the above methods, comprising producing one or more primers for markers associated with Rag1 Aphis glycines resistance. The method comprises: (a) providing a first nucleotide sequence of soybean chromosome 7 from a soybean plant having Rag1 resistance to Aphis glycines wherein said first nucleotide sequence maps to a DNA interval flanked on one side by marker SNP46169.7 or KIM5 or markers within 5 cM of said markers, and flanked on the other side by KIM3 or markers within 5 cM of KIM3; and (b) providing a second nucleotide sequence corresponding to the first sequence from a plant known to lack Rag1 Aphis glycines resistance; (c) selecting at least one forward and reverse marker primer pair with oligonucleotide lengths between about 10 or 15 and about or 75 base pairs from the first nucleotide sequence; (d) separately amplifying genomic DNA from the primers in media containing DNA from the susceptible and resistant plants, respectively, to form amplification products; (e) selecting amplification products which are the only amplification products produced by said primers in each medium, i.e., where the primers produce only a single amplification product; (f) determining the presence of polymorphisms between the selected amplification products from the susceptible and resistant soybean DNA; and (g) selecting primers that produce polymorphic amplification products as primers for markers associated with Rag1 Aphis glycines resistance. The presence of polymorphisms can be determined by direct sequencing or by melt-curve analysis or other means known to the art. The amplification products are typically between about 200 and 1000 kb in length. The plant known to lack Aphis glycines resistance can be a soybean plant or other plant known to lack Aphis glycines resistance and to be subject to aphid attack, typically a legume. The method can also comprise selecting a probe having a sequence from a DNA interval between said forward and reverse primer sequences containing a polymorphism.

Rag1 aphid (Aphis glycines) resistance can be conferred on a plant by nucleic acid sequences described herein. Plants that do not contain these sequences can be transformed to contain them, or the sequences can be transferred to plants that lack them by crossbreeding, all by techniques known to the art. Aphid resistance can be tested by means known by the art, and as described hereinbelow.

A nucleotide molecule provided herein can be operably linked upstream at the 5′ end to a promoter capable of causing expression of said molecule in a host plant. Sequences of the native promoters are contained within SEQ ID NO:17 and these can be used to drive expression in appropriate tissues, as can be ascertained by one skilled in the art without undue experimentation.

Provided are bacteria, recombinant cells, and other vectors known to the art comprising the nucleic acid molecules disclosed herein. Specifically, provided herein are recombinant host cells comprising the nucleic acid sequences described above, which sequences are non-native to their host cells. In embodiments, these recombinant host cells are transgenic plant cells and plants containing these transgenic plant cells. Also provided are bacteria containing constructs expressing the proteins encoded by the sequences. Additionally, plants that are created by means of legitimate or illegitimate genetic recombination with the germplasm containing the above nucleic acid sequences, without the use of transgenic technology, are provided.

Transgenic plant cells comprising the nucleic acid molecules described above and transgenic plants and their progeny comprising such cells are also provided.

The transgenic plant can be one which, prior to being transformed with the nucleic acid molecules hereof, was a plait susceptible to aphid infestation. Such plants can include those selected from the group consisting of legumes (e.g., selected from the group consisting of soybean, alfalfa, clover, pea, bean, lentil, lupin, mesquite, carob, and peanut), apple, apricot, pear, plum, blackberry, blueberry, strawberry, cranberry, lemon, maize, wheat, rye, barley, oat, buckwheat, sorghum, rice, sunflower, canola, pea, bean, cotton, linseed, cauliflower, asparagus, lettuce, tobacco, mustard, sugarbeet, potato, sweet potato, carrot, turnip, celery, tomato, eggplant, cucumber, and squash. Monocot and dicot plants to which the Rag1 sequences and techniques described herein can be applied include agronomic and horticultural crop plants. Examples of other agronomic crop plants in which aphid-resistance can be created by transformation with the Rag1 sequences described herein include cereals such as maize, wheat, rye, barley, oats, buckwheat, sorghum and rice; non-cereals such as sunflower, canola, cotton and linseed; vegetables such cauliflower, asparagus, lettuce, tobacco and mustard; and root crops such as sugarbeet, potato, sweet potato, carrot and turnip, as well as horticultural crops such celery, and tomato, and fruit crops including apple, apricot, peach, pear, plum, orange, blackberry, blueberry, strawberry, cranberry and lemon.

A method for preparing such a transgenic plant is also provided herein, comprising: (a) selecting a host plant cell; (b) transforming the host plant cell with a construct comprising a nucleotide molecule as described above; (c) obtaining a transformed plant cell; and (d) regenerating a transgenic plant from the transformed plant cell, wherein the transgenic plant demonstrates increased aphid resistance relative to a non-transgenic plant of the same species.

The host plant cell can be a cell from a plant that contains no aphid resistance, or can be a cell from a plant that already contains aphid resistance, which can be Rag1 or other aphid resistance, such as Rag2 aphid resistance. The transformed plants claimed herein have more aphid resistance than untransformed plants of the same variety or species.

A method for identifying the presence or absence of a gene coding for resistance to Aphis glycines in soybean germplasm is also provided. In an embodiment, a method for determining the presence or absence of a gene for Rag1 resistance to Aphis glycines in soybean germplasm on soybean chromosome 7 (formerly Linkage Group M), is provided. The method comprises: analyzing said germplasm by marker-assisted selection (MAS) to: genomic DNA from a soybean germplasm for the presence of at least one molecular marker, wherein the at least one molecular marker is linked to the Rag1 gene locus to: detect a resistance to Aphis glycines (Rag1) locus that maps to soybean chromosome 7 of said soybean germplasm, wherein said Rag1 locus is flanked on opposite sides by markers KIM3 and markers mapping within 5 cM thereof, and SNP46169.7 and/or KIM5 and markers mapping within 5 cM thereof, which show allelic polymorphism between Aphis glycines-resistant and Aphis glycines-susceptible soybean genotypes and are linked to the Rag1 locus, and wherein the Rag1 locus comprises allelic DNA sequences that control resistance to Aphis glycines; and determine the presence or absence of an allelic form of DNA linked to the Rag1 gene coding for resistance to Aphis glycines in said germplasm; wherein the presence or absence of said allelic form of DNA linked to said gene is determined by comparing a first PCR-amplified polymorphic marker fragment of said soybean germplasm to a second PCR-amplified polymorphic marker fragment of soybean germplasm from a plant having Aphis glycines resistance conferred by said Rag1 gene, wherein said second fragment is made using the same marker that was used to make said first fragment, and wherein said second fragment has a size substantially the same as that of a PCR-amplified polymorphic marker fragment of germplasm of Aphis glycines-resistant soybean variety Dowling (PI 548663) made using the same marker used to make said first and second fragments; and determining that said gene coding for Rag1 resistance is present in said soybean germplasm when said first fragment is substantially the same size as said second fragment, and determining that said gene is not present in said germplasm when said first fragment is not substantially the same size as said second fragment.

The method can be performed by selecting two or more molecular markers, and in embodiments two markers are selected that flank the Rag1 gene on opposite sides. One of the flanking markers can be selected from the group consisting of ss107918249, 27A, SNP46169.7, KIM5, SNP65906.2, and 56B, and the other flanking marker can be selected from the group consisting of KIM3, 21A, 25A, 83A, SNP7623, ss107913360, SNP442-1688, and SNP86377. In embodiments the flanking markers are (1) KIM3 and (2) SNP46169.7 or KIM5. Markers derived from SEQ ID NO:17 as described in Paragraphs [0014]-[0016] can also be employed for this purpose.

Also provided herein is a method for reliably and predictably introgressing soybean Aphis glycines resistance conferred by the Rag1 gene from a first plant comprising the Rag1 gene into a second plant not comprising the Rag1 gene or comprising fewer copies of the Rag1 gene than the first plant. The method comprises: (a) providing a first soybean germplasm that comprising the Rag1 gene; (b) providing said second soybean germplasm; (c) crossing the first soybean germplasm with the second soybean germplasm to provide progeny soybean germplasm; (d) analyzing said progeny germplasm to determine the presence of the Rag1 gene by the MAS methods described herein; and (e) selecting progeny that tests positive for the presence of the Rag1 gene as being soybean germplasm into which Aphis glycines resistance conferred by the Rag1 gene has been introgressed.

### BRIEF DESCRIPTION OF THE FIGURES

FIG. 1 is a diagram showing rearrangement of the Rag1 aphid resistance interval between the susceptible cultivar Williams (top) and the aphid-resistant cultivar Dowling (bottom). “LRR” indicates predicted leucine-rich repeat genes. The amino acid sequences of the numbered intervals in Dowling are set forth in SEQ ID NOS:1-16. The nucleotide sequence of the entire interval in Dowling is set forth in SEQ ID NO:17.

FIG. 2 is a genetic linkage map for the interval between Satt463 and Satt540 on soybean chromosome 7 [formerly linkage group (LG) M] based on 824 BC₄F₂ plants.

FIG. 3 is a diagram of the procedure for Zoom In PCR (ZIP) fosmid library screening used herein.

FIG. 4 shows alignment of a region of the sequences of PCR products derived from the bands derived from ZIP screening compared with the sequences of the Dowling and Williams 82 genomic DNA control bands.

### DETAILED DESCRIPTION

Disclosed herein is an isolated nucleic acid molecule from soybean (Glycine max) [SEQ ID NO:17] comprising an aphid resistance gene. Isolated exons from this sequence providing aphid resistance are also disclosed. These nucleic acid sequences can be transformed into plants for conferring aphid resistance or enhanced aphid resistance on the transformed plants. Promoters and other sequences for regulating gene expression can be linked as known to the art to the aphid resistance sequences provided herein. Also disclosed are aphid-resistant soybean and other plants containing and expressing the nucleic acid sequences. Methods for conferring aphid resistance on soybean cultivars and other plants are also provided.

The Rag1 aphid (Aphis glycines) resistance can also be transferred to plants that lack them by crossbreeding, all by techniques known to the art. Aphid resistance can be tested by means known by the art, and as described hereinbelow.

Exogenous genetic material such as the Rag1 sequence disclosed herein, or portions thereof, or a mutated or chimeric Rag1 nucleic acid sequences can be transferred into a plant cell by use of a DNA vector or construct designed for such purpose. Design of such a vector is within the skill of the ordinary skilled artisan (see, Plant Molecular Biology: A Laboratory Manual, Clark eds, Springer, New York, 1997).

Also provided are bacteria, recombinant cells, and other vectors known to the art comprising the nucleic acid molecules disclosed herein. Specifically, provided herein are recombinant host cells comprising the nucleic acid sequences described above, which sequences are non-native to their host cells. The host plant cell can be a cell from a plant that contains no aphid resistance, or can be a cell from a plant that already contains aphid resistance, which can be Rag1 or other aphid resistance, such as Rag2 aphid resistance. The transformed plants claimed herein have more aphid resistance than untransformed plants of the same variety or species. In embodiments, the recombinant host cells are transgenic plant cells. Plants containing these transgenic plant cells are also provided, as are bacteria and other host cells containing constructs expressing the proteins encoded by the sequences. Additionally, plants that are created by means of legitimate or illegitimate genetic recombination with the germplasm containing the above nucleic acid sequences, without the use of transgenic technology, and selected by means described herein, are provided. Progeny of such recombinant cells and plants are also provided. A transgenic plant provided herein can be one which, prior to being transformed with the nucleic acid molecules hereof, was a plant susceptible to aphid infestation. A method for preparing such a transgenic plant is also provided herein, comprising: (a) selecting a host plant cell; (b) transforming the host plant cell with a construct comprising a nucleotide molecule as described above; (c) obtaining a transformed plant cell; and (d) regenerating a transgenic plant from the transformed plant cell, wherein the transgenic plant demonstrates increased aphid resistance relative to a non-transgenic plant of the same species.

Methods and compositions for transforming bacteria and other microorganisms are known in the art (see for example Sambrook et al., Molecular Cloning: A Laboratory Manual, Second Edition, Cold Spring Harbor Laboratory Press, Cold Spring Harbor, N.Y., 1989).

The Rag1 nucleic acid molecules described herein can be transferred into a plant cell and the plant cell regenerated into a whole plant. The Rag1 nucleic acid molecules can be from any source, whether naturally occurring or otherwise, obtained through methodologies in the field that are known to those skilled in the art, and are capable of being inserted into any plant cells. The Rag1 nucleic acid molecules can be transferred into monocotyledonous or dicotyledonous plants (Chistou, Particle Bombardment for Genetic Engineering of Plants, Biotechnology Intelligence Unit, Academic Press, San Diego, Calif., 1996).

There are many methods for transforming the Rag1 nucleic acid molecules into plant cells such as soybean plant cells. Suitable methods are believed to include virtually any methods by which nucleic acid molecules can be introduced into the cells, such as by Agrobacterium infection or direct delivery of nucleic acid molecules that can include PEG-mediated transformation, electroporation and acceleration of DNA coated particles, etc. (Pottykus, Ann. Rev. Plant Physiol. Plant Mol. Biol. 42:205 225, 1991; Vasil, Plant Mol. Biol. 25: 925 937, 1994). Five commonly used general methods for delivering a gene into cells are: (1) chemical methods (Graham and van der Eb, Virology, 54:536 539, 1973); (2) physical methods such as microinjection (Capecchi, Cell 22:479 488, 1980), electroporation (Wong and Neumann, Biochem. Biophys. Res. Commun. 107:584 587, 1982; Fromm et al., Proc. Natl. Acad. Sci. (USA) 82:5824 5828, 1985) and the gene gun (Johnston and Tang, Methods Cell Biol. 43:353 365, 1994); (3) viral vectors (Clapp, Clin. Perinatol. 20:155 168, 1993; Lu et al., J. Exp. Med. 178:2089 2096, 1993; Eglitis and Anderson, Biotechniques 6:608 614, 1988); (4) receptor-mediated mechanisms (Curiel et al., Hum. Gen. Ther. 3: 147 154, 1992; Wagner et al., Proc. Natl. Acad. Sci. (USA) 89: 6099 6103, 1992); and (5) Agrobacterium-mediated transformation (agrotransformation) using the binary or Ti plasmid to transfer T-DNA sequences to the plant genome as is known to the art.

Transformation of plant protoplasts can be achieved using methods based on calcium phosphate precipitation, polyethylene glycol treatment, electroporation, and combinations of these treatments (see for example (Potrykus et al., Mol. Gen. Genet. 205:193 200, 1986; Lorz et al., Mol. Gen. Genet. 199:178, 1985; Fromm et al., Nature 319:791, 1986; Uchimiya et al., Mol. Gen. Genet. 204: 204, 1986; Callis et al., Genes and Development 1183, 1987; Marcotte et al., Nature 335:454, 1988). Application of these systems to different plant strains depends upon the ability to regenerate that particular plant strain from protoplasts. For example, illustrative methods for the regeneration of cereals from protoplasts are known to the art. (Fujimura et al., Plant Tissue Culture Letters, 2:74, 1985; Toriyama et al., Theor. Appl. Genet. 205:34, 1986; Yamada et al., Plant Cell Rep. 4: 85, 1986; Abdullah et al., Biotechnology 4:1087, 1986).

In an embodiment hereof, transformation can be implemented by using a particle gun as described above (Johnston and Tang, Methods Cell Biol. 43:353 365, 1994). The Rag1 nucleic acid molecules can be coated on particles and projected into plants tissues ballistically. In another embodiment, Agrobacterium-mediated transformation technology can be used to introduce the Rag1 nucleic acid molecule into the soybean plant to achieve a desired result. Agrobacterium-mediated transfer is a widely applicable system for introducing genes, such as the Rag1 gene, into plant cells. The use of Agrobacterium-mediated plant integrating vectors to introduce a nucleic acid into plant cells is well known in the art. See, for example, Fraley et al. (Biotechnology 3:629 635, 1985), Hiei et al. (U.S. Pat. No. 5,591,616), and Rogers et al. (Meth. In: Enzymol. 153: 253 277, 1987).

In transformation methods described herein, polynucleotide inserts can be operatively linked to an appropriate promoter, such as the phage lambda PL promoter, the E. coli lac, trp, phoA and tac promoters, and the plant-expressible promoters disclosed herein and/or known to the art. In addition, promoters for this purpose can be selected from other organisms as known to the art, and include promoters selected from the group consisting of the promoters of other known plant disease and herbivore resistance genes, and promoters of the other genes in the genome of soybeans, such as soybean cv. Williams 82, which is almost completely sequenced (Schmutz, J., et al. (Jan. 14, 2010), “Genome sequence of the palaeopolyploid soybean,” Nature 463:178-183), and known promoters often used for the expression of transgenic constructs in plants, such as those of plant ubiquitin genes, promoters of transcription factors, cellulases, polygalacturonases, nopaline synthase (NOS), octopine synthase (OCS), mannopine synthase (mas), cauliflower mosaic virus 19S and 35S (CaMV19S, CaMV35S), enhanced CaMV (eCaMV), ribulose 1,5-bisphosphate carboxylase (ssRUBISCO), figwort mosaic virus (FMV), CaMV derived AS4, tobacco RB7, wheat POX1, tobacco EIF-4, lectin protein (LeI), and rice RC2 promoter. When desired, promoters or other elements that upregulate or downregulate expression can be used, such as the CaMV 35S promoter that upregulates expression, the Nopaline Synthase terminal which downregulates expression. Promoters that are known to or are found to cause transcription of DNA as mentioned above can be used for DNA transcription in target tissues or cell types. Such promoters can be obtained from a variety of sources such as plants and plant viruses. The particular promoter selected should be capable of causing sufficient expression to result in the production of an effective amount of polypeptide to cause the desired phenotype. In addition to promoters which are known to cause transcription of DNA in plant cells, other promoters can be identified for use in the present processes by screening a plant cDNA library for nucleic acids which are selectively or preferably expressed in target tissues or cells. For example, for the purpose of expression in source tissues of the plant, such as the leaf, seed, root or stem, it is preferred that the promoters utilized have relatively high expression in these specific tissues. Similarly, for the purpose of expression of a DNA of interest in sink tissues of the plant, such as the tuber of the potato plant, the fruit of tomato, or the seed of maize, wheat, rice, and barley, it is preferred that the promoters utilized have relatively high expression in these specific tissues. These promoters can be tissue-specific or show enhanced expression in these tissues. The expression constructs can further contain sites for transcription initiation, termination, and, in the transcribed region, a ribosome binding site for translation. The coding portion of the transcripts expressed by the constructs can include a translation initiating codon at the beginning and a termination codon (UAA, UGA or UAG) appropriately positioned at the end of the sequence to be translated. A 5′ untranslated sequence can also be employed adjacent to the end of the coding sequence. The 5′ untranslated sequence is the portion of an mRNA which extends from the 5′ CAP site to the translation initiation codon. This region of the mRNA is necessary for translation initiation in plants and plays a role in the regulation of gene expression. Suitable 5′ untranslated regions for use in plants include those of alfalfa mosaic virus, cucumber mosaic virus coat protein gene, and tobacco mosaic virus, among others.

Host plant cells transformed to contain the nucleic acids described herein, are regenerated to whole, fertile plants comprising the aphid-resistance trait. The regenerated plants, such as the regenerated soybean plants that contain the Rag1 nucleic acids described herein, which can be wild type, modified, or chemically synthesized, that encode the Rag1 proteins, can be self-pollinated to provide homozygous transgenic soybean plants. Otherwise, pollen obtained from the regenerated soybean plants can be crossed to seed-grown plants of agronomically important lines. Pollen from plants of these important lines can also be used to pollinate regenerated plants. A transgenic soybean plant of the present invention can be cultivated using methods well known to one skilled in the art.

A transgenic plant such as a transgenic soybean plant formed using the above-mentioned transformation methods can contain a single added Rag1 gene on one chromosome. Such a transgenic plant can be referred to as being heterozygous for the added Rag1 gene. In addition, a transgenic plant that is homozygous for the added Rag1 gene: i.e. a transgenic plant that contains two added Rag1 genes, one gene at the same locus on each chromosome of a chromosome pair can be produced. A homozygous transgenic plant can be obtained by sexually mating (selfing) an independent segregated transgenic plant that contains a single added Rag1 gene, germinating some of the seeds produced and analyzing the resulting plants produced for the Rag1 gene.

It is understood that two different transgenic plants can also be mated to produce offspring that contain two independently-segregating added, exogenous Rag1 genes. Setting of appropriate progeny can produce plants that are homozygous for both added exogenous Rag1 genes that encode Rag1 polypeptides. Back-crossing to a parental plant and out-crossing with a non-transgenic plant are also useful techniques, as is vegetative propagation.

A number of sequencing techniques are known in the art, including fluorescence-based sequencing methodologies. These methods have the detection, automation and instrumentation capability necessary for the analysis of large volumes of sequence data. The sequences in this patent were determined either with the Roche 454 FLX Titanium pyrosequencer or the ABI 3730 capillary sequencer. With these types of automated systems, either fluorescent dye-labeled sequence reaction products are detected and data entered directly into the computer, producing a chromatogram, or luminescent data revealing nucleotide incorporation when nucleotides are “flowed” is detected with a photomultiplier and computationally processed to produce a flowgram, which are subsequently viewed, stored, and analyzed using software programs. These methods are known to those of skill in the art.

Also provided herein are computer systems comprising a processor and a data storage device wherein the data storage device has stored thereon a polypeptide sequence or a nucleic acid sequence described herein (e.g., a polypeptide encoded by a nucleic acid molecule provided herein that confers or contributes to conferring aphid resistance to a plant). In one aspect, the computer system can further comprise a sequence comparison algorithm and a data storage device having at least one reference sequence stored thereon. In another aspect, the sequence comparison algorithm comprises a computer program that indicates polymorphisms. In one aspect, the computer system can further comprise means for identifying one or more features in said sequence. Provided herein are computer-readable media having stored thereon a polypeptide sequence or a nucleic acid sequence described herein. The step of determining differences between a first sequence that confers aphid resistance to a plant and a second sequence that is not known to confer aphid resistance to a plant can comprise the step of operating the computer program to identify polymorphisms between the sequences. In an aspect, the method can comprise reading a first sequence using a computer program and identifying one or more features in the sequence.

Also provided herein is a method for reliably and predictably introgressing soybean Aphis glycines resistance conferred by the Rag1 gene from a first plant comprising the Rag1 gene into a second plant not comprising the Rag1 gene or comprising fewer copies of the Rag1 gene, the method comprising: (a) providing a first soybean germplasm that comprising the Rag1 gene; (b) providing said second soybean germplasm; (c) crossing the first soybean germplasm with the second soybean germplasm to provide progeny soybean germplasm; (d) analyzing said progeny germplasm to determine the presence of the Rag1 gene; and (e) selecting progeny that tests positive for the presence of the Rag1 gene as being soybean germplasm into which Aphis glycines resistance conferred by the Rag1 gene has been introgressed by analyzing genomic DNA from the germplasm by the above-described method.

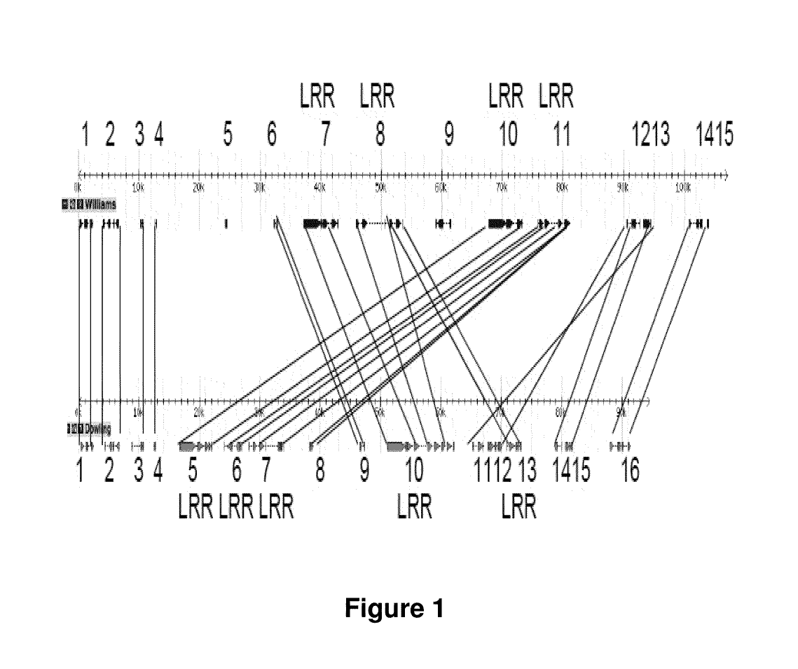

The Rag1 locus is known to the art to be flanked on opposite sides by Satt435 and Satt463 (U.S. Patent Publication No. 20060015964) and has now been further delimited using fine mapping of more markers, and found to be flanked on opposite sides by the genetic single nucleotide polymorphism (SNP) markers 46169.7 and KIM on one side, and KIM3 on the other side. These markers were designed based on the genome sequence of Williams 82 (an aphid-susceptible cultivar) (Schmutz, J., et al. (Jan. 14, 2010), “Genome sequence of the paleopolyploid soybean,” Nature 463: 178-183). The markers are located respectively at the physical nucleotide positions 5,608,084 and 5,508,533 on Williams 82 Chromosome 7, and are thus separated by a physical distance of 99,551 base pairs. Using the physical map and sequence of this region, PCR markers were designed such that they could be used to identify and retrieve fosmid clones containing orthologous DNA to this region from genomic libraries created using genomic DNA from the resistant cultivar, Dowling. Zoom-In PCR was used to identify four overlapping positive fosmid clones covering the orthologous region of this interval in the Dowling cultivar. By mass-sequencing of the aforesaid four clones using the 454 sequencing technology, we have created and assembled the complete sequence of this interval in the Dowling cultivar [SEQ ID NO:17]. Any errors in this sequence are believed to constitute less than 1%. Sequences having at least 95% or at least 97% or at least 98% or at least 99% homology to SEQ ID NO:17 are considered equivalent to this sequence. When the genes within this sequence were automatically detected and compared to the automated annotation of the Williams 82 genome sequence, extensive rearrangement within the cluster of Leucine-Rich Repea: (LRR) genes was visible (FIG. 1). The rearrangement of this cluster of LRR genes is the source of the aphid-resistance phenotype. Aphid resistance genes from other species have also been identified, and many belong to the LRR gene family.

We have thus identified a region of the genomic DNA of the soybean cultivar Dowling containing the Rag1 gene for aphid resistance, whereby plants that possess this DNA are resistant to soybean aphid infestation. The DNA contains a small cluster of leucine-rich-repeat genes that confer aphid resistance in the soybean line from which this DNA was derived. We have identified the precise sequence of the Dowling genome that can convert aphid-sensitive soybean genotypes (such as most of those currently grown commercially) into aphid-resistant soybean genotypes. This DNA sequence is used (1) to create soybean lines where introgression (cross-breeding) is conducted to precisely move this piece of DNA from an aphid-resistant line to an aphid-sensitive line, (2) to generate molecular markers in order to facilitate introgression of this locus, (3) to access aphid-resistant germplasm that can carry Rag1 to determine whether it has this gene, and/or 4) to develop transgenic plants where this piece of DNA, or part of it, containing a gene for aphid resistance is transferred using transgenic technology such as Agrobacterium, particle bombardment or other ways known to the art of artificially introducing genes into plants. This DNA sequence is also used to identify or create variants in the Rag1 gene that are capable of overcoming resistant aphid populations, and in the identification of other aphid-resistance genes in the genomes of soybean and other plants using sequence similarity searches.

The aphid-resistance gene sequence or a subsequence thereof or a sequence complementary to such sequence or subsequence can be fully or partially chemically synthesized by means known to the art, e.g., as described in Maniatis et al., Molecular Cloning: A Laboratory Manual, Cold Spring Harbor, N.Y. (1982), Sambrook et al., Molecular Cloning: A Laboratory Manual, Cold Spring Harbor, N.Y. (1989); or Ausubel 1993, Current Protocols in Molecular Biology, Wiley, NY. DNA sequences can be synthesized by phosphoramidite chemistry in an automated DNA synthesizer. In addition, a sequence of the aphid sensitive coding sequence can be modified, for example, by site directed mutagenesis techniques such as mutagenic polymerase chain reaction, or by transformation with a mutagenic oligonucleotide to achieve the desired result.

The DNA constructs of the invention can be used to transform any type of plant cells. DNA segments encoding a specific gene can be introduced into recombinant host cells and employed for expressing a specific structural or regulatory protein. Alternatively, through the application of genetic engineering techniques, subportions or derivatives of selected genes can be employed. Upstream regions containing regulatory regions such as promoter regions can be isolated and subsequently employed for expression of the selected gene.

Where an expression product is to be generated, the nucleic acid sequences can be varied so long as they retain the ability to encode the same product. Reference to the known codon preferences permit those of skill in the art to design any nucleic acid encoding for the product of a given nucleic acid in a desired host.

A genetic marker can be used for selecting transformed plant cells (“a selection marker”). Selection markers typically allow transformed cells to be recovered by negative selection (i.e., inhibiting growth of cells that do not contain the selection marker) or by screening for a product encoded by the selection marker. In certain embodiments, DNA fragments can be introduced into the cells of interest by the use of a vector, so as to bring the incorporation into the genome, replication and/or expression to the attached segment. A vector can have one or more restriction endonuclease recognition sites at which the DNA sequences can be cut in a determinable fashion without loss of an essential biological function of the vector. Vectors can further provide primer sites (e.g. for PCR), transcriptional and/or translational initiation and/or regulation sites, recombinational signals, replicons, selectable markers, etc. Examples of vectors include plasmids, phages, cosmids, phagemid, yeast artificial chromosome (YAC), bacterial artificial chromosome (BAC), human artificial chromosome (HAC), virus, virus based vector, and other DNA sequences which are able to replicate or to be replicated in vitro or in a host cell, or to convey a desired DNA segment to a des red location within a host cell. Polynucleotides can be joined to a vector containing a selectable marker for propagation in a host. If the vector is a virus, it can be packaged in vitro using an appropriate packaging cell line and then transduced into host cells.

As indicated, the expression vectors can include at least one selectable marker. Exemplary markers can include, but are not limited to, G418, glutamine synthase, herbicide resistance or neomycin resistance for eukaryotic cell culture, and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells: fungal cells, such as yeast cells (e.g., Saccharomyces cerevisiae or Pichia pastoris (ATCC Accession No. 201178)); insect cells such as Drosophila S2 and Spodoptera Sf9 cells, and plant cells. Appropriate culture media and conditions for the particular host cells are known in the art.

In embodiments hereof, various whole-genome methods can be used to analyze nucleic acids. The methods usually in involve the detection of hybridization of genetic segments to detect the presence and level of the segments in the sample. Microarrays can be used, either spotted or synthesized on a surface. Methods involving beads, microbeads, magnetic beads or fiber bundles can also be employed. Commercial whole-genome gene expression microarrays can be obtained from Applied Biosystems, Affymetrix, Agilent, GE Healthcare, and Illumina.

Procedures used to detect the presence of nucleic acids capable of hybridizing to the detectable probe include well known techniques such as Southern blotting, Northern blotting, dot blotting, colony hybridization, plaque hybridization, and PCR. In some applications, the nucleic acid capable of hybridizing to the labeled probe can be cloned into vectors such as expression vectors, sequencing vectors, or in vitro transcription vectors to facilitate the characterization and expression of the hybridizing nucleic acids in the sample. For example, such techniques can be used to isolate and clone sequences in a genomic library or cDNA library which are capable of hybridizing to the detectable probe as described herein. A commonly used selectable marker gene for plant transformation is neomycin phosphotransferase II (nptII) which, when placed under the control of plant expression control signals, confers resistance to kanamycin. Fraley et al., Proc. Natl. Acad. Sci. USA, 80:4803 (1983). Another selectable marker gene is the hygromycin phosphotransferase gene which confers resistance to the antibiotic hygromycin. Vanden Elzen et al., Plant Mol. Biol., 5:299 (1985). Additional selectable marker genes of bacterial origin that confer resistance to antibiotics include gentamycin acetyl transferase, streptomycin phosphotransferase, aminoglycoside-3′-adenyl transferase, and the bleomycin resistance determinant (Hayford et al. 1988. Plant Physiol. 86:1216, Jones et al. 1987. Mol. Gen. Genet. 210:86; Svab et al. 1990. Plant Mol. Biol. 14:197, Hille et al. 1986. Plant Mol. Biol. 7:171). Other selectable marker genes confer resistance to herbicides such as glyphosate, glufosinate or bromoxynil (Comai et al. 1985. Nature 317:741-744, Stalker et al. 1988. Science 242:419-423, Hinchee et al. 1988. Bio/Technology 6:915-922, Stalker et al. 1988. J. Biol. Chem. 263:6310-6314, and Gordon-Kamm et al. 1990. Plant Cell 2:603-618). Other selectable markers useful for plant transformation include, without limitation, mouse dihydrofolate reductase, plant 5-enolpyruvylshikimate-3-phosphate synthase, and plant acetolactate synthase (Eichholtz et al. 1987. Somatic Cell Mol. Genet. 13:67, Shah et al. 1986. Science 233:478, Charest et al. 1990. Plant Cell Rep. 8:643; EP 154,204). Commonly used, reporters for screening presumptively transformed cells include but are not limited to β-glucuronidase (GUS), β-galactosidase, luciferase, and chloramphenicol acetyltransferase (Jefferson, R. A. 1987. Plant Mol. Biol. Rep. 5:387, Teeri et al. 1989. EMBO J. 8:343, Koncz et al. 1987. Proc. Natl. Acad. Sci. USA 84:131, De Block et al. 1984. EMBO J. 3:1681), green fluorescent protein (GFP) (Chalfie et al. 1994. Science 263:802, Haseloff et al. 1995. TIG 11:328-329 and PCT application WO 97/41228). Another approach to the identification of relatively rare transformation events has been use of a gene that encodes a dominant constitutive regulator of the Zea cans anthocyanin pigmentation pathway (Ludwig et al. 1990. Science 247:449).

For applications in which the nucleic acid segments of the present invention are incorporated into vectors, such as plasmids, these segments can be combined with other DNA sequences, such as promoters, polyadenylation signals, restriction enzyme sites, multiple cloning sites, other coding segments, and others known to the art, such that their overall length can vary considerably. It is contemplated that a nucleic acid fragment of almost any length can be employed, with the total length preferably being limited by the ease of preparation and use in the intended recombinant DNA protocol.

Plasmid preparations and replication means are well known in the art. See for example, U.S. Pat. Nos. 4,273,875 and 4,567,146 incorporated herein their entirety. Some embodiments of the present invention include providing a portion of genetic material of a target cell and inserting the portion of genetic material of a target cell into a plasmid for use as an internal control plasmid.

By knowing the nucleotide sequences of the Rag1 genetic material in a cell and in an internal control, specific primer sequences can be designed. In an embodiment, at least one primer of a primer pair used to amplify a portion of genomic material of a cell is in common with one of the primers of a primer pair used to amplify a portion of genetic material of an internal control such as an internal control plasmid. In an embodiment, a primer is about, but not limited to 5 to about 50 oligonucleotides long, or about 10 to 40 oligonucleotides long or about 10 to about 30 oligonucleotides long. A number of template-dependent processes are available to amplify the marker sequences present in a given template sample. One of the best known amplification methods is the polymerase chain reaction (referred to as PCR) which is described in detail in U.S. Pat. Nos. 4,683,195, 4,683,202 and 4,800,159, and in Innis et al. 1990, each of which is incorporated herein by reference in its entirety. Suitable primer sequences for amplification can be readily synthesized by one skilled in the art or are readily available from third party providers such as BRL (New England Biolabs), and other suppliers known to the art. Other reagents, such as DNA polymerases and nucleotides, that are necessary for a nucleic acid sequence amplification such as PCR are also commercially available. Nucleic acids used as a template for amplification can be isolated from cells contained in a biological sample, according to standard methodologies. The nucleic acid can be genomic DNA or fractionated or whole cell RNA. Where RNA is used, it can be desired to convert the RNA to a complementary cDNA. In one embodiment, the RNA is whole cell RNA and is used directly as the template for amplification.

Pairs of primers that selectively hybridize to nucleic acids corresponding to specific markers are contacted with the isolated nucleic acid under conditions that permit selective hybridization. Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced. Next, the amplification product is detected. In certain applications, the detection can be performed by visual means. Alternatively, the detection can involve indirect identification of the product via chemiluminescence, radioactive scintilography of incorporated radiolabel or fluorescent label or even via a system using electrical or thermal impulse signals (Affymax technology; Bellus, 1994).

A reverse transcriptase PCR amplification procedure can be performed in order to quantify the amount of mRNA amplified. Methods of reverse transcribing RNA into cDNA are well known. Alternative methods for reverse transcription utilize thermostable DNA polymerases. These methods are described in WO 90/07641 filed Dec. 21, 1990. Polymerase chain reaction methodologies are well known in the art. Other amplification methods are known in the art besides PCR such as LCR (ligase chain reaction), disclosed in European Application No. 320 308, incorporated herein by reference in its entirety.

An isothermal amplification method, in which restriction endonucleases and ligases are used to achieve the amplification of target molecules that contain nucleotide 5′-[alpha-thio]-triphosphates in one strand of a restriction site can also be useful in the amplification of nucleic acids herein. Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA which is present in a sample. Upon hybridization, the reaction is treated with RNase H, and the products of the probe identified as distinctive products which are released after digestion. Tire original template is annealed to another cycling probe and the reaction is repeated. Still other amplification methods known in the art can be used with the methods described herein.

Following amplification, it can be desirable to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989. Alternatively, chromatographic techniques can be employed to effect separation of amplified product or other molecules. There are many kinds of chromatography which can be used: adsorption, partition, ion-exchange and molecular sieve, and many specialized techniques for using them including column, paper, thin-layer and gas chromatography.

Amplification products must be visualized (detected) in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation. Probes can be labeled with radioactive, fluorescent or other labels known to the art. In an embodiment hereof, the described methods use a fluorescence resonance energy transfer (FRET) labeled probe as an internal hybridization probe. In an embodiment, an internal hybridization probe is included in the PCR reaction mixture so that product detection occurs as the PCR amplification product is formed, thereby reducing post-PCR processing time. Roche Lightcycler PCR instrument (U.S. Pat. No. 6,174,670) or other real-time PCR instruments can be used in this embodiment of the present invention, e.g., see U.S. Pat. No. 6,814,934. PCR amplification of a genetic material increases the sensitivity. In some instances, real-time PCR amplification and detection significantly reduce the total assay time so that test results can be obtained in about 12 hours. Accordingly, methods herein provide rapid and/or highly accurate results relative to the conventional methods and these results are verified by an internal control. In embodiments involving hybridization, one can employ nucleic acid sequences or fragments or complements thereof as disclosed herein in combination with a detectable signal, such as a label, for determining hybridization. A wide variety of appropriate detectable agents are known in the art, including fluorescent, radioactive, enzymatic or other ligands, such as avidin/biotin, which are capable of being detected. One can employ a fluorescent label or an enzyme tag such as urease, alkaline phosphatase or peroxidase, instead of radioactive or other environmentally undesirable reagents. In the case of enzyme tags, calorimetric indicator substrates are known which can be employed to allow detection visible to the human eye or spectrophotometrically, to identify specific hybridization with complementary nucleic acid-containing samples.

In an embodiment, visualization is achieved indirectly. Following separation of amplification products, a labeled, nucleic acid probe is brought into contact with the amplified marker sequence. The probe can be conjugated to a chromophore or can be radiolabeled. In another embodiment, the probe is conjugated to a binding partner, such as an antibody or biotin, where the other member of the binding pair carries a detectable moiety. In one embodiment, detection is by Southern blotting and hybridization with a labeled probe. The techniques involved in Southern blotting are well known to those of skill in the art and can be found in many standard books on molecular biological protocols.

Embodiments hereof include providing conditions that facilitate amplification of at least a portion of a target genetic material. However, it should be appreciated that the amplification conditions are not necessarily 100% specific. The embodiments include any method for amplifying at least a portion of a cell's genetic material (such as polymerase chain reaction (PCR), real-time PCR (RT-PCR), and NASBA (nucleic acid sequence based amplification)). In an embodiment, real time PCR (RT-PCR) is the method used for amplifying at least a portion of a cell's genetic material while simultaneously amplifying an internal control for verification of the outcome of the amplification of a cell's genetic material. Amplification of a genetic material, e.g., DNA, is well known in the art. Methods include providing conditions that would allow co-amplification of an internal control portion of a cell's genetic material and a portion of the cell's genetic material of a test sample, if the target sequence is present in the sample. In this manner, detection of the amplification products by a specific probe for each product of the internal control portion of a cell's genetic material and a portion of the cell's genetic material is indicative of the presence of the Rag1 sequence in the sample and that the conditions for the amplification are working. Thus, a negative result indicative of absence of a target Rag2 sequence can be confirmed. Typically, to verify the working conditions of PCR techniques, positive and negative external controls are performed in parallel reactions to the sample tubes to test the reaction conditions, for example using a control nucleic acid sequence for amplification. In some embodiments, an internal control can be used to determine if the conditions of the RT-PCR reaction is working in a specific tube for a specific target sample. Alternatively, in some embodiments, an internal control can be used to determine if the conditions of the RT-PCR reaction are working in a specific tube at a specific time for a sample. The presence or absence of PCR amplification product can be detected by any of the techniques known to one skilled in the art. In one particular embodiment, methods of the present invention include detecting the presence or absence of the PCR amplification product using a probe that hybridizes to a particular Rag1 sequence. By designing the PCR primer sequence and the probe nucleotide sequence to hybridize different portions of the Rag1 genetic material, one can increase the accuracy and/or sensitivity of the methods disclosed herein.

In general, it is envisioned that the hybridization probes described herein are useful both as reagents in solution hybridization, as in PCR, for detection of presence of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The selected conditions depend on the particular circumstances based on the particular criteria required (depending, for example, on the G+C content, type of target nucleic acid, source of nucleic acid, size of hybridization probe, and other conditions known to the art). Following washing of the hybridized surface to remove non-specifically bound probe molecules, hybridization is detected, or even quantified, by means of the label.

Methods disclosed herein are not limited to the particular probes disclosed and particularly are intended to encompass at least nucleic acid sequences that are hybridizable to the disclosed sequences or are functional sequence analogs of these sequences. For example, a partial sequence can be used to identify a structurally-related gene or the full length genomic or cDNA clone from which it is derived. Those of skill in the art are well aware of the methods for generating cDNA and genomic libraries which can be used as a target for the above-described probes.

Certain embodiments involve incorporating a label into a probe, primer and/or target nucleic acid to facilitate its detection by a detection unit. A number of different labels can be used, such as Raman tags, fluorophores, chromophores, radioisotopes, enzymatic tags, antibodies, chemiluminescent, electroluminescent, affinity labels, etc. One of skill in the art will recognize that these and other label moieties not mentioned herein can be used in the disclosed methods.

Fluorescent labels of use can include, but are not limited to, Alexa 350, Alexa 430, AMCA (7-amino-4-methylcoumarin-3-acetic acid), BODIPY (5,7-dimethyl-4-bora-3a,4a-diaza-s-indacene-3-propionic acid) 630/650, BODIPY 650/665, BODIPY-FL (fluorescein), BODIPY-R6G (6-carboxyrhodamine), BODIPY-TMR (tetramethylrhodamine), BODIPY-TRX (Texas Red-X), Cascade Blue, Cy2 (cyanine), Cy3, Cy5,6-FAM (5-carboxyfluorescein), Fluorescein, 6-JOE (2′7′-dimethoxy-4′5′-dichloro-6-carboxyfluorescein), Oregon Green 488, Oregon Green 500, Oregon Green 514, Pacific Blue, Rhodamine Green, Rhodamine Red, ROX (6-carboxy-X-rhodamine), TAMRA (N,N,N′,N′-tetramethyl-6-carboxyrhodamine), Tetramethylrhodamine, and Texas Red. Fluorescent or luminescent labels can be obtained from standard commercial sources, such as Molecular Probes (Eugene, Oreg.). Examples of enzymatic labels include urease, alkaline phosphatase or peroxidase. Calorimetric indicator substrates can be employed with such enzymes to provide a detection means visible to the human eye or spectrophotometrically. Radioisotopes of potential use include 14 carbon, 3 hydrogen, 125 iodine, 32 phosphorous, 33 phosphorous, and 35 sulphur.

As described herein, an aspect of the present disclosure concerns isolated nucleic acids and methods of use of isolated nucleic acids. In certain embodiments, the nucleic acid sequences disclosed herein have utility as hybridization probes or amplification primers. These nucleic acids can be used, for example, in diagnostic evaluation of plant tissue samples. In certain embodiments, these probes and primers consist of oligonucleotide fragments. Such fragments should be of sufficient length to provide specific hybridization to an RNA or DNA tissue sample. The sequences typically will be 10-20 nucleotides, but can be longer. Longer sequences, e.g., 40, 50, 100, 500 and even up to full length, can be used for certain embodiments.

Nucleic acid molecules having contiguous stretches of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 125, 150, 175, 200, 250, 300, 400, 500, 600, 750, 1000, 1500, 2000, 2500 or more nucleotides from a sequence selected from the nucleic acid sequences set forth herein are contemplated. Molecules that are complementary to the above-mentioned sequences and that bind to these sequences under high stringency conditions also are contemplated. These probes are useful in a variety of hybridization embodiments, such as Southern and Northern blotting.

The use of a hybridization probe of between about 14 and 100 nucleotides in length allows the formation of a duplex molecule that is both stable and selective. Molecules having complementary sequences over stretches greater than 20 bases in length are generally preferred in order to increase stability and selectivity of the hybrid and thereby improve the quality and degree of particular hybrid molecules obtained. One generally designs nucleic acid molecules having stretches of 20 to 30 nucleotides, or even longer where desired by directly synthesizing the fragment by chemical means or by introducing selected sequences into recombinant vectors for recombinant production.

Accordingly, the nucleotide sequences herein can be used for their ability to selectively form duplex molecules with complementary stretches of genes or RNAs or to provide primers for amplification of DNA or RNA from tissues. Depending on the application envisioned, one can desire to employ varying conditions of hybridization to achieve varying degrees of selectivity of probe towards target sequence

### DEFINITIONS

As used herein, the terms “recombinant polynucleotide” and “recombinant nucleic acid molecule,” are used interchangeably to refer to linear or circular, purified or isolated polynucleotides that have been artificially designed and which comprise at least two nucleotide sequences that are not found as contiguous nucleotide sequences in their initial natural environment. As noted above, this disclosure provides a recombinant nucleic acid construct or expression vector that facilitates the expression of the Rag1 nucleic acid sequence discussed herein in plants. As used herein, the term “nucleic acid construct” for “DNA construct”) refers to nucleic acid fragments assembled through genetic engineering techniques operatively linked in a functional manner to direct the expression of a nucleic acid sequence of interest, such as the Rag1 nucleic acid sequence discussed herein. The construct can also include additional sequence(s) or gene(s) of interest. As used herein, the terms “recombinant polynucleotide” and “recombinant nucleic acid molecule,” are used interchangeably to refer to linear or circular, purified or isolated polynucleotides that have been artificially designed and which comprise at least two nucleotide sequences that are not found as contiguous nucleotide sequences in their initial natural environment. In particular, these terms mean that the polynucleotide or cDNA is adjacent to “backbone” nucleic acid to which it is not adjacent in its natural environment. Additionally, to be “enriched” the cDNAs will represent 5% or more of the number of nucleic acid inserts in a population of nucleic acid backbone molecules. Backbone molecules according to the present invention include nucleic acids such as expression vectors, self-replicating nucleic acids, viruses, integrating nucleic acids, and other vectors or nucleic acids used to maintain or manipulate a nucleic acid insert of interest. Preferably, the enriched cDNAs represent 15% or more of the number of nucleic acid inserts in the population of recombinant backbone molecules. More preferably, the enriched cDNAs represent 50% or more of the number of nucleic acid inserts in the population of recombinant backbone molecules. In a highly preferred embodiment, the enriched cDNAs represent 90% or more (including any number between 90 and 100%, to the thousandth position, e.g., 99.5%) of the number of nucleic acid inserts in the population of recombinant backbone molecules.

As used herein, the term “polypeptide” or “protein,” used interchangeably herein, refers to a polymer composed of amino acids connected by peptide bonds, without regard to the length of the polymer; thus, peptides, oligopeptides, and proteins are included within the definition of polypeptide. This term also does not specify or exclude chemical or post-expression modifications of the polypeptides herein, although chemical or post-expression modifications of these polypeptides can be included excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications can be specified as individual species to be included or excluded from the present invention. The natural or other chemical modifications, such as those listed in examples above can occur anywhere in a polypeptide, including the peptide backbone, the amino acid side-chains and the amino or carboxyl termini. It will be appreciated that the same type of modification can be present in the same or varying degrees at several sites in a given polypeptide. Also, a given polypeptide can contain many types of modifications. Polypeptides can be branched, for example, as a result of ubiquitination, and they can be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. Also included within the definition are polypeptides which contain one or more analogs of an amino acid (including, for example, non-naturally occurring amino acids, amino acids which only occur naturally in an unrelated biological system, modified amino acids from mammalian systems, etc.), polypeptides with substituted linkages, as well as other modifications known in the art, both naturally occurring and non-naturally occurring. The term “polypeptide” or “protein” also applies to any amino acid polymers in which one or more amino acid residue is an artificial chemical analog of a corresponding naturally-occurring amino acid, as well as to any naturally-occurring amino acid polymers. The essential nature of such analogs of naturally-occurring amino acids is that, when incorporated into a protein, which protein is specifically reactive to antibodies elicited to the same protein but consisting entirely of naturally-occurring amino acids. It is well known in the art that proteins or polypeptides can undergo modification, including but not limited to, disulfide bond formation, gamma-carboxylation of glutamic acid residues, glycosylation, lipid attachment, phosphorylation, oligomerization, hydroxylation and ADP-ribosylation. Exemplary modifications are described in most basic texts, such as, for example, Proteins—Structure and Molecular Properties, 2nd ed. (Creighton, Freeman and Company, N.Y., 1993). Many detailed reviews are available on this subject, such as, for example, those provided by Wold (In: Post-translational Covalent Modification of Proteins, Johnson, Academic Press, N.Y., pp. 112, 1983), Seifter et al. (Meth. Enzymol. 182: 626, 1990) and Rattan et al. (Ann. N.Y. Acad. Sci. 663: 48 62, 1992). Modifications can occur anywhere in a polypeptide, including the peptide backbone, the amino acid side chains and the amino or carboxyl termini. In fact, blockage of the amino or carboxyl group in a polypeptide, or both, by a covalent modification, is common in naturally-occurring and synthetic polypeptides and such modifications can be present in polypeptides described herein as well. For instance, the amino terminal residue of polypeptides made in E. coli or other cells, prior to proteolytic processing, almost invariably will be N-formylmethionine. During post-translational modification of the polypeptide, a methionine residue at the NH₂ terminus can be deleted. Accordingly, the polypeptides and proteins described herein include both the methionine-containing and the methionine-less amino terminal variants. Thus, as used herein, the term “protein” or “polypeptide” includes any protein or polypeptide that is modified by any biological or non-biological process yet still binds to antibodies to the unmodified protein or polypeptide. The terms “amino acid” and “amino acids” refer to all naturally-occurring and synthetic amino acids and, unless otherwise limited, to known analogs of natural amino acids that can function in a similar manner as naturally-occurring amino acids.

The terms “complementary” or “complement thereof” are used herein to refer to the sequences of polynucleotides which is capable of forming Watson & Crick base pairing with another specified polynucleotide throughout the entirety of the complementary region. For the purpose of the present invention, a first polynucleotide is deemed to be complementary to a second polynucleotide when each base in the first polynucleotide is paired with its complementary base. Complementary bases are, generally, A and T (or A and U), or C and G. “Complement” is used herein as a synonym from “complementary polynucleotide”, “complementary nucleic acid” and “complementary nucleotide sequence”. These terms are applied to pairs of polynucleotides based solely upon their sequences and not any particular set of conditions under which the two polynucleotides would actually bind. Unless otherwise stated, all complementary polynucleotides are fully complementary on the whole length of the considered polynucleotide.

As used herein, the term “operably linked” refers to a linkage of polynucleotide elements in a functional relationship. A sequence which is “operably linked” to a regulatory sequence such as a promoter means that said regulatory element is in the correct location and orientation in relation to the nucleic acid to control RNA polymerase initiation and expression of the nucleic acid of interest. For instance, a promoter or enhancer is operably linked to a coding sequence if it affects the transcription of the coding sequence.

The terms “participating in conferring” and “contributing to conferring” with respect to polynucleotides and polypeptides that can confer or help confer Aphis glycines resistance on a plant mean that aphid resistance already present in the plant is enhanced, or that the sequences provided herein can cooperate with other sequences already present in the plant or inserted into the plant genome to confer or enhance aphid resistance in the plant, or can be triggered by environmental conditions in order to confer aphid resistance to the plant, such as by being linked to promoters that respond to such environmental conditions.

The term “primer” as used herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers can be provided in double-stranded or single-stranded form, typically single-stranded form.

The term “array” includes a device known to the art as a “microarray” or “biochip” or “chip” that comprises a plurality of target elements, each target element comprising a defined amount of one or more polypeptides (including antibodies) or nucleic acids immobilized onto a defined area of a substrate surface, for example as defined in U.S. Pat. No. 7,592,434, incorporated herein by reference to the extent not inconsistent herewith.

The terms “computer,” “computer program” and “processor” are used herein in their broadest general contexts and incorporate all such devices known to the art.

A “coding sequence of or a “sequence that encodes” a particular polypeptide or protein, is a nucleic acid sequence that is capable of being is transcribed and translated into the polypeptide or protein when placed under the control of appropriate regulatory sequences.

Sequence “homology,” “identity,” and “similarity” can be measured using sequence analysis software, known to the art, for example, as described in U.S. Pat. No. 7,592,434. Such software matches similar sequences by assigning degrees of homology to various deletions, substitutions and other modifications. The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated legion as measured using any number of sequence comparison algorithms or by manual alignment and visual inspection. For sequence comparison, one sequence can act as a reference sequence, e.g., a sequence described and claimed herein, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. A “comparison window”, as used herein, includes reference to a segment of any one of the numbers of contiguous residues. For example, in aspects hereof, contiguous residues ranging anywhere from 20 bp or amino acid residues to the full length of an exemplary polypeptide or nucleic acid sequence of the invention are compared to a reference sequence of the same number of contiguous positions after the two sequences are optimally aligned. If the reference sequence has the requisite sequence identity to an exemplary polypeptide or nucleic acid sequence described herein, e.g., 85%, 86%, 87%, 88%, 89%, 90%, 91%, 92%, 93%, 94%, 95%, 96%, 97%, 98%, 99%, or more sequence identity to a sequence hereof, that sequence is within the scope of the claims hereof. In alternative embodiments, subsequences ranging from about 20 bp to 600 bp, about 50 bp or amino acid residues to 200 bp or amino acid residues, and about 100 to 150 bp or amino acid residues are compared to a reference sequence of the same number of contiguous positions after the two sequences are optimally aligned. Methods of alignment of sequence for comparison are well known in the art.

As used herein, the term “sequence identity” refers to amino acid or nucleic acid sequences that, when compared using the local homology algorithm of Smith and Waterman in the BestFit program (Wisconsin Package Version 10.0, Genetics Computer Group (GCG), Madison, Wis., 1981), are exactly alike. “Sequence identity” between two nucleotide or protein sequences can be determined by using programs such as a BLAST program (Altschul et al., Nucleic Acids Res. 25:3389 3402; 1997) using the following parameters:
p Program Name=blastx, blastn or blastp
d Database=nr
e Expectation value (F)=10
F Filter query sequence (DUST with blastn, SEG with others=T
G Cost to open a gap=−1
E Cost to extend a gap=−1
X X dropoff value for gapped alignment (in bits)=blastn 30, tblastx 0, all others
q Penalty for a nucleotide mismatch (blastn only)=−3
r Reward for a nucleotide match (blastn only)=1
f Threshold for extending hits, defaultblastp 11, blastn 0, blastx 12, tblastn 13 tblastx 13-g Perform gapped alignment (not available with tblastx)=T-Q Query Genetic code to use=1-D DB Genetic code (for tblast[nx] only)=1
M Matrix=BLOSUM62-W Word size=blastn 11, megablast 28, all others 3-z Effective length of the database (use zero for the real size)=O-K Number of best hits from a region to keep. As is known to one of skill in the art, other default settings can be used.

As used herein, the term “sequence similarity” refers to amino acid sequences that, when compared using the local homology algorithm of Smith and Waterman in the BestFit program (Wisconsin Package Version 10.0, Genetics Computer Group (GCG), Madison, Wis., 1981), match when conservative amino acid substitutions are considered.

As used herein, a “coding sequence,” “structural nucleotide sequence” or “gene” or “structural gene” is a nucleotide sequence that is translated into a polypeptide, usually via mRNA, when placed under the control of appropriate regulatory sequences. The boundaries of the coding sequence are determined by a translation start codon at the 5′-terminus and a translation stop codon at the 3′-terminus. A coding sequence can include, but is not limited to, genomic DNA, cDNA, and recombinant nucleotide sequences.

The “complement” of a nucleic acid sequence as used herein is a nucleic acid sequence that forms a double-stranded structure with another nucleic acid fragment by following base-pairing rules (for DNA, A pairs with T and C with G; for RNA A pairs with U and C pairs with G). The complementary sequence to GTAC for example, is CATG.

As used herein, a “recombinant” DNA, structure, or organism is one that does not exist in nature, and is produced only by genetic engineering techniques, such as isolation of nucleic acid, transforming an organism with DNA that the organism does not naturally contain, or does not naturally contain in the location where it is placed, synthesizing nucleic acids, adding to or deleting nucleic acids from pre-existing nucleic acids, mutating nucleic acids in vivo or in vitro, or artificially producing the “recombinant” element by any means known to the art.

As used herein, “expression” refers to the transcription and stable accumulation of mRNA derived from nucleic acid molecules or regions, e.g., nucleic acid molecules or regions described herein. “Expression” can also refer to translation of mRNA into a polypeptide.

“Expression control sequences” are DNA sequences involved in any way in the control of transcription or translation. Suitable expression control sequences and methods of making and using them are well known in the art and can be used with plants transformed to contain the nucleic acid constructs disclosed herein. The expression control sequences must include a promoter. The promoter can be any DNA sequence which shows transcriptional activity in the chosen plant cells, plant parts, or plants. The promoter can be inducible or constitutive. It can be naturally-occurring, can be composed of portions of various naturally-occurring promoters, or can be partially or totally synthetic. Guidance for the design of promoters is provided by studies of promoter structure, such as that of Harley and Reynolds, Nucleic Acids Res., 15, 2343-61 (1987). Also, the location of the promoter relative to the transcription start can be optimized. Many suitable promoters for use in plants are well known in the art.

As used herein “operatively linked” with respect to specific coding DNA and expression-modifying or expression-controlling DNA elements refers to the linking of the specific coding DNA elements (including the order of the elements, the orientation of the elements, and the relative spacing of the various elements) to the modifying or controlling elements in such a manner that the expression of the specific coding DNA is modified by the expression-modifying or controlling elements. Methods of operatively linking expression control sequences to coding sequences are well known in the art.

As used herein, a “genotype” refers to the genetic constitution, Latent or expressed, of all the genes present in an individual organism such as a plant. As used herein, a “phenotype” of an organism such as a plant is any of one or more characteristics of a plant (e.g. male sterility, yield, quality improvements, etc.), as contrasted with the genotype. A change in genotype or phenotype can be transient or permanent.

As used herein, an “analog” of a first nucleotide sequence refers to a second nucleic acid sequence that is functionally the same as the first nucleotide sequence. For example, an “analog” of the Rag1 nucleic acid sequence [SEG ID NO:17] is a nucleotide sequence from a plant species that encodes a polypeptide that is functionally equivalent to the polypeptide expressed by the Rag1 nucleic acid sequence in conferring aphid resistance on a plant carrying it and that has substantial amino acid sequence identity or similarity to the Rag1 polypeptide from soybean, for example at least about or 95% or at least about or 97% or at least about or 98% or at least about or 99% sequence identity or similarity.

As used herein, “hybridization” with respect to nucleic acids refers to a strand of nucleic acid joining with a complementary strand via base pairing. Hybridization occurs when complementary sequences in the two nucleic acid strands bind to one another.

As used herein, an “isolated” nucleic acid molecule is one that is separate from or purified away from other nucleic acid sequences in the cell of the organism in which the nucleic acid naturally occurs, i.e., such as by conventional nucleic acid-purification methods. The term embraces naturally-occurring nucleic acid sequences, recombinant nucleic acid sequences and chemically synthesized nucleic acid sequences.

As used herein, the term “isolated polypeptide” refers to a polypeptide separate from other polypeptides that are naturally present in an organism or cell, e.g., produced by expression of an isolated nucleic acid molecule described herein or produced by chemical synthesis. The term can also refer to a polypeptide that has been sufficiently separated from other polypeptides or proteins with which it would naturally be associated, so as to exist in substantially pure form. Also as used herein, a “functionally equivalent fragment” of a larger polypeptide refers to a polypeptide that lacks at least one residue of the larger polypeptide, e.g., lacks at least one residue from an end of the larger polypeptide. Such a fragment retains a functional activity of the full-length polypeptide when expressed in a transgenic plant and/or possesses a characteristic functional domain or an immunological determinant characteristic of the native larger polypeptide. Immunological y active fragments typically have a minimum size of 7 or 17 or more amino acids, for example 10 amino acids. Useful Rag1 fragments are generally at least 10 amino acids in length.

As used herein, “combinations of” polypeptide fragments or “combinations of nucleotide sequences encoding combinations of polypeptide fragments” can refer to separate fragments or to single nucleotide or polypeptide molecules in which the component fragments are bonded together, either in the order in which they occur naturally in the Rag1 interval, or in any rearranged order that functions to produce aphid resistance in a transformed plant.

As used herein, the term “native” with respect to a nucleic acid sequence or polypeptide refers to a naturally-occurring (“wild type”) nucleic acid sequence or polypeptide.

As used herein, a “percentage of sequence identity” is determined by comparing two optimally aligned sequences over a comparison window, wherein the portion of the polynucleotide sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the window of comparison and multiplying the result by 100 to yield the percentage of sequence identity. The percentage of sequence identity can be determined by using programs such as a BLAST program (Altschul et al., Nucleic Acids Res. 25:3389 3402, 1997) using the default parameters.

As used herein, “plant” means plant cells, plant protoplast, plant cell or tissue culture from which soybean plants can be regenerated, plant calli, plant clumps and plant cells that are intact in plants or parts of plants, such as seeds, pods, flowers, cotyledons, leaves, stems, buds, roots, root tips and other suitable plant parts.

As used herein, a “polymorphism” is a change or difference between two related nucleic acids. A “nucleotide polymorphism” refers to a nucleotide which is different in one sequence when compared to a related sequence when the two nucleic acids are aligned for maximal correspondence. A “genetic nucleotide polymorphism” refers to a nucleotide which is different in one sequence when compared to a related sequence when the two nucleic acids are aligned for maximal correspondence, where the two nucleic acids are genetically related, i.e., homologous, for example, where the nucleic acids are isolated from different varieties of a soybean plant, or from different alleles of a single variety.

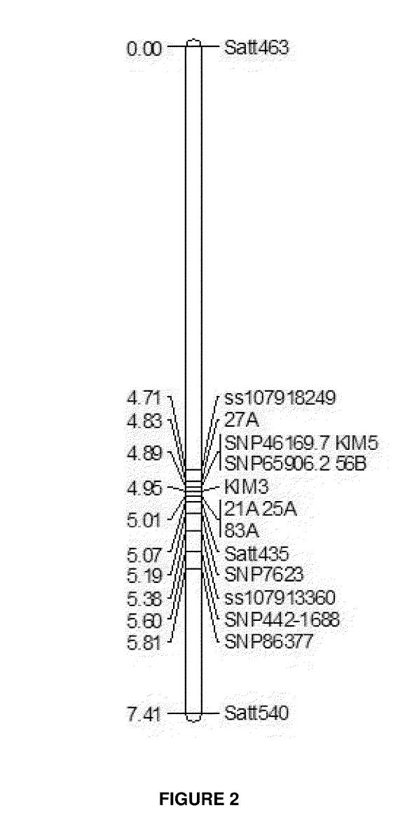

As used herein, “trait locus” refers to a chromosomal region where contains or is genetically linked (e.g., maps close to, such as within about 5 cM or about 10 cM of) a selected polymorphic nucleic acid or trait determinant. A “marker” for a particular trait is a DNA sequence that hybridizes to a trait locus. A “marker locus” is the location in DNA of an organism that hybridizes to an amplified DNA sequence, e.g., a PCR-amplified DNA sequence made from primers having a sequence in or mapping near the trait locus that contains a polymorphic trait determinant. Two loci or nucleic acid sequences on a chromosome are “genetically linked” when there is limited recombination between them during breeding. For example, if two loci are 5 cM apart on a linkage map such as shown in FIG. 2, there is a 5% chance that they will be separated by recombination during breeding. If they are 10 cM apart, there is a 10% chance they will be separated by recombination during breeding.

As used herein, a “promoter” refers to a DNA sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a DNA sequence that can stimulate promoter activity and can be an innate element of the promoter or a heterologous element inserted to enhance the expression level or tissue-specificity of a promoter. Promoters can be derived in their entirety from a native gene or be composed of different elements derived from different promoters found in nature, and/or can comprise synthetic DNA segments. It is understood by those skilled in the art that different promoters can direct the expression of a gene in different tissues or cell types, or at different stages of development, or in response to different environmental conditions. Promoters that cause a gene to be expressed in most cell types at most times are commonly referred to as “constitutive” promoters. Promoters that cause conditional expression of a structural nucleotide sequence under the influence of changing environmental conditions or developmental conditions are commonly referred to as “inducible promoters.”

As used herein, a “vector” is a composition which can transduce, transform or infect a cell, thereby causing the cell to express nucleic acids carried by the vector, and, optionally, proteins other than those native to the cell, or in a manner not native to the cell. A vector includes a nucleic acid (ordinarily RNA or DNA) to be expressed by the cell (a “vector nucleic acid”). A vector optionally includes materials to aid in achieving entry of the nucleic acid into the cell, such as a retroviral particle, liposome, protein coating or the like. The vector and/or other construct can also include, within the coding region of interest, a nucleic acid sequence that acts, in whole or in part, to terminate transcription of that region. For example, such termination sequences include the Tr7 3′ sequence and the nos 3′ sequence (Ingelbrecht et al., The Plant Cell 1:671680, 1989; Bevan et al., Nucleic Acids Res. 11:369 385, 1983) and the like. The vector and/or other construct can also include regulatory elements. Examples of such regulatory elements include the Adh intron 1 (Callis et al., Genes and Develop. 1:1183 1200, 1987), the sucrose synthase intron (Vasil et al., Plant Physiol. 91:1575 1579, 1989), and the TMV omega element (Gallie et al., The Plant Cell 1:301311, 1989). The vector and/or other construct can also include a selectable marker, a screenable marker and/or other elements as appropriate. Examples of these elements and markers mentioned herein are known in the art and can be readily used without undue experimentation in the methods and constructs described herein.

As used herein “conservative amino acid substitutions” are those that result in variants and equivalents that retain their functionality, for example, the substitution of one or more amino acids by similar amino acids, e.g., the substitution of an amino acid within the same general class, such as an acidic amino acid, a basic amino acid, or a neutral amino acid, by another amino acid within the same class.

As used herein, “probe” means an oligonucleotide or short fragment of DNA designed to be sufficiently complementary to a sequence in a denatured nucleic acid to be probed and to be bound under selected stringency conditions.

As used herein, a “stringent condition” is functionally defined with regard to hybridization of a nucleic-acid probe to a target nucleic acid (i.e., to a particular nucleic acid sequence of interest) by the specific hybridization procedure discussed in Sambrook et al. (Molecular Cloning: A Laboratory Manual, Cold Spring Harbor Laboratory Press, Cold Spring Hobart, 1989, at 9.52 9.55). Regarding the amplification of a target nucleic acid sequence (e.g., by PCR) using a particular amplification primer pair, “stringent conditions” are conditions that permit the primer pair to hybridize substantially only to the target nucleic acid sequence to which a primer having the corresponding wild-type sequence (or its complement) would bind so as to produce a unique amplification product. For hybridization of a probe or primer from one plant species to a polynucleotide of another plant species in order to identify homologs, preferred hybridization and washing conditions are as discussed in Sambrook et al (supra, at 9.47 9.57, wherein “highly stringent conditions” include hybridization at 65° C. in a hybridization solution that includes 6 times SSC and washing for 1 hour at 65° C. in a wash solution that includes 0.5 times SSC, 0.5% SDS. “Moderate stringency” conditions are similar except that the temperature for the hybridization and washing steps is a lower temperature at which the probe is specific for a target sequence, such as at least 42° C., or at least 50° C., or at least 55° C., or at least 60° C. Alternatively, “highly-stringent conditions can include hybridization under conditions comprising a buffer comprising 50% formamide at about 37° C. to 42° C.; or, 42° C. in 50% formamide, 5×SSPE, 0.3% SDS, and a wash step comprising use of a buffer comprising 0.15 NaCl for 15 min at 72° C.

Two nucleic acid sequences are “genetically linked” when the sequences are in linkage disequilibrium.

As used herein, a “tissue sample” is any sample that comprises more than one cell. In a preferred aspect, a tissue sample comprises cells that share a common characteristic (e.g., derived from a leaf, root, or pollen, or from an abscission layer, etc.).

As used herein, a “3′ untranslated region” or “3′ untranslated nucleic acid sequence” or “3′ transcriptional termination signal” refers to the 3′ end of a piece of transcribed but untranslated nucleic acid sequence that functions in a plant cell to cause transcriptional termination and/or the addition of polyadenylate nucleotides to the 3′ end of the RNA sequence being produced. Typically, a DNA sequence located from four to a few hundred base pairs downstream of the polyadenylation site serves to terminate transcription. The region is required for efficient polyadenylation of transcribed messenger RNA (mRNA). RNA polymerase transcribes a coding DNA sequence through a site where polyadenylation occurs.

As used herein, “transformation” refers to the transfer of a nucleic acid sequence into the genome of a host organism such as a host plant, resulting in genetically stable inheritance. Transformed host plants containing the nucleic acid sequences are referred to as “transgenic plants.”

The term “is associated with” as used herein in the context of the Aphis glycines resistance trait being “associated with” a marker, means that the trait locus has been found, using marker-assisted analysis, to be present in soybean plants showing Aphis glycines resistance in live bioassays as described herein.

The term, “modification of the nucleic acid sequence,” refers to modification of a nucleic acid sequence, such as the Rag1 nucleic acid sequence described herein, by techniques such as site-directed mutagenesis. Such techniques allow one or more of the amino acids encoded by a nucleic acid molecule to be altered (e.g. a Cysteine to be replaced by a Tyrosine). Specific techniques include cassette mutagenesis (Wells et al., Gene 34:315 23, 1985), primer extension (Gilliam et al., Gene 12:129 137, 1980; Zoller and Smith, Methods Enzymol. 100:468 500, 1983; Dalbadie-McFarland et al., Proc. Natl. Acad. Sci. (U.S.A.) 79:6409 6413, 1982) and methods based upon PCR (Scharf et al., Science 233:1076 1078, 1986; Higuchi et al., Nucleic Acids Res. 16:73517367, 1988). Site-directed mutagenesis strategies have been applied to plants in vitro as well as in vivo.

An “allele” is any of one or more alternative forms of a gene, all of which alleles relate to one trait or characteristic. In a diploid cell or organism, the two alleles of a given gene occupy corresponding loci on a pair of homologous chromosomes. The Rag1 and Rag2 genes can be allelic to each other.

“Germplasm” means the genetic material with its specific molecular and chemical makeup that comprises the physical foundation of the hereditary qualities of an organism. As used herein, germplasm includes seeds and living tissue from which new plants can be grown: or, another plant part, such as leaf, stem, pollen, or cells, that can be cultured into a whole plant Germplasm resources provide sources of genetic traits used by plant breeders to improve commercial cultivars.

“Hybrid plant” means a plant offspring produced by crossing two genetically dissimilar parent plants.

“Inbred plant” means a member of an inbred plant strain that has been highly inbred so that all members of the strain are nearly genetically identical.

“Introgression” means the entry or introduction by hybridization of a gene or trait locus from the genome of one plant into the genome of another plant that lacks such gene or trait locus.

“Molecular marker” is a term used to denote a nucleic acid or amino acid sequence that is sufficiently unique to characterize a specific locus on the genome. Examples include restriction fragment length polymorphisms (RFLPs) and single sequence repeats (SSRs). RFLP markers occur because any sequence change in DNA, including a single base change, insertion, deletion or inversion, can result in loss (or gain) of a restriction endonuclease recognition site. The size and number of fragments generated by one such enzyme is therefore altered. A probe that hybridizes specifically to DNA in the region of such an alteration can be used to rapidly and specifically identify a region of DNA that displays allelic variation between two plant varieties. SSR markers occur where a short sequence displays allelic variation in the number of repeats of that sequence. Sequences flanking the repeated sequence can serve as polymerase chain reaction (PCR) primers. Depending on the number of repeats at a given allele of the locus, the length of the DNA segment generated by PCR will be different in different alleles. The differences in PCR-generated fragment size can be detected by gel electrophoresis. Other types of molecular markers are known. All are used to define a specific locus on the soybean genome. Large numbers of these have been mapped. Each marker is therefore an indicator of a specific segment of DNA, having a unique nucleotide sequence. The map positions provide a measure of the relative positions of particular markers with respect to one another. When a trait is stated to be linked to a given marker it will be understood that the actual DNA segment whose sequence affects the trait generally co-segregates with the marker. More precise and definite localization of a trait can be obtained if markers are identified on both sides of the trait. By measuring the appearance of the marker(s) in progeny of crosses, the existence of the trait can be detected by relatively simple molecular tests without actually evaluating the appearance of the trait itself, which can be difficult and time-consuming, requiring growing up of plants to a stage where the trait can be expressed.

“Linkage” is defined by classical genetics to describe the relationship of traits that co-segregate through a number of generations of crosses. Genetic recombination occurs with an assumed random frequency over the entire genome. Genetic maps are constructed by measuring the frequency of recombination between pairs of traits or markers. The closer the traits or markers lie to each other on the chromosome, the lower the frequency of recombination, the greater the degree of linkage. Traits or markers are considered herein to be linked if they generally co-segregate. A 1/100 probability of recombination per generation is defined as a map distance of 1.0 centiMorgan (10 cM). Preferably markers useful for screening for the presence of Aphis glycines resistance map to within about 20 cM of the trait, or within about 10 cM of the trait or within about 5 cM of the trait. A second marker that maps to within about 10 cM of a first marker that co-segregates with the Rag1 trait and generally co-segregates with the Rag1 trait is considered equivalent to the first marker. Any marker that maps within 10 cM or 5 cM of the Rag1 trait belongs to the class of preferred markers for use in screening and selection of soybean germplasm having the Rag1 Aphis glycines resistance trait. A number of markers are known to the art to chromosome 7 on which the Rag1 gene is found. A number of markers are proprietary markers known only to certain of those skilled in the art of soybean plant breeding. A proprietary marker mapping within about 10 cM, or about 5 cM, of any publicly known-marker specified herein is considered equivalent to that publicly-known marker.

“Linkage group” refers to traits or markers that generally co-segregate. A linkage group generally corresponds to a chromosomal region containing genetic material that encodes the traits or markers.

“Rag1 resistance” or “Rag1-derived resistance” means resistance in a soybean germplasm to Aphis glycines that is provided by the heterozygous or homozygous expression of the Rag1 gene by soybean germplasm, as demonstrated by resistance to Aphis glycines after inoculation with same according to the methods described herein.

“Self-crossing or self-pollination” is a process through which a breeder crosses hybrid progeny with itself, for example, a second generation hybrid F2 with itself to yield progeny designated F2:3.

As used herein, “regeneration” refers to the process of growing a plant from a plant cell or tissue (e.g., plant protoplast or explant). The regeneration, development, and cultivation of plants such as soybean plants from transformants or from various transformed explants containing a foreign, exogenous gene that encodes a protein of interest are well known in the art (Weissbach and Weissbach, In: Methods for Plant Molecular Biology, Eds, Academic Press, Inc. San Diego, Calif., 1988). This regeneration and growth process can include the steps of selection of transformed cells containing exogenous Rag1 genes and culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

A “modified Rag1 nucleic acid molecule” is used herein to describe embodiments in which the Rag1 nucleic acid molecules are modified by site-directed mutagenesis strategies or other means known to the art. They can be used to confer or contribute to conferring aphid resistance to plants lacking such resistance, or they can be used as nucleic acid molecules to target other nucleic acid molecules, e.g., for further modification. The Rag1 protein that is encoded by the modified Rag1 nucleic acid is referred to as a “modified Rag1 protein.” It is understood that mutants with more than one altered nucleotide can be constructed using techniques that practitioners skilled in the art are familiar with such as isolating restriction fragments and ligating such fragments into an expression vector (see, for example, Sambrook et al., Molecular Cloning: A Laboratory Manual, Cold Spring Harbor Press, 1989). Modified Rag1 nucleic acids and amino acids that can confer Rag1 resistance on a plant are equivalents of the Rag1 nucleic acids specifically described herein, and are included within the scope of the claims hereof. The coding sequence of the Rag1 gene hereof can be extensively altered, for example, by fusing part of it to the coding sequence of a different gene to produce a novel hybrid gene that encodes a fusion protein or chimeric protein. A chimeric protein can be made by a conventional method available in the art, see, for example, Sambrook et al., Molecular Cloning: A Laboratory Manual, Cold Spring Harbor Press, 1989. A chimeric protein hereof can be made by combining any two available aphid-resistance nucleic acid sequences that encode aphid resistance proteins. In one example of the present invention, the chimeric protein can be produced by fusing all or part of the soybean Rag1 nucleic acid sequence that encodes C-terminal portion of the soybean Rag1 protein to all or parts of nucleic acid sequences that encode aphid resistance in other plants or all or parts of nucleic acid sequences encoding other aphid resistance proteins in soybean (see, e.g., Hill, C. B. et al, “Inheritance of Resistance to the Soybean Aphid in Soybean PI 200538,” Crop Sci. 49:1193-1200 (2009)). All such constructs are included within the scope of the claims hereof.

The following examples further demonstrate several preferred embodiments of the present invention. Those skilled in the art will recognize numerous equivalents to the specific embodiments described herein. Such equivalents are intended to be within the scope of the present invention and claims. Although methods and materials similar or equivalent to those described herein can be used in the practice or testing of the present invention, suitable methods and materials are described below. Standard recombinant DNA and molecular cloning techniques used herein are well known in the art.

### EXAMPLES

Unless otherwise defined, all technical and scientific terms used herein have the same meaning as commonly understood by one of ordinary skill in the art to which this invention belongs.

Using SNP markers detected by microarray hybridization, the map position of Rag1 on Linkage Group M was refined (Kaczorowski, K. A., et al., “Microarray-Based Genetic Mapping Using Soybean Near-Isogenic Lines and Generation of SNP Markers in the Rag1 Aphid-Resistance Interval,” Plant Genome 1:89-98 (2008)).

### Example 1

### Fine Mapping the Soybean Aphid Resistance Gene Rag1 in Soybean

The Rag1 gene from Dowling was previously mapped to a 12 centiMorgan (cM) region on soybean chromosome 7 between the simple sequence repeat (SSR) markers Satt435 and Satt463 by Li Y. et al., “Soybean aphid resistance genes in the soybean cultivars Dowling and Jackson map to linkage group M,” Molecular Breeding 19:25-34 (2007). See also U.S. Patent Publication No. 20060015964. To find additional markers near Rag1 (Kaczorowski, K. A., et al., “Microarray-Based Genetic Mapping Using Soybean Near-Isogenic Lines and Generation of SNP Markers in the Rag1 Aphid-Resistance Interval,” Plant Genome 1:89-98 (2008)), hybridized nuclear DNA of the recurrent parent ‘Dwight’, the donor parent Dowling, and a pair of backcross-derived isolines that differed for the Rag1 region, unto Affymetrix soybean GeneChip microarrays. These hybridizations revealed 15 single-feature polymorphisms (SFPs) closely linked to Rag1, of which 12 were confirmed through sequence analysis. Single nucleotide polymorphism (SNP) genotyping assays were developed and four SNPs were mapped to the Rag1 region.

The objective of this study was to fine map the location of Rag1. This fine mapping is useful for marker-assisted selection (MAS) as markers identified during this process that are closely linked to Rag1 almost perfectly segregate with the gene. In addition, the fine mapping aids in gene cloning efforts by positioning the gene into a small interval containing few candidate genes.

Materials and Methods

Plant Material

The fine mapping was initiated by first identifying recombinants near Rag1 in populations of BC₄F₇ plants that were segregating for the soybean aphid-resistance gene. Populations were developed through four backcrosses using the maturity group (MG) VIII cultivar Dowling (PI 548663) (Craigmiles, J. P. et al., “Registration of Dowling soybean,” Crop. Sci. 18:1094 (1978)) as the donor parent of Rag1 (Hill, C. B. et al., “Resistance to the soybean aphid in soybean germplasm,” Crop Sci. 44:98-106 (2004), Li Y. et al., “Soybean aphid resistance genes in the soybean cultivars Dowling and Jackson map to linkage group M,” Molecular Breeding 19:25-34 (2007) and the MG II cultivar Dwight (PI 587386) (Nickell et al., “Registration of ‘Dwight’ Soybean,” Crop Sci. 38:1398 (1998)) as the aphid-susceptible recurrent parent. Dwight was released because of its resistance to soybean cyst nematode (SCN) (Heterodera glycines Ichinohe) and its high yield compared to other cultivars of similar maturity (Nickell et al., “Registration of ‘Dwight’ Soybean,” Crop Sci. 38:1398 (1998)).

The BC₄F₂ populations were developed by first crossing Dowling and the MG II cultivar Loda (Nickell. C. D. et al. “Registration of ‘Loda’ soybean, Crop Sci. 41:589-590 (2001)). Rag1 was initially mapped in this F population (Li Y. et al. “Soybean aphid resistance genes in the soybean cultivars Dowling and Jackson map to linkage group M,” Molecular Breeding 19:25-34 (2007). An aphid-resistant F₂ plant was selected and four backcrosses to Dwight were then performed. The marker Satt435 was used to select for Rag1 during the backcrossing process. Several BC₄F₁ plants were grown and BC₄F₂ seeds were planted in the field. A total of 824 BC₄F₂ plants were screened with the SSR markers Satt463 and Satt540, which flank Rag1. One hundred and eleven plants with recombination events between the markers were selected and harvested for retesting with additional markers.

After the first set of recombinants was analyzed, a set of 1,000 BC₄F₃ plants that had the same pedigree as the BC₄F₂ plants described above were screened with two SNP markers to identify new recombination events close to Rag1. Plants used in this second screening were derived from five BC₄F₂ plants from the first set that were heterozygous for Satt463, Satt435, and Satt540. BC₄F₃ plants were initially screened with TaqMan markers developed for the SNPs ss107918249 and ss107913360 which flank Rag1.

Selected recombinants were then genotyped with additional markers which mapped closer to Rag1. From these screenings, one plant with a key recombination event was selected and grown to produce seed.

DNA Extraction and Quantification

Genomic DNA from the 824 BC₁F₂ plants and an additional 1000 BC₄F₃ plants was extracted from leaves prior to full expansion by a quick DNA extraction method (Bell-Johnson, B. et al. “Biotechnology approaches to improving resistance to SCN and SDS: methods for high-throughput marker-assisted selection,” Soybean Genet. Newsl. 25:115-117 (1998)). Genomic DNA from the 111 selected BC₄F_{2:3} recombinant lines was extracted from young trifoliate leaf tissue bulked from 12 BC₄F₃ progeny plants using the CTAB method described by Keim and Shoemaker, “A rapid protocol for isolation soybean DNA,” Soybean Genet. Newsl. 15:150-152 (1988) with slight modifications.

After the completion of aphid resistance bioassays for the selected BC₄F_{3:4} recombinant line and 11 selected BC₄F_{2:3} recombinant lines, genomic DNA from each of the 44 plants in the bioassay was extracted by the CTAB method as described above. All CTAB DNA was quantified and diluted as described by Kaczorowski, K. A., et al., “Microarray-Based Genetic Mapping Using Soybean Near-Isogenic Lines and Generation of SNP Markers in the Rag1 Aphid-Resistance Interval,” Plant Genome 1.89-98 (2008).

Genetic Mapping and Marker Development in the Rag 1 Interval

Mapping and development of markers near Rag1 was done in three rounds. The first round of mapping was carried out with the SSR markers Satt463, Satt540, and Satt435, which were previously found closely linked to Rag1 (Li Y. et al., “Soybean aphid resistance genes in the soybean cultivars Dowling and Jackson map to linkage group M,” Molecular Breeding 19:25-34 (2007)). The primer sequences for the SSR markers were available from the Choi, I. Y. et al., “A soybean transcript map: gene distribution, haplotype and single-nucleotide polymorphism analysis,” Genetics 176:685-696 (2007), Soybean Linkage Map available on the USDA Soybean Linkage website (http://bfgl.anri.barc.usda.gov/cgi-bin/soybean/Linkage.pl.

Polymerase chain reaction (PCR) was performed according to Cregan, P. B. and Quigley. C. V., “Simple sequence repeat DNA marker analysis. In: Caetano-Anolles, G., et al. and Gresshoff, P. M. (eds), DNA Markers: Protocols, applications and overview. John Wiley & Sons, New York, pp 173-185 (1997), and gel electrophoresis was conducted as described by Kaczorowski, K. A., et al., “Microarray-Based Genetic Mapping Using Soybean Near-Isogenic Lines and Generation of SNP Markers in the Rag1 Aphid-Resistance Interval,” Plant Genome 1:89-98 (2008). The second round of linkage analysis was carried out with the seven SNP markers: 46169.7, 65906.2, 7623, 86377, 442-1688, ss107918249, and ss107913360. The first four of these markers were developed through hybridization of nuclear DNA onto Affymetrix soybean GeneChip microarrays (Kaczorowski, K. A., et al., “Microarray-Based Genetic Mapping Using Soybean Near-Isogenic Lines and Generation of SNP Markers in the Rag1 Aphid-Resistance Interval,” Plant Genome 1:89-98 (2008)). The fifth marker, 442-1688, was developed by re-sequencing PCR products using primers designed from the sequence of an early draft of the soybean genome sequence. The remaining markers, ss107918249 and ss107913360, were developed by re-sequencing sequence tagged sites (STSs) (Hyten D. L. et al., “A high-density integrated genetic linkage map of soybean and the development of a 1,536 Universal Soy Linkage Panel for QTL mapping,” Crop Sci. (submitted) (2009)).

The physical location of the SSR and SNP markers on chromosome 7 was determined from a BLAST search of the primer and consensus sequences of the markers onto the soybean genome sequence available from the Soybean Genome Project, Department of Energy's Joint Genome Institute (www.Phytozome.net). Early genomic SNP discovery was performed using pre-release versions of the soybean draft genome sequence at 4× and 7× coverage, which was kindly supplied by Jeremy Schmutz, Joint Genome Institute, Stanford University Genome Sequencing Center.

SNPs were genotyped with TaqMan SNP assays and MCA using a SNP-specific melt-curve probe with the LightCycler© 480 System, Roche Diagnostics, Indianapolis, Ind., USA, at the University of Illinois Genetic Marker Center (Tables 2 and 3). TaqMan assays and MCAs were performed as described by Kaczorowski, K. A., et al., “Microarray-Based Genetic Mapping Using Soybean Near-Isogenic Lines and Generation of SNP Markers in the Rag1 Aphid-Resistance Interval,” Plant Genome 1:89-98 (2008).

Re-Sequencing of the Rag1 Interval Based on the Draft Soybean Genome Sequence

The third round of linkage analysis was carried out by identifying SNPs in selected intervals between SNP marker ss107918249 and Satt435 based on the draft soybean genome sequence. This region was targeted for re-sequencing based on results from the second round of linkage analysis. SNPs were identified through direct re-sequencing or melt-curve analysis followed by resequencing. Primer pairs were designed using Perl scripts developed to identify primer pairs at 10 kb spacing across large intervals, or the IDT SciTools PrimerQuest^{SM} software tool for single primer pairs, and were ordered from Integrated DNA Technologies (IA, USA). The uniqueness of each primer pair was checked by BLAST search against the soybean draft genome sequence available at the time of design. After identifying of the location of Rag1 by SNP markers 46169.7 and 21A, direct re-sequencing of the region between the markers was conducted to develop additional genetic markers that are more closely flanking the gene. Nine target amplification primer pairs were designed within the region based on the Williams 82 8× assembly (Glyma1).

For direct re-sequencing of the target region, gel electrophoresis of the PCR products was initially run to verify that a single PCR product was produced from each primer pair. If primer pairs produced no product or multiple products, they were reamplified with either a lower (no product) or a higher (multiple products) annealing temperature (Choi, I. Y. et al., “A soybean transcript map: gene distribution, naplotype and single-nucleotide polymorphism analysis,” Genetics 176:685-696 (2007)). After gel electrophoresis on a 0.9% TAE gel, PCR products from the two patents were purified with the QIAquick Gel extraction kit (Qiagen, CA, USA). Purified PCR products were sequenced from both ends using the same primers as used for PCR amplification with the ABI BigDye Terminator v3.1 cycle sequencing kit on an ABI PRISM 3730 sequencer (Applied Biosystems, Foster City, Calif., USA) at the University of Illinois Keck Center Core Facility. To detect SNPs between the two parents, ABI trace files were analyzed by Sequencher version 4.7 (Gene Codes Corporation, Ann Arbor, Mich., USA). Sequences of confirmed SNPs were used to design target amplification primers and probes for TaqMan assays or MCAs.

SNP Discovery with Melt-Curve Analysis

PCR primer pairs generating products less than 200 bp in size and with a 10 kb approximate spacing across the Rag region of the draft soybean genome sequence were designed using a Perl script (available from the authors) and synthesized by Integrated DNA Technologies, IA, USA. PCR and post-amplification melt analysis of samples were performed in 10-μL reaction volumes in 384 well plates on the Roche LightCycler® 480 System (Roche Applied Science), with a pre-incubation at 95° C. 10-minute hold followed by 45 cycles of 95° C. 10 s hold, 54° C. 15 s hold, and 72° C. 20 s hold, with a single fluorescent reading at each extension step. Reactions contained 3 mM MgCl₂ and a final concentration of 1× LightCycler® 480 High Resolution Master Mix (Roche Applied Science). DNA concentration for all reactions was 62.5 ng. Heteroduplex samples were created by spiking with 12.5 ng reference DNA (20% of total DNA). The primers were at 0.25 μM final concentration each. Melting analysis was performed with the Roche LightCycler® 480 immediately following the PCR amplification with an additional denaturation at 95° C. 1 minute hold, cooling at a programmed rate of 2.5° C./s to 40° C. with a 1 minute hold, and a continuous melting curve fluorescent acquisition during a 1° C./s ramp to 95° C. A derivative melting curve plot was obtained with the use of the LightCycler® 480 Gene Scanning software (Roche Applied Science).

### Soybean Aphid Resistance Bioassays

Eleven BC₄F_{2:3} recombinant lines with unique recombination events in the Rag1 interval selected from the 111 BC₄F₂ recombinants in the second round of linkage analysis and one BC₄F_{3:4} line selected in the third round analysis were evaluated for aphid resistance in choice tests. These tests were conducted in an environmental plant growth chamber with temperatures ranging from 22 to 25° C. and 14 h daily illumination at 30 μmol m² s⁻¹ photosynthetically active radiation. Individual plants were grown in 60 by 60 by 60 mm plastic 48-pot inserts (Hummert Intl., Earth City, Mo.) contained inside plastic trays without holes (Hummert Intl., no. F1020) as described by Hill, C. B. et al., “Resistance to the soybean aphid in soybean germplasm,” Crop Sci. 44:98-106 (2004). Each 48-pot insert included 44 plants from the selected recombinant BC₄F_{2:3} lines or BC₄F_{3:4} line and two replicates of the parents, Dowling and Dwight. The 48 plants in an insert were arranged in a complete randomized design (CRD). Aphid inoculation was conducted by placing leaves of Williams 82 that were each infested with 50 to 200 aphids on top of V₁-stage soybean seedlings (Fehr, W. R. et al., “Stage of development descriptions for soybeans, Glycine max (L.) Merrill,” Crop Sci. 11:929-931 (1971)). The aphids included all summer aphid stages of soybean aphid biotype 1 which was collected in Illinois (Kim, K. S. et al., “Discovery of soybean aphid biotypes,” Crop Sci. 48:923-928 (2008); Hill, C. B. et al., “Inheritance of resistance to the soybean aphid in soybean PI 200538,” Crop Sci. 49:1193-1200 (2009)). Soybean aphid colonization was evaluated at 10 and 15 d after aphid infestation by counting the total number of aphids on each plant (Kim, K. S. et al., “Discovery of soybean aphid biotypes,” Crop Sci. 48:923-928 (2008)).

Genetic Mapping

Linkage analysis was conducted with JoinMap 3.0 software (Van Ooijen, J. W. and Voorrips, R. E., “JoinMap 3.0 software for the calculation of genetic linkage maps,” Plant Research International, Wageningen (2001)) using the Kosambi mapping function. A logarithm (base 10) of the odds (LOD) score of 5.0 was used as the threshold to group markers into linkage groups. All 824 BC₄F₂ plants were tested with the SSR markers Satt463 and Satt540 and a Chi-square (χ2) test was used to evaluate segregation of both markers. The 111 BC₄F₂ plants with recombination events between the two SSR markers were screened with the SSR marker Satt435. Plants with recombination events detected between Satt463 and Satt435 or between Satt435 and Satt540 were then tested with all of the SNP markers in the recombinant intervals. For the plants without recombination events in an interval, the genotypes for the markers flanking the interval were used to predict SNP or SSR marker genotypes within the interval.

Each plant in the BC₄F_{2:3} or BC₄F_{3:4} lines that was evaluated for aphid resistance test was also screened with a segregating marker from the Rag1 interval. Genetic associations between the markers and aphid resistance were analyzed by single-factor analysis of variance with the PROC GLM procedure of SAS (SAS Institute, “The SAS System for Windows,” Release 9.00,” (2002)).

Results

Analysis of Recombination Events in the Rag1 Region

The Rag1 locus was previously mapped between the SSR markers Satt540 and Satt463 on soybean chromosome 7 [formerly linkage group (LG) M] (Li Y. et al., “Soybean aphid resistance genes in the soybean cultivars Dowling and Jackson map to linkage group M,” Molecular Breeding 19:25-34 (2007)). To more finely map the location of Rag1, we first tested 824 BC₄F₂ plants with these two markers to identify genetic recombinants near the gene in the first round of linkage analysis. The BC₄F₂ plants were developed by backcrossing Rag1 into Dwight and the BC₄F₁ plant used to develop the population was heterozygous for Rag1, resulting in segregation of the gene in the population. Results from a χ2 test for these markers in the BC₄F₂ population show that Satt540 fit a 1:2:1 segregation ratio for a co-dominant marker but Satt463 did not (P<0.05). The lack of fit for Satt463 was the result of identifying fewer heterozygous plants for this marker than expected, with there being 230 homozygous resistant, 366 heterozygotes, and 222 homozygous susceptible plants in the population. Satt463 did fit a 3:1 segregation ratio when the homozygous resistant and heterozygotes were combined into a single class.

One hundred and eleven BC₄F₂ plants with recombinations between Satt463 and Satt540 were selected and screened with markers from recombinant intervals in the second round of linkage analysis. The markers tested included the SNP markers 46169.7, 65906.2, 7623, 86377, 442-1688, ss107918249, and ss107913360, and the SSR marker Satt435. The distance between Satt463 and Satt540 in the population was 7.41 cM and the largest marker interval was 4.71 cM between Satt463 and ss107918249 (FIG. 2). The relative order of the markers and genetic distances between them was consistent with their physical position on the Williams 82 chromosome 7 draft genome sequence found at the world-wide web address: (phytozome.net/soybean).

Eleven of the 111 BC₄F_{2:3} recombinant lines were selected for aphid resistance testing based on the presence of unique recombination events in the Rag region. Results from evaluating these selected lines for resistance and with the three SSR and seven SNP markers indicates that Rag1 is in a 435 kb interval between ss107918249 and Satt435 (Table 1).

The left border of the position of the gene was identified through the analysis of lines 12 and 72. Both lines segregate for the region right of the recombination point and there was a significant (P<0.0001) association between aphid resistance and segregation of the SNP marker 46169.7, indicating that Rag1 must be to the right of ss107918249. The right border of the position of Rag1 was demonstrated to be between SNP marker 65906.2 and Satt435. This border is shown by lines 6 and 100. Both lines are segregating for 65906.2 and genetic regions to the left of this marker and there was a significant association between aphid resistance and 65906.2 in both lines, showing that Rag1 was segregating in both and therefore left of Satt435.

Saturation of the Rag1 Region with Additional SNP Markers

The region of the soybean draft genome sequence defined by the markers described above as containing the Rag1 locus did not contain a sufficient number of known SNPs to narrow the locus to the smallest interval possible with the large mapping population available. Thus, in the third round of mapping additional SNP markers were developed that could be used to better define the Rag1 region using one of two methods: methods: dye terminator resequencing or melt curve SNP discovery. A total of 79 primer pairs were developed that produced products of approximately 1 kb in size, and these were directly sequenced using capillary dye-terminator chemistry. While PCR and sequencing from intergenic regions of the soybean genome was frequently not successful, this method produced 11 new SNP markers. To supplement the SNPs detected by direct sequencing, 20 primer pairs were designed which produced products less than or equal to 200 bp pairs in size. These primers were spaced at 10 kb intervals throughout a 200 kb region, and produced much more robust PCR products from intergenic DNA than the 1 kb products used for the direct sequencing approach. Using the Roche LightCycler® melt curve SNP detection system, these products were screened for polymorphisms indicated by the formation of heteroduplexes in the presence of Dwight DNA. The LightCycler® Gene Scanning software predicted five of the twenty products contained polymorphisms, and four products were confirmed to contain polymorphisms by direct sequencing. The 15 products which were not predicted to contain polymorphisms were also sequenced, and of these one was found to contain a SNP polymorphism not detected by the melt curve screen and the other 14 were identical in Dowling and Dwight. Thus, the empirically determined false discovery rate was 20% and the false negative rate was 7%.

The melt curve method added a further five polymorphisms (four SNPs and one indel) to the eleven SNPs discovered by direct sequencing. Of these sixteen polymorphisms within the interval, five primer pairs were chosen based on their location for development of genetic markers. Three of these pairs (56B, 21A, and 83A, see FIG. 2, Table 1, and Table 3) contained SNPs that were appropriate for the development of MCA or TaqMan assays. Neither assay could be developed for the two additional primer pairs (27A and 25A, see FIG. 2 and Table 1) because of deletions near these SNPs. Therefore, the lines were genotyped for these two markers by sequencing bulked DNA from the 44 plants in the resistance assays. The six lines with recombination events between ss107918249 and Satt435 were screened with these SNP markers resulting in a more detailed positioning of the recombination events (Table 1). Data from these new markers adjusted the left-most position of Rag1 to 27A based on line 4 and the right-most position to 21A based on lines 82 and 100. This analysis places Rag1 into a 152 kb region.

TABLE 1
Marker genotypes and aphid resistance reactions of 11 BC₄F_{2.3} lines and one
BC₄F_{3:4} line selected based on locations of recombination events in each line

| | Physical position (Mb)^{†} | | | | | | | | | | | | |
|---|---|---|---|---|---|---|---|---|---|---|---|---|---|
| | 8.244 | 5.899 | 5.645 | 5.608 | 5.578 | 5.546 | 5.509 | 5.493 | 5.479 | 5.468 | 5.464 | 5.421 | 5.227 |
| | First Round Markers | | | | | | | | | | | | |
| | Satt | | | | | | | | | | Satt | | |
| | 463 | | | | | | | | | | 435 | | |
| | Second Round Markers | | | | | | | | | | | | |
| | | | | SNP | SNP | | | | | | | SNP | ss |
| | | ss107918249 | | 46169.7 | 65906.2 | | | | | | | 7623 | 107913360 |
| | Third Round Markers | | | | | | | | | | | | |
| Line | | | 27A | | | 56B | KIM3 | 21A | 25A | 83A | | | |
| 12 | S^{††} | S | H | H | H | H | NT | H | H | H | H | H | H |
| 72 | S | S | H | H | H | H | NT | H | H | H | H | H | H |
| 4 | H | H | H | R | R | R | NT | R | R | R | R | R | R |
| 82 | R | R | R | R | R | R | H | H | H | H | H | H | H |
| 100 | H | H | H | H | H | H | H | R | R | R | R | R | R |
| 6 | H | H | H | H | H | H | NT | H | H | H | R | R | R |
| 48 | S | S | NT | S | S | NT | NT | NT | NT | NT | S | H | H |
| 7 | R | R | NT | R | R | NT | NT | NT | NT | NT | R | R | H |
| 62 | R | R | NT | R | R | NT | NT | NT | NT | NT | R | R | H |
| 73 | R | R | NT | R | R | NT | NT | NT | NT | NT | R | R | R |
| 02 | R | R | NT | R | R | NT | NT | NT | NT | NT | R | R | R |
| K39^{‡‡} | H | H | H | H | | S | NT | S | S | S | S | S | S |

| | Physical position (Mb)^{†} | | | | | | | | | |
|---|---|---|---|---|---|---|---|---|---|---|
| | 5.141 | 5.055 | 4.963 | | | | | | | |
| | First Round Markers | | | | | | | | | |
| | | | Satt | | | | | | | |
| | | | 540 | | | | | | | |
| | Second Round Markers | | | | | | | | | |
| | SNP | | | Aphid number^{‡} | | | | | | |
| | 442- | SNP | | Marker used | Pheno- | | | | | |
| Line | 1688 | 86377 | | In F test | type^{§} | R | H | S | P > F^{¶} | R^{2#} |
| 12 | H | H | H | SNP 46169.7 | Segregat- | 90 | 117 | 629 | <0.0001 | 0.94 |
| | | | | | ing | | | | | |
| 72 | H | H | R | SNP 46169.7 | Segregat- | 80 | 68 | 510 | <0.0001 | 0.96 |
| | | | | | ing | | | | | |
| 4 | R | R | R | ss107918249 | Resistant | 62 | 47 | 57 | 0.2043 | 0.08 |
| 82 | H | H | H | KIM3 | Resistant | 49 | 58 | 55 | 0.2071 | 0.08 |
| 100 | R | R | R | KIM3 | Segregat- | 71 | 62 | 657 | <0.0001 | 0.97 |
| | | | | | ing | | | | | |
| 6 | R | R | R | 83A | Segregat- | 47 | 60 | 628 | <0.0001 | 0.91 |
| | | | | | ing | | | | | |
| 48 | H | H | H | SNP 7623 | Suscep- | 941 | 953 | 984 | 0.2080 | 0.17 |
| | | | | | tible | | | | | |
| 7 | H | H | H | ss107913360 | Resistant | 233 | 308 | 291 | 0.3805 | 0.11 |
| 62 | H | H | H | ss107913360 | Resistant | 233 | 308 | 291 | 0.3805 | 0.11 |
| 73 | H | H | H | SNP442-1688 | Resistant | 240 | 280 | 212 | 0.2155 | 0.13 |
| 02 | R | H | H | SNP 86377 | Resistant | 225 | 184 | 193 | 0.4319 | 0.09 |
| K39^{‡‡} | S | S | S | SNP 46169.7 | Suscep- | 774 | 761 | 770 | 0.9010 | 0.01 |
| | | | | | tible | | | | | |

^{†}Physical position of the markers based on the Williams 82 chromosome 7 genome sequence of the 8x draft assembly (Glymal) available at http://www.phytozome.net. the Mb positions of the SNP markers correspond to the SNP locations and the positions of the SSR markers and dominant marker are the locations of the end sequence of the forward primers
^{‡}Mean number of aphids on each plant predicted to be homozygous resistant (R), heterozygous (H), and homozygous susceptible (S) for Rag1 based on the segregation of the marker listed two columns to the left
§Phenotype of the line based on aphid numbers and the marker association test
¶Significance level of the marker association
^{#}R2 value of the marker assocition.
††Marker genotypes of the BC₄F₂ or BC₄F₃ plants that formed the recombinant lines; R designates that the plant was homozygous for the allele from Dowling, H designates heterozygous, S designates homozygous for the allele from Dwight and NT designates not tested. Highlighting is placed at the genetic interval containing inferred recombination event.
^{‡‡}The line K39 is a BC₄F_{1.4} recombinant line selected from the 1.000 BC₄F₁ heterozygous plants. All other lines in the table are BC₄F₂ #Z,899; lines

TABLE 2
Sequences of target amplification primers and TaqMan probes for
SNP genotyping assays

| SNP NAME | TYPE | PRIMER SEQUENCE | SEQ ID NO. |
|---|---|---|---|
| ss107913360 | Forward | 5′-TCTGTGGTGGCACATCGATT | SEQ ID NO: 18 |
| | Reverse | 5′-TGCCGGTGCTACCATTCTG | SEQ ID NO: 19 |
| | TaqMan Probe 1 | 5′-AAACCACCGAGCCAG-FAM (Dwight) | SEQ ID NO: 20 |
| | TaqMan Probe 2 | 5′-AAACCACGGCGCCAG-VIC (Dowling) | SEQ ID NO: 21 |
| SNP86377 | Forward | 5′-CAGATGAAGACCCAATGATATGTGAGAT | SEQ ID NO: 22 |
| | Reverse | 5′-GGGTGCCACTGTCTTGTTTAAGT | SEQ ID NO: 23 |
| | TaqMan Probe 1 | 5′-CACATGCAGCCAAGCA-VIC (Dwight) | SEQ ID NO: 24 |
| | TaqMan Probe 2 | 5′-CACATGCACCCAAGCA-FAM (Dowling) | SEQ ID NO: 25 |
| ss107918249 | Forward | 5′-TGGTGTTTATTTTCGACCAAAATTGAAGTT | SEQ ID NO: 26 |
| | Reverse | 5′-CTTACATACAAATCTTTAGGCTCCTTATAACCT | SEQ ID NO: 27 |
| | TaqMan Probe 1 | 5′-ATAATCTACATGTAAACATCTAT-VIC (Dowling) | SEQ ID NO: 28 |
| | TaqMan Probe 2 | 5′-ATAATCTACATGTAAACTTCTAT-FAM (Dwightj) | SEQ ID NO: 29 |

TABLE 3
Sequences of target amplification primers and melting curve assay (MCA)
sensor probe for genotyping SNP

| SNP NAME | TYPE | PRIMER SEQUENCE | SEQ ID NO. |
|---|---|---|---|
| SNP7623 | Forward | 5′-GGACTTGGAGAAGAAATTAGCCA | SEQ ID NO: 30 |
| | Reverse | 5′-GCAACATCAAAGGCTCTCACA | SEQ ID NO: 31 |
| | MCA Probe | 5′-ACAGTATGACCAGCAGCTTCCAA-PH | SEQ ID NO: 32 |
| SNP46169.7 | Forward | 5′-AGGAGATGTCATCAATAAAGCC | SEQ ID NO: 33 |
| | Reverse | 5′-TGCTGCCTTGTCTAGACCTAA | SEQ ID NO: 34 |
| | MCA Probe | 5′-ATCAGCAAAACAGATGCAGACGTT-PH | SEQ ID NO: 35 |
| SNP442-1688 | Forward | 5′-CGATGAAATATATCCACTCTTATTAGCA | SEQ ID NO: 36 |
| | Reverse | 5′-ACTAAGGCACATATTCTATATAAAAAAACT | SEQ ID NO: 37 |
| | MCA Probe | 5′-TTGTGTATTACTAATTATATCATCCGTGAAAAGCT-PH | SEQ ID NO: 38 |
| SNP65906.2 | Forward | 5′AGATAACACATTTCAGCGGCTTTCG | SEQ ID NO: 39 |
| | Reverse | 5′-TGATGATGGAGTTGGTGTTGCAGG | SEQ ID NO: 40 |
| | MCA Probe | 5′-CTTCACATTGGCCACCACAACCACA-PH | SEQ ID NO: 41 |
| 56B | Forward | 5′-GCAAGCTAAACATGATTGAAGGAT | SEQ ID NO: 42 |
| | Reverse | 5′-GTTTTGCCTGATTTATTCACTGTTTCAA | SEQ ID NO: 43 |
| | MCA Probe | 5′-GTTGGTTTTCTACGGAATGGTAGTACGCCATCCAT-PH | SEQ ID NO: 44 |
| 21A | Forward | 5′-TCTTGGCTTGTCTTCTATCTTCCAAACGA | SEQ ID NO: 45 |
| | Reverse | 5′-AGATTAAACTTTTGGGCTATGAAACCCAGA | SEQ ID NO: 46 |
| | MCA Probe | 5′-AATTTCCCAAATCCATATGTATTGTACCGATATCA-PH | SEQ ID NO: 47 |
| 83A | Forward | 5′-CATTCGTACCTTCACCGCATTACT | SEQ ID NO: 48 |
| | Reverse | 5′-AAGACACTATGAATCCCTAATCTCATGCCA | SEQ ID NO: 49 |
| | MCA Probe | 5′-AAATAGATAAAAGATTAAAATAAATTTTTTAAAAG-PH | SEQ ID NO: 50 |
| KIM3 | Forward | 5′-AAAGGAAACTAATTCATGTTTGCTCACAAT | SEQ ID NO: 51 |
| | Reverse | 5′-TTTGTGCCCATTTGTTACAGTCTTTCCATA | SEQ ID NO: 52 |
| | MCA Probe | 5′-TATTCTTTTGAAAAGCTGAAACAAACACATGAAAA-PH | SEQ ID NO: 53 |
| KIM5 | Forward | 5′-AACCTGAATTGCCAGCATAAGGGC | SEQ ID NO: 54 |
| | Reverse | 5-AAGCGAGGACCACTTCTGTGTTCT | SEQ ID NO: 55 |

To further narrow the genomic region containing the Rag1 gene, a new set of 1,000 BC₄F₃ plants segregating for the gene were screened with SNP markers to identify additional recombination events, especially recombination events in the interval between SNP markers 27A and 21A. From the 1,000 plants screened, only one new recombination event in this interval was found. This plant had a recombination between 46169.7 and 65906.2 and genetic analysis of progeny from this plant positioned Rag1 right of 46169.7. To further refine the position of Rag1 within the 115 kb region defined by 46169.7 and 21A, re-sequencing of the region was conducted to develop additional genetic markers within the region. Nine target amplification primer pairs were designed and an additional informative SNP marker (KIM3) was developed which further defined the interval by excluding a small additional region. The minimal genetic region containing Rag1 is thus defined by 46169.7 and KIM3. KIM5 is an additional marker developed that maps to the same location as 46169.7.

As before, the physical positions of all of the markers used on the Williams 82 draft genome sequence are consistent with their genetic position in the map of the Rag1 locus, confirming that the assembly of the genome sequence in this region is correct as far as can be assessed using his method, and that the Dowling and Williams 82 genomes do not differ by any large-scale deletion, rearrangement or insertion events within this region. The genomic region of the aphid-susceptible genotype Williams 82 cognate to the region of the Dowling genome containing the Rag1 locus, as defined by genetic markers 46169.7 or KIM5 and KIM3, is 99,551 kb in length (Table 1; FIG. 2).

Discussion

A fine map of the region containing the Rag1 locus was developed. This mapping effort was greatly accelerated by the availability of the soybean genome sequence from the DoE Joint Genome Institute (http://www.phytozome.net/soybean). This sequence information was especially valuable for developing SNP markers through re-sequencing of targeted regions and identifying what genes underlay the interval where the gene is located.

Current gene annotation of the 99.5 kb region containing the Rag1 locus on the Williams 82 genome sequence (http://www.phytozome.net/soybean) identifies the presence of several candidate soybean aphid resistance genes in this region, some of which have their expression supported by EST data. The genes include a small cluster of nucleotide binding leucine-rich repeat (NBS-LRR) genes that are in Rag1.

Sequences of these genes have homology with Arabidopsis genes encoding disease resistance proteins such as the resistance to P. syringae 2 (RPS2) and TIR-NBS-LRR class (http://www.phytozome.net/soybean). The NBS-LRR genes were good candidates for the Rag1-encoding locus because the root knot nematode (Meloidogyne incognita) resistance gene in tomato, Mi, is an NBS-LRR gene (Milligan, S. B. et al. “The root knot nematode resistance gene Mi from tomato is a member of the leucine zipper, nucleotide binding, leucine-rich-repeat family of plant genes,” Plant Cell 10:1307-1320 (1998)) and this gene also confers resistance to potato aphid (Macrosiphum euphorbiae Thomas) (Rossi, M. et al., “The nematode resistance gene Mi of tomato confers resistance against the potato aphid,” Proc. Nat'l Acad. Sci. USA 95:9750-9754 (1998)).

In addition, aphid resistance genes were mapped in NBS-LRR cluster regions in melon (Cucumis melo) and Medicago truncatula (Brotman, Y. et al., “Resistance genes homologous in melon are linked to genetic loci conferring disease and pest resistance,” Theor. Appl. Genet. 104:1055-106 (2002); Klingler, J. et al. “Aphid resistance in Medicago truncatula involves antixenosis and phloem-specific, inducible antibiosis, and maps to a single locus flanked by NBS-LRR resistance gene analogs,” Plant Physiol. 137:1445-1455 (2005)).

Previous research on the plant response to aphid feeding suggested that jasmonic acid (JA)-, ethylene-, and salicylic acid (SA)-regulated signaling pathways were at least partially activated by aphid feeding (Li Y. et al., “Soybean defense responses to the soybean aphid,” New Phytologist 179(1):185-195 (2008)). The genomic region surrounding Rag1 also contains genes homologous to genes induced by the JA and SA pathways, were candidate genes for the Rag1 locus.

We used TaqMan and MCA to genotype SNPs in this study, and direct resequencing and melt curve analysis to discover SNPs. The melt curve analysis with the LightCycler® Gene Scanning system successfully predicted four of five PCR products that contained SNP polymorphisms, with a fifth detected by direct sequencing.

To obtain reliable and reproducible SNP genotyping results, we found that the MCA required relatively high-quality and quantified DNA extracted using the CTAB extraction method, while the TaqMan assays produced high-quality genotypic data even with quick-extracted DNA (Bell-Johnson, B. et al., “Biotechnology approaches to improving resistance to SCN and SDS: methods for high-throughput marker-assisted selection,” Soybean Genet. Newsl. 25:115-117 (1998)). MCA can also be used with other quick DNA extraction protocols. For screening of the 1000 BC₄F₃ plants for recombinants, we used two SNP markers designed for the TaqMan assay because the quick-extraction method required just 3 min to extract DNA from 96 samples. In addition, the quick-extraction method requires a relatively small amount of tissue, so the tissue sampling can be done much earlier during plant development than with the CTAB method. A minor drawback of the TaqMan assay compared to the MCA is that TaqMan assays take approximately 2 hours while MCA takes 1 hour. However, with a 384 well instrument, we were able to test a few thousand samples daily. On the basis of time and labor, the TaqMan assay with quick-extract DNA was the most efficient for MAS due to its simplicity and rapidity when a large number of samples need to be tested with a small number of markers. For fine-scale mapping when markers are tested on a few key recombinants, MCA was also efficient because the lower cost to set up each marker compared to TaqMan assays.

Our high-resolution genetic map of the Rag1 locus is useful to facilitate MAS for Rag1 in cultivar breeding programs. SNPs are becoming the marker of choice in MAS and the markers we identified within the 99.5 kb region that are closely linked to Rag1 are highly effective tools in MAS since only very rare recombinations are anticipated between these markers and the Rag1 locus during selection. Our efforts to fine map the Rag1 locus also greatly facilitated the molecular identification and functional characterization of the Rag1 locus. The cloning of Rag1 contributes to a better understanding of the mechanism of soybean aphid defense. This information can be used to identify other candidate soybean aphid resistance genes using the completed soybean genome sequence, for comparison to cloned insect resistance genes in other species and to introduce aphid resistance into susceptible genotypes using biotechnology approaches.

The appearance of soybean aphid biotype variation in North America (Kim, K. S. et al., “Discovery of soybean aphid biotypes,” Crop Sci. 48:923-928 (2008)) increases the need to stack aphid resistance genes in cultivars. The breakdown of resistance genes by insect pests has frequently occurred, especially when resistance is conditioned by a single gene and cultivars carrying the gene are widely grown (Burd, J. D. and Porter, D. R., “Biotypic diversity in greenbug (Hemiptera: Aphidae): characterizing new virulence and host associations,” (2006); Haley, S. D. et al., “Occurrence of a new Russian wheat aphid biotype in Colorado,” Crop Sci. 44:1589-1592 (2004)). Stacking resistance genes will slow down or delay resistance gene adaptation. The close linkage between Rag1 and the SNP markers we identified and the availability of efficient SNP marker detection assays makes these markers especially useful in MAS and the stacking of Rag1 in combination with other aphid resistance genes.

The sequence of the Rag1 genomic interval [SEQ ID NO:17] identified in this Example is provided herein. This Example also provides gene models based on open reading frames determined using FGENESH (available commercially from Softberry http://linux1.softberry.com/berry.phtml) and freely available for use at http://mendel.cs.rhul.ac.uk/mendel.php?topic=fgen-file), and protein sequences [SEQ ID NOS.1-16] for exons in this genomic interval.

### Example 2

### Physical Mapping of the Aphid Resistant Gene, Rag1 in Soybean Using the Zip Fosmid Contig Construction Method

A large number of single nucleotide polymorphisms (SNPs) in the Rag1 interval were identified using Affymetrix Soybean GeneChip microarrays and genome resequencing. Genetic recombinants were identified in a large population segregating for the Rag1 locus and these recombinants were phenotyped for aphid resistance and genotyped with SNP and SSR markers.

This mapping narrowed the location of the Rag1 locus to a fully sequenced region in the aphid-susceptible cultivar Williams 82. In order to determine the sequence of the resistant cultivar Dowling at this locus, high-coverage Dowling fosmid libraries were constructed, and a fosmid contig was built in the interval region. To facilitate the creation of a fosmid contig spanning the Rag1 region in the resistant biotype, we have developed a library screening method, PCR (ZIP). Data on the sequence of the resistant biotype in the Rag1 interval is provided. The cloning and genomic sequencing of Rag1 provides the first insight into the genome structure and function of soybean aphid resistance genes, allows the discovery of other candidate resistance genes using the recently completed soybean genome sequence, and greatly facilitates the transgenic manipulation of aphid resistance in soybean.

Materials and Methods

Plant Material and DNA Isolation

A population of 824 BC₄F_{2:3} lines were used for recombinant identification and genetic mapping of the Rag1 gene. The aphid resistance tests were conducted in the greenhouse using a non-choice inoculation method. The details of the plant material development and phenotyping were described previously by Kaczorowski. K. A., et al., “Microarray-Based Genetic Mapping Using Soybean Near-Isogenic Lines and Generation of SNP Markers in the Rag1 Aphid-Resistance Interval,” Plant Genome 1:89-98 (2008). To prepare DNA extracts for library construction, the soybean plants were grown in the greenhouse until V5 (Fehr et al. (1971), supra, when the tops of the plants were removed to stimulate new branches. The newly emerged leaf and meristematic tissues were collected from the tips of the branches and frozen immediately in liquid nitrogen. DNA samples were extracted from 5 g of the tissue using the method described by Swaminathan et al. (2007), “Global repeat discovery and estimation of genomic copy number in a large, complex genome using a high-throughput 454 sequence survey,” BMC Genomics 8:132. The genomic DNA used for genotyping of the BC₄F₂ plants and additional BC₄F_{2:3} plants was extracted from leaves prior to full expansion by the quick extraction method (Bell-Johnson, B. et al. “Biotechnology approaches to improving resistance to SCN and SDS: methods for high-throughput marker-assisted selection,” Soybean Genet. Newsl. 25:115-117 (1998)). Genomic DNA of the 115 BC₄F_{2:3} lines in the segregating mapping population and 111 BC₄F_{2:3} selected recombinant lines was extracted from 12 plants from each line using bulked young trifoliate tissues by the CTAB method described by Keim and Shoemaker, “A rapid protocol for isolation soybean DNA,” Soybean Genet. Newsl. 15:150-152 (1988), with modifications.

SNP Marker Development and Genotyping

Primer pairs were designed using a Perl script developed in the lab, using the available Williams 82 whole genome sequence as a design template (http://www.phytozome.net/) in the mapped Rag1 gene interval. The target PCR product size was 1 kb. These primers were used to amplify the corresponding DNA fragments from the resistant Dowling and susceptible Dwight genome. The PCR products were purified using the Qiagen PCR purification kit (Qiagen, CA). The sequencing reactions were set up using 200 ng of PCR product and the BigDye Terminator Cycle Sequencing Kit (Applied Biosystems, Foster City, Calif.) and sequenced at the University of Illinois Keck Center Core Facility. SNPs were identified by comparing the sequences and chromatograms of the PCR products derived from Dowling and Dwight.

Fosmid Library Construction

Dowling genomic DNA fosmid libraries were constructed using CopyControl™ Fosmid Library Production Kit (Epicenter Biotechnologies, WI) following the manufacturer's instructions with some modifications. Briefly, 40 μg of DNA was physically sheared by freeze-thaw 30 times until the DNA fragment size was 35-45 kb. The ends of the sheared DNA fragments were repaired using T4 polymerase and precipitated. For ligation, 2 μl of the end-repaired and precipitated DNA was ligated to the pCC1FOS™ Vector using DNA ligase in a total volume of 10 μl and incubated at room temperature for 2 hours followed by an overnight incubation at 4° C. The 10 μl ligation reaction was packaged using 25 μl packaging extract at 30° C. for 2 hours and then an additional 25 μl packaging extract for another 2 hours. The packaging reaction was then diluted to 1 ml final volume with phage dilution buffer and inactivated by adding 25 μl chloroform. After the titer of packaged fosmid clones was determined, the whole packaged phage particles were adsorbed to freshly prepared EPI300-T1R cells (OD600=0.9). The infected cells were spread on top of nylon membranes in YT agar plates containing 12.5 mg/ml chloramphenicol with an anticipated cfu number of 5000 per plate. The plates were incubated overnight at 37° C. to allow colony growth.

PCR-Based Fosmid Library Screening and Contig Construction Using ZIP

Based on the available whole soybean genome sequence of Williams 82, PCR primers were designed from the Rag1 sequence interval using the Primer3 program (Rozen, S, and Skaletsky, H., “Primer3 on the WWW for general users and for biologist programmers,” Methods Mol. Biol. (2000)). The primers that amplified both Williams 82 and Dowling DNA samples with a clear single band were used for fosmid library screening. The primary fosmid library plates constructed above were then duplicated by using the nylon membranes to inoculate replica plates. The newly-inoculated replica plates were incubated at 37° C. for about 6 hours or until the colony size reached about 1 mm. The cells from the replica plates were then collected by adding 10 ml LB media containing 12.5 mg/ml chloramphenicol and scraping the cells a the plate. A simple PCR-based screening procedure, which we call Zoom In PCR or ZIP (FIG. 2) was applied to screen the positive clones. The pooled cells collected from each plate containing five to six thousand colonies were used directly as a template for PCR after an aliquot was saved as a glycerol stock. If a glycerol stock of a pooled culture produced the expected band, the positive stock was titered and spread to 10-20 sub-plates with 1/10 of the colony number per plate of the original plate (500 to 600 colonies). The pooled cells derived from the 10 sub-plates were screened again by PCR. If positives were detected, the positive glycerol stock was spread to 10-20 sub-sub-plates with an average of 50 colonies in each (if no positives were detected, another set of 10 sub-plates was prepared and screened). The set of sub-sub-plates were replicated before PCR screening of the pooled cells. All 50 colonies in the positive duplicated sub-sub plate were then picked individually and inoculated for single-colony-culture PCR to screen the final positive clones.

### Results and Discussion

Fine-Mapping of the Rag1 Gene

To refine the genetic position of Rag1 for more accurate marker-assisted selection and its eventual cloning, we used the physical map information from the soybean genome project combined with high-throughput marker generation. Kaczorowski, K. A., et al., “Microarray-Based Genetic Mapping Using Soybean Near-Isogenic Lines and Generation of SNP Markers in the Rag1 Aphid-Resistance Interval,” Plant Genome 1:89-98 (2008), identified single nucleotide polymorphism (SNP) markers flanking the Rag1 gene interval using a GeneChip microarray approach. Two of the four identified SNP markers were mapped between the two previous flanked markers Satt435 and Satt463. These SNPs spanned a 540 Mb contiguous sequenced region (Kaczorowski, K. A., et al., “Microarray-Based Genetic Mapping Using Soybean Near-Isogenic Lines and Generation of SNP Markers in the Rag1 Aphid-Resistance Interval,” Plant Genome 1:89-98 (2008)), which was helpful to narrow the gene interval. However, for gene cloning, finer genetic mapping was necessary. To develop more SNP markers in the previously defined Rag1 gene interval, 64 primer pairs were designed across the Williams 82 sequence of the interval region for PCR amplification of Dowling and Dwight followed by the sequence comparison. In total, 34 of the 64 primer pairs successfully amplified a product from both the Dowling and Dwight DNA samples, and 23 of these products were used for sequencing. Sequences of good quality were obtained from 10 pairs of PCR products, horn which 62 SNPs were detected and 16 SNP markers were validated. A fine-mapping effort using these SNP markers (Example 1) narrowed the location of the Rag1 locus to a region sufficiently small to begin fosmid contig construction from the Dowling genotype.

Sequence Annotation of the Fine-Mapped Rag1 Gene Interval from the Susceptible Williams 82 Genotype

The available genome sequence of Williams 82 in the interval region has an average G+C content of 29.7%. The sequence, for which no public annotation was available at the time, was annotated using in-house scripts, BLAST and FGENESH, and contains several genes and repetitive sequences. The repeat sequences accounted for 28.4% of the interval sequence when masked against the repeat database combined of the repeats identified from 3× resequenced soybean genome plus a database of known soybean repeats (Swaminathan et al. (2007), “Global repeat discovery and estimation of genomic copy number in a large, complex genome using a high-throughput 454 sequence survey’, in BMC Genomics 8:13). In total, 14 gene models were predicted in the Rag1 interval. Based on the putative functions of the genes in the interval, it was not immediately apparent that any particular gene was most likely to be the Rag1 gene. To obtain a clone containing the true Rag1 sequence conferring aphid resistance, it was necessary to screen a genomic library and build a physical contig across the region from the resistant genotype, Dowling.

### Fosmid Library Construction

Large insert genomic libraries have been very important for comparative mapping, genome sequencing, and gene cloning. The fosmid family of vectors allows large insert genomic library construction with an average insert size of 40 kb, and these vectors are comparatively quick and cost-effective to construct, sequence, and manipulate. In this study we constructed a fosmid library from the Dowling genotype containing approximately 120,000 unique clones with an average insert size of 39.6 kb. This library represents 4.3× coverage of the soybean genome, given the estimated soybean genome size of 1115 Mb (Arumuganathan, K. and Earle, E., “Nuclear DNA content of some important plant species,” Plant Molecular Biology Reporter 9:208-213 (1991)). Clones in the library were evenly spread onto 22 plates with about 5500 clones per plate for screening. Fosmid library screening using Zoom In PCR (ZIP)

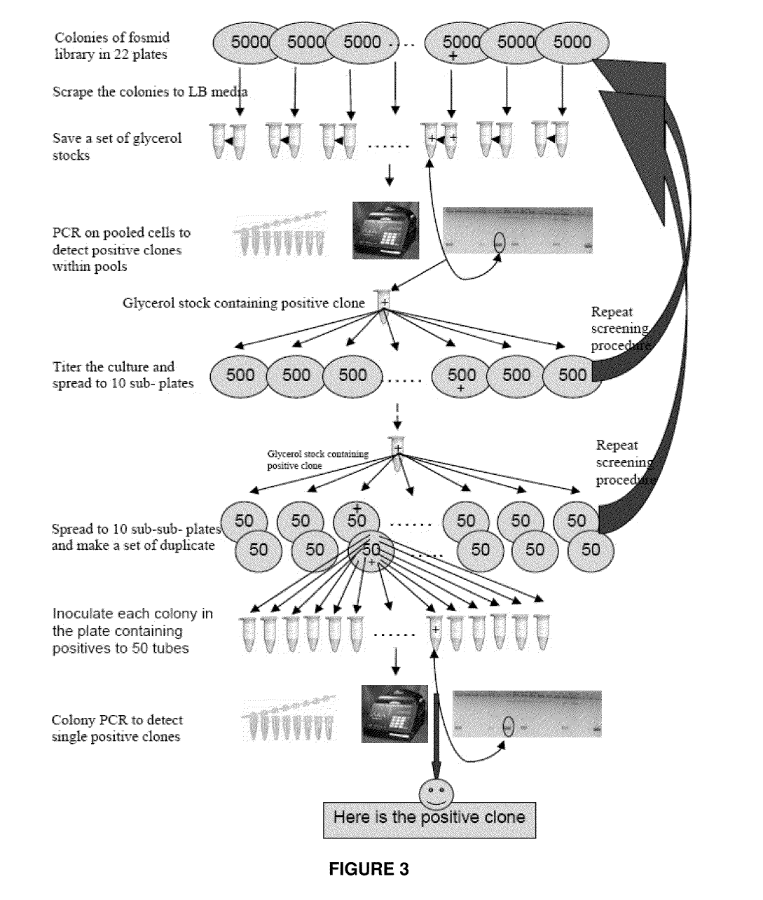

A simple PCR-based library screening method was developed and applied in order to utilize the Williams 82 genome sequence information to produce a fosmid contig spanning the Rag 1 locus from the resistant genotype, Dowling (FIG. 3). Primer pairs were designed across the interval region using the Williams 82 sequence with a distance between primer pairs which ranged from 12 to 30 kb. Primer pairs were validated for use in library screening by generating a single clear PCR band from both Williams 82 and Dowling DNA templates. Each primer pair was used to screen 22 glycerol stocks representing 22 agar plates each with around 5,500 colonies, and glycerols from which positive bands were amplified used to inoculate rounds of sub-plates which were used to narrow down the number of colonies until it was known to be one of 50 colonies, when all remaining colonies were picked and screened individually. Occasionally, homologous regions of the soybean genome generated false positives, but unlike with colony lift hybridization screening, these are readily detected by direct sequencing of the bands produced in the first round of screening.

As an example, the cultures from 7 of the 22 plates produced positive PCR bands with one primer pair. However, there were clearly two different classes of band in these 7 cultures with slightly different sizes. Six of the PCR products were sequenced and compared (FIG. 3). Two of the PCR products showed exactly the same sequences as each other (type 1) while the other four had different sequences from the first two, but the same sequences as each other (type 2). Besides insertions at three places in type 2 sequences compared to the sequence of type 1 (FIG. 4), the similarity of type 1 and type 2 sequences was about 82%. The two types of sequence were blasted to the Williams 82 whole genome sequence (http://www.phytozome.net/) separately. The type 1 sequence aligned perfectly with the Williams 82 genome at the expected position in the Rag1 interval at Gm 07, while the type 2 sequence aligned 100% with the Williams 82 genome within the homologous chromosome Gm 16. The soybean genome has undergone two to three rounds of duplication and the most recent one can occurred 1 to 3 million years ago (Jackson, S. A. et al., “Toward a reference sequence of the soybean genome: a multiagency effort,” Crop. Sci. 46:s55-s61 (2006)). Therefore, some duplicated regions are highly similar and bring manifest obstacles to the construction of contigs using library screening. In this study, the ZIP method combined with the Williams 32 genome sequence enabled us to rapidly exclude the false positives and allowed the rapid detection of multiple contiguous fosmid dones scanning the interval in the resistant biotype. This PCR-based library screening is highly efficient, economical, and applicable for screening of a small number of clones from fosmid libraries, which can be generated rapidly and at low cost. The traditional colony picking and storage, membrane gridding/printing, hybridization, or plasmid DNA isolation and complex DNA pooling procedures were totally eliminated by this simple PCR-based library screening method.

### Claim Interpretation

When a compound is claimed as a composition of matter, it should be understood that compounds known in the art including the compounds or sequences disclosed in the references disclosed herein are not intended to be claimed by themselves. When a Markush group or other grouping is used herein, all individual members of the group and all combinations and subcombinations possible of the group are intended to be individually included in the disclosure.

One of ordinary skill in the art will appreciate that methods, genetic or other elements, starting materials, molecular biological and agronomic methods, other than those specifically exemplified herein can be employed in the practice of the invention without resort to undue experimentation. All art-known functional equivalents, of any such methods, genet c or other elements, starting materials, molecular and agronomic methods, are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing.” or “characterized by,” and is inclusive and open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising,” particularly in a description of components of a composition or in a description of elements of a device, is understood to encompass those compositions and methods consisting essentially of and consisting of the recited components or elements. The invention illustratively described herein suitably can be practiced in the absence of any element or elements, limitation or limitations which are not specifically disclosed herein.

In general the terms and phrases used herein are defined above and/or have their art-recognized meaning, which can be found by reference to standard texts, journal references and contexts known to those skilled in the art.

In addition to the above-discussed procedures, practitioners are familiar with the standard resource materials that describe specific conditions and procedures for the construction, manipulation and isolation of macromolecules (e.g., DNA molecules, plasmids, etc.), generation of recombinant organisms and the screening and isolating of clones (see, for example, Sambrook et al., Molecular Cloning: A Laboratory Manual, Cold Spring Harbor Press, 1989; Mailga et al., Methods in Plant Molecular Biology, Cold Spring. Harbor Press, 1995; Birren et al., Genome Analysis: Analyzing DNA, 1, Cold Spring Harbor, N.Y., 1997).

It will be understood by those skilled in the art that, without departing from the scope and spirit of the above description and without undue experimentation, the invention can be performed within a wide range of equivalent parameters. While the present invention has been described in connection with specific embodiments thereof, it will be understood that it is capable of further modifications. The claims below cover any uses, variations, or adaptations of methods and constructs described herein following the principles disclosed herein in general. Various permutations and combination of the elements provided in all the claims that follow are possible and fall within the scope of the claims.

All publications and patents mentioned in this specification are herein incorporated by reference as if each individual publication or patent was specially and individually stated to be incorporated by reference, to the extent not inconsistent with the disclosure and definitions set forth herein, for purposes of enablement and written description.

### REFERENCES

PCT patent application WO 84/02913
U.S. Patent Publication No. 20060015964, published
U.S. Patent Publication No. US20100083396A1, published 2010-04-01, Hill et al. “Soybean Gene for Resistance to Aphis glycines” U.S. Patent Publication No. US20060015964A1, published 2006-01-19, Hill et al. “Soybean genes for resistance to aphis glycines”
Appleby, Nikki et al. (2009), “New Technologies for Ultra-High Throughput Genotyping in Plants,” Methods in Molecular Biology 513:19-39.
Arumuganathan, K. and Earle, E., “Nuclear DNA content of some important plant species,” Plant Molecular Biology Reporter 9:208-218 (1991)
Bell-Johnson, B. et al., “Biotechnology approaches to improving resistance to SCN and SDS: methods for high-throughput marker-assisted selection,” Soybean Genet. Newsl. 25:115-117 (1998)
Brotman, Y. et al., “Resistance genes homologous in melon are linked to genetic loci conferring disease and pest resistance,” Theor. Appl. Genet. 104:1055-106 (2002)
Burd, J. D. and Porter, D. R., “Biotypic diversity in greenbug (Hemiptera: Aphidae): characterizing new virulence and host associations,” (2006
Chandler et al., The Plant Cell 1:1175 1183, 1989)
Chen, C Y et al., “SSR marker diversity of soybean aphid resistance sources in North America,” Genome 50:1104-1111 (2007)
Choi, I. Y. et al., “A soybean transcript map: gene distribution, haplotype and single-nucleotide polymorphism analysis,” Genetics 176:685-696 (2007)
Craigmiles, J. P. et al., “Registration of Dowling soybean,” Crop. Sci. 18:1094 (1978)
Cregan, P. B. and Quigley, C. V., “Simple sequence repeat DNA marker analysis. In: Caetano-Anolles, G., et al. and Gresshoff, P. M. (eds), DNA Markers: Protocols, applications and overview. John Wiley & Sons. New York, pp 173-185 (1997)
Ebert et al., Proc. Natl. Acad. Sci. (U.S.A.) 84:5745 5749, 1987
Fehr, W. R. et al., “Stage of development descriptions for soybeans, Glycine max (L.) Merrill,” Crop Sci. 11:929-931 (1971)
Haley, S. D. et al., “Occurrence of a new Russian wheat aphid biotype in Colorado,” Crop Sci. 44:1589-1592 (2004)
Hill, C. B. et al, “Inheritance of Resistance to the Soybean Aphid in Soybean PI 200538.” Crop Sci. 49:1193-1200 (2009)
Hill, C. B. et al., “A Single Dominant Gene for Resistance to the Soybean Aphid in the Soybean Cultivar Dowling,” Crop. Sci. 46:1601-1605 (2006)
Hill, C. B. et al., “Resistance to the soybean aphid in soybean germplasm,” Crop Sci. 44:98-106 (2004)
Hill, J. H. et al., “First report of transmission of Soybean mosaic virus and Alfalfa mosaic virus by Aphis glycines in the New World,” Plant Disease 85:561 (2001)
Hyten D. L. et al. (2010), “A High Density Integrated Genetic Linkage Map of Soybean and the Development of a 1536 Universal Soy Linkage Panel for Quantitative Trait Locus Mapping,” Crop Sci. 50:960-968
Jackson, S. A. et al., “Toward a reference sequence of the soybean genome: a multiagency effort,” Crop. Sci. 46:s55-s61 (2006)
Kaczorowski, K. A., e: al., “Microarray-Based Genetic Mapping Using Soybean Near-Isogenic Lines and Generation of SNP Markers in the Rag1 Aphid-Resistance Interval,” Plant Genome 1:89-98 (2008)
Keim and Shoemaker, “A rapid protocol for isolation soybean DNA,” Soybean Genet. Newsl. 15:150-152 (1988)
Kim, K. S. et al., “Discovery of soybean aphid biotypes,” Crop Sci. 48:923-928 (2008)
Kim, K. S. et al., “Fine mapping the soybean aphid resistance gene Rag1 in soybean,” Theor Appl Genet (2010) 120:1063-1071
Klingler, J. et al. “Aphid resistance in Medicago truncatula involves antixenosis and phloem-specific, inducible antibiosis, and maps to a single locus flanked by NBS-LRR resistance gene analogs,” Plant Physiol. 137:1445-1455 (2005)
Lawton et al., Plant Mol. Biol. 9:315 324, 1987
Li Y. et al., “Soybean aphid resistance genes in the soybean cultivars Dowling and Jackson map to linkage group M,” Molecular Breeding 19:25-34 (2007)
Li Y. et al., “Soybean defense responses to the soybean aphid,” New Phytologist 179(1):185-195 (2008)
Mensah, C., et al., “Resistance to soybean aphid in early maturing soybean germplasm,” Crop Sci. 45:2228-2233 (2005)
Milligan, S. B. et al. “The root knot nematode resistance gene Mi from tomato is a member of the leucine zipper, nucleotide binding, leucine-rich-repeat family of plant genes,” Plant Cell 10:1307-1320 (1998)
Nickell et al., “Registration of ‘Dwight’ Soybean,” Crop Sci. 38:1398 (1998)
Nickell, C. D. of al. “Registration of ‘Loda’ soybean, Crop Sci. 41:589-590 (2001)
Odell et al., “Identification of DNA sequences required for activity of the cauliflower mosaic virus 35S promoter Nature 313:810 812 (1985)
Patterson and Ragsdale, “Assessing and managing risk from soybean aphids in the North Central States,” Crop Sci. 44:98-106 (2004)
Rossi, M. et al., “The nematode resistance gene Mi of tomato confers resistance against the potato aphid,” Proc. Nat'l Acad. Sci. USA 95:9750-9754 (1998)
Schmutz, J., et al. (Jan. 14, 2010), “Genome sequence of the paleopolyploid soybean,” Nature 463: 178-183
Soybean Linkage Map available on the USDA Soybean Linkage website with an Internet address following “http:// of bfql.anri.barc.usda.gov/cqi-bin/soybean/Linkage.pl (2006)
Sun, Z. et al., “Study on the uses of aphid-resistance character in wild soybean, I. Aphid-resistance performance of F2 generations from crosses between cultivated and wild soybean; Soybean Genetics News letter 17:43-48 (1990)
Walker et al., Proc. Natl. Acad. Sci. (U.S.A.) 84:6624 6628, 1987
Williams 82 chromosome 7 draft genome sequence at the worldwide web address: phytozome.net/soybean (2008)
Yang et al., Proc. Natl. Acad. Sci. (U.S.A.) 87:4144 4148, 1990)

1. An isolated, synthetic or recombinant nucleic acid molecule comprising a sequence having at least or about 95% sequence identity to a sequence selected from the group consisting of:
(a) SEQ ID NO:17;
(b) nucleic acid sequences encoding a polypeptide having an amino acid sequence of any of SEQ ID NOS:1-16 and combinations of said amino acid sequences;
wherein said nucleic acid molecule is capable of conferring, or participating in conferring Aphis glycines resistance to a soybean plant.

2-3. (canceled)

4. An isolated polypeptide having at least or about or 95% sequence identity or similarity to a polypeptide encoded by a nucleic acid molecule of claim 1, wherein the polypeptide is capable of conferring or participating in conferring Aphis glycines resistance on a soybean plant.

5. The isolated polypeptide of claim 4 having at least or about 95% sequence identity or similarity to a polypeptide having a sequence selected from the group of amino acid SEQ ID NOS:5-15.

6. The isolated polypeptide of claim 5 having at least or about 95% sequence identity or similarity to a polypeptide having a sequence of amino acid SEQ ID NO:7.

7. The isolated polypeptide of claim 5 having at least or about 95% sequence identity or similarity to a polypeptide having a sequence of amino acid SEQ ID NO:13.

8. An isolated, synthetic or recombinant nucleic acid molecule encoding a polypeptide of claim 5.

9. An isolated, synthetic or recombinant nucleic acid molecule of claim 1 encoding a polypeptide having a sequence of amino acid SEQ ID NO:7 or SEQ ID NO:13.

10. The nucleic acid molecule of claim 1 comprising the sequence of SEQ ID NO:17.

11. A nucleic acid molecule of claim 1 wherein said nucleic acid sequence encoding a polypeptide is selected from the group of sequences encoding polypeptides having sequences of SEQ ID NOS: 5-15

12-16. (canceled)

17. A method of generating a nucleic acid molecule capable of conferring Aphis glycines resistance on a soybean plant comprising: obtaining the nucleic acid molecule of claim 1 and modifying one or more nucleotides in said nucleic acid to another nucleotide, deleting one or more nucleotides in said nucleic acid molecule, or adding one or more nucleotides to said nucleic acid molecule to obtain a modified nucleic acid molecule, wherein the modified nucleic acid molecule encodes an polypeptide capable of conferring Aphis glycines resistance on a soybean plant.

18-20. (canceled)

21. A nucleic acid probe for identifying a nucleic acid encoding a polypeptide conferring or capable of conferring Aphis glycines resistance on a plant wherein the probe comprises at least about 10 or at least about 75 consecutive bases of the nucleic acid molecule of claim 1, wherein the probe is capable of identifying the nucleic acid by hybridization under highly stringent conditions.

22-24. (canceled)

25. A method for identifying or isolating a nucleic acid sequence capable of conferring or contributing to conferring Aphis glycines resistance to a soybean plant, said method comprising hybridizing a probe comprising a nucleic acid molecule of claim 1, a fragment thereof having at least about 12 or at least about 70 base pairs, or a nucleic acid molecule having a fully complementary sequence to said nucleic acid molecules, to germplasm of a soybean under highly stringent hybridization conditions.

26-33. (canceled)

34. An array comprising an immobilized nucleic acid comprising the nucleic acid molecule of claim 1.

35. (canceled)

36. A gene capable of conferring or contributing to conferring Aphis glycines resistance to a plant transformed to contain said gene, wherein said gene comprises a nucleic acid molecule encoding one or more polypeptides having sequences selected from the group consisting of SEQ ID NOS:1-16 and combinations thereof.

37. A computer storage medium having recorded thereon a sequence selected from the group consisting of SEQ ID NOS:1-55, together with information identifying each of said sequences.

38. A method of selecting a plant or plant germplasm with resistance to Aphis glycines, from one or more plants or plant germplasms, said method comprising:
(a) detecting, by marker-assisted selection (MAS), in the plant(s) or germplasm(s) at least one allele in at least one marker locus that is associated with an Aphis glycines resistance locus flanked by SNP46169.7 or KIM5 and markers mapping within 5 cM thereof and KIM3 and markers mapping within 5 cM thereof; and
(b) selecting the plant(s) or germplasm(s) comprising the at least one allele in said at least one marker locus, thereby selecting a soybean plant having Aphis glycines resistance.

39. (canceled)

40. The method of claim 38 wherein the marker locus is selected from the group consisting of marker loci identified by markers ss107918249, 27A SNP456169.7, KIM5, SNP65906.2, 56B, KIM3, 21A, 25A, 83A, SNP7623, ss107913360, SNP442-1688, and SNP86377 and markers mapping within 5 cM thereof.

41-45. (canceled)

46. A plant selected by the method of claim 38.

47-49. (canceled)

50. A method for producing one or more primers for markers associated with Rag1 Aphis glycines resistance, said method comprising:
(a) providing a first nucleotide sequence of soybean chromosome 7 from soybean plant having Rag1 resistance to Aphis glycines wherein said first nucleotide sequence maps to a DNA interval between marker SNP46169.7 and KIM3 or between markers within 5 cM of either or both of said markers;
(b) providing a second nucleotide, sequence corresponding to said first sequence from a plant known to lack Rag1 Aphis glycines resistance;
(c) selecting at least one forward and reverse marker primer pair with oligonucleotide lengths between 15 and 75 base pairs from said first nucleotide sequence;
(d) separately amplifying genomic DNA from said primers in media containing said susceptible and said resistant plants, respectively, to form amplification products;
(e) selecting amplification products which are the only amplification products produced by said primers in each medium;
(f) determining the presence of polymorphisms between the selected amplification products from the susceptible and resistant soybean DNA; and
(g) selecting primers that produce polymorphic amplification products as primers for markers associated with Rag1 Aphis glycines resistance.

51-56. (canceled)

57. A nucleic acid probe comprising a sequence between about or 10 and about or 75 base pairs having a nucleotide sequence comprised in a nucleic acid molecule of claim 1 and comprising at least one polymorphism compared to a corresponding sequence from a soybean variety not having Rag1 resistance.
